(19) United States
(12) Patent Application Publication
(10) Pub. No.: US 2024/0026383 A1
(43) Pub. Date: Jan. 25, 2024

(54) UNIVERSAL RIBOSWITCH FOR INDUCIBLE GENE EXPRESSION

(71) Applicant: WAGENINGEN UNIVERSITEIT, Wageningen (NL)

(72) Inventors: Constantinos PATINIOS, Renkum (NL); Sjoerd Constantijn Arnoud CREUTZBURG, Bennekom (NL); John VAN DER OOST, Renkum (NL); Raymond Hubert Josephe STAALS, Ede (NL)

(21) Appl. No.: 18/248,080

(22) PCT Filed: Oct. 7, 2021
(86) PCT No.: PCT/EP2021/077682

§ 371 Date: Apr. 6, 2023

(30) Foreign Application Priority Data
Oct. 8, 2020 (GB) 2015944.8

(51) Int. Cl. C12N 15/90; C12N 15/11; C12N 9/22; C12N 15/67; C12N 15/70; C12N 15/78; C12N 1/20

(52) U.S. Cl. CPC C12N 15/902; C12N 15/11; C12N 9/22; C12N 15/67; C12N 15/70; C12N 15/78; C12N 1/20; C12N 2310/20; C12N 2800/80; C12N 2800/101; C12N 2840/002

(57) ABSTRACT

Aspects described herein relate to methods for controlling expression of RNA and polypeptides of interest using a tuneable self-splicing intron. Specifically, there is provided modified 5′ and 3′ exons of the T4 td intron which function as a tuneable self-splicing intron that can be introduced to any gene of interest to multiple spots in the open reading frame therefore allowing the intron to be inserted without changing the amino acid sequence of the protein of interest. Methods and a system for inducer controlled modification of a target genomic locus in a cell are also provided herein. The invention further provides kits for expressing an RNA of interest or a polypeptide of interest, and wherein the expression is in transformed host cells under the control of an inducer molecule.

This invention relates to the field of biotechnology and to the control of gene expression in organisms; more particularly the control of gene expression in genetically modified cells of organisms, wherein the genetic modification allows for expression of a protein or polypeptide of interest (POI) by the cell in reaction to a control mechanism, usually involving a switch responsive to an inducer molecule. The invention also relates to the field of CRISPR-Cas gene modification and the control of protein expression in artificial CRISPR-Cas systems.

### BACKGROUND

In biotechnology, control of gene expression is a desired attribute. Such control may provide several advantages such as avoiding toxicity, avoiding by-product formation and tuning of metabolic pathways. Currently, several methods have been developed for controlling gene expression e.g. different strength promoters (strong, weak, inducible promoters), different strength ribosomal binding sites (RBSs) and Riboswitches (amongst others).

Riboswitches are used for controlling gene expression, more specifically protein translation, and they have been used widely in the area of biotechnology (Breaker 2011 Breaker, R. R. (2011) “Prospects for riboswitch discovery and analysis” Molecular Cell 43 (6): 867-879.). Riboswitches see a wide application in biotechnology due to their simple design and their inducibility through an inducer molecule (e.g. theophylline).

Whilst riboswitches are a great technology to regulate gene expression, (depending on their structure) they can be leaky, they require knowledge of the 5′ untranslated region (UTR) sequence of the gene of interest and they may be complex to engineer to a non-model organism. For this reason, a more universal, easily applicable riboswitch is needed that will simplify the engineering process and will ensure control of gene expression/translation amongst a variety of organisms and genes.

A great example for the applicability of a universal gene expression/translation system is for controlling the Clustered Regularly Interspaced Short Palindromic Repeat-Cas (CRISPR-Cas). Homologous recombination (HR) combined with CRISPR-Cas counterselection is a powerful approach to perform genome editing with high editing efficiencies. However, to achieve high editing efficiencies, HR should precede CRISPR-Cas counterselection. CRISPR-Cas tools will, therefore, be inefficient unless the CRISPR-Cas module is tightly regulated. Several regulation systems have been developed to control the expression and activity of the CRISPR-Cas module (Davis, K. M., et al., (2015) “Small molecule-triggered Cas9 protein with improved genome-editing specificity” Nature Chemical Biology 11 (5): 316-318; Zetsche et al., (2015); Nihongaki et al. (2015) “Photoactivatable CRISPR-Cas9 for optogenetic genome editing” Nature Biotechnology 33 (7): 755-760; Liu et al. 2016 “A chemical-inducible CRISPR-Cas9 system for rapid control of genome editing” Nature Chemical Biology 12 (11): 980; Cañadas et al. (2019) RiboCas: a universal CRISPR-based editing tool for Clostridium” ACS Synthetic Biology 8 (6): 1379-1390; Tang et al. (2017) “Aptazyme-embedded guide RNAs enable ligand-responsive genome editing and transcriptional activation” Nature Communications 8 (1): 1-8. Siu and Chen (2019) “Riboregulated toehold-gated gRNA for programmable CRISPR-Cas9 function” Nature Chemical Biology 15 (3): 217-220; Kundert et al. (2019) “Controlling CRISPR-Cas9 with ligand-activated and ligand-deactivated sgRNAs” Nature Communications 10 (1): 1-11; Moroz-Omori et al. (2020) “Photoswitchable gRNAs for Spatiotemporally Controlled CRISPR-Cas-Based Genomic Regulation” ACS Central Science. Whilst the existing approaches are suitable for the organism, Cas protein or gRNA of interest, these solutions are typically not universally applicable.

For this reason, a more universal, easily applicable riboswitch is needed that will simplify the engineering process and will ensure control of gene expression amongst a variety of organisms and genes.

Thompson, K. M., et al., (2002) “Group I aptazymes as genetic regulatory switches” BMC Biotechnology 2 (1): 21 describes the attachment of a theophylline aptamer to the group I self-splicing T4 td intron to control and induce the thymidylate synthase gene in E. coli. The Wild Type (WT)/parental td intron in the thymidylate synthase gene was substituted with a number of different theophylline-dependent self-splicing introns. Importantly, the insertion position of the theophylline-controlled self-splicing intron was exactly the same as the parent td intron and so no modification was made to any of the 5′ or 3′ exons of the intron-gene complex. Nonetheless, the P6a stem loop was modified to be theophylline responsive and so Thompson K. M., (et al.) created an inducible version of the self-splicing td intron.

A problem with the inducer-controlled riboswitch described by Thompson et al. (2002) is that it cannot be transferred to other genes (other than the td gene), because this causes disruption of the amino acid sequence of the expressed protein. Therefore, the Thompson et al. (2002) riboswitch is not universal and it is restricted to the td gene.

Recently, a Cas system controlled by ligand-responsive riboswitch or known as RiboCas was reported as a universal genome editing tool in Clostridium species by Cañadas et al., (2019) “RiboCas: a universal CRISPR-based editing tool for Clostridium” ACS Synthetic Biology 8 (6): 1379-1390. RiboCas works by placing a riboswitch within the 5′ untranslated region of mRNA (5′ UTR). The riboswitch creates a loop which prevents the ribosome from binding at the Shine-Dalgarno (SD) sequence at the ribosomal binding sites (RBS), thereby inhibiting translation initiation. In the presence of a ligand (theophylline), the loop shifts and releases the SD sequence allowing the translation initiation to occur. This strategy of controlling the gene expression is a useful alternative when an inducible promoter is not an option.

The interaction between SD and anti-SD (the complementary sequence on the 16S rRNA of the ribosome) has been assumed to be a conserved and universal mechanism of translation initiation in prokaryotes (see Schmeing and Ramakrishnan (2009) “What recent ribosome structures have revealed about the mechanism of translation” Nature 461 (7268): 1234-1242). However, Nakagawa et al. (2010) “Dynamic evolution of translation initiation mechanisms in prokaryotes” Proceedings of the National Academy of Sciences 107 (14): 6382-6387 is a more recent comparative analysis of several prokaryotes and indicates that SD-independent translation is much more widespread than previously estimated. Alternative mechanisms are also reported in Nakagawa et al. (2010), which use ribosomal protein 51 (RPS1) or leaderless mRNAs that lack their 5′ UTR.

Chen, S. et al. (2007) “Characterization of strong promoters from an environmental Flavobacterium hibernum strain by using a green fluorescent protein-based reporter system” Appl. Environ. Microbiol. 73 (4): 1089-1100 describe how in some AT-rich prokaryotes, the common prokaryotic SD (GGAGG) (SEQ ID NO: 1) appears not to be conserved in the 5′UTR, although the anti-SD sequence (CCUCC) (SEQ ID NO: 2) is present at the 3′ end of the 16S rRNA. Accetto, T., & G. AvgAtin. (2011) “Inability of Prevotella bryantii to form a functional Shine-Dalgarno interaction reflects unique evolution of ribosome binding sites in Bacteroidetes” PloS one 6 (8) describe how low GC content at the 5′UTR indicates a reduced tendency to form secondary structures, implying that utilizing riboswitch in the 5′UTR region may result in a leaky system.

All of the aforementioned limitations make the recently described RiboCas tool difficult to apply in species with SD-independent translation systems.

CRISPR-Cas has become a regular genome engineering tool for many prokaryotes and eukaryotes. Application of this technology ranges from gene editing to controlling the expression of a gene of interest (GOI) through silencing or induction. Typical CRISPR-Cas applications include:

CRISPR-Cas-mediated genome editing involving non-homologous end joining (NHEJ) and the homology-directed repair (HDR) systems that repair the double strand breaks (DSBs) generated by active Cas proteins. In case of a DSB in a gene, NHEJ creates insertions or deletions (indels) that often disrupt the function of the gene. HDR can be used for any type of precision editing, for example, substituting a base pair (gene therapy), removing a gene (knock-out) or introducing a gene (knock-in).

CRISPR base editing involving fusion of a dead Cas (dCas) or a nickase Cas (nCas) to a base editor. A cytidine deaminase (e.g. APOBEC1) or an adenine deaminase (TadA) have been used so far for base editing (Eid et al., 2018) “CRISPR base editors: genome editing without double-stranded breaks” Biochemical Journal 475(11): 1955-1964). Such a tool is used to create single base edits in the target of interest and thereby fix or destroy the GOI. This tool can be considered as CRISPR-Cas-mediated genome editing as well, but an important difference with the aforementioned editing approaches is that it does not generate DSBs.

CRISPR prime editing involving a fusion of a dead Cas (dCas) to an engineered reverse transcriptase. A prime editing guide RNA (pegRNA) is sequence specific for the target site and encodes the desired edit.

CRISPR transposition involving a catalytically inactive Cas protein (dCas) linked to a transposase, that results in guide-dependent integration of DNA fragments.

CRISPR interference involves a catalytically inactive Cas protein (dCas) which mediates the downregulation of gene expression by binding to the promoter or the coding sequence of the GOI. This can be considered as gene silencing.

CRISPR activation involving the fusion of a dCas protein to a transcription factor or an induction element that mediates the recruitment of the RNA Polymerase (RNAP) and thereby activates the expression of the GOI.

The problem is that whilst many state of the art technologies have been developed, and two main Cas proteins (Cas9 and Cas12a) have been widely used for genome engineering, strict control of the expression of the Cas proteins is limited in various ways.

Inducible promoters may be used whereby expression of the Cas protein is usually under the control of the inducible promoter e.g. a tetracycline promoter, which blocks the expression of the Cas protein when the inducer (tetracycline in this case) is absent. Addition of the inducer, allows for the expression of the protein which mediates gene editing, interference, activation, etc. A strict level of control of the Cas protein is especially important in HDR applications in prokaryotes that often lack NHEJ and often have a poorly active HDR system. Cas nuclease is used for counter-selection (i.e. to eliminate the wild type and enrich the desired recombinant) which implies that HDR must precede the nuclease activity of the Cas protein. In other words, HDR should take place before the Cas protein is able to target the genome and bring about cell death. Strict regulation of Cas protein expression by an inducible promoter is often the best option to delay the activation of Cas protein as a counter-selection tool and to provide enough time for homologous recombination to occur (see Mougiakos et al., (2017) “Efficient genome editing of a facultative thermophile using mesophilic spCas9” ACS Synthetic Biology 6.5: 849-861; and Cañadas et al., (2019) Supra).

Despite the existence of inducible promoters useful in model microorganisms such as E. coli and S. cerevisiae (e.g. Lactose/IPTG, Arabinose, Rhamnose, Galactose, Maltose, Xylose) or useful in human cells (e.g. TetR), the “leakiness” of such promoters may hinder the efficiency of HDR CRISPR-Cas. This problem becomes more apparent in non-model organisms like Flavobacterium species or Clostridia species, for which strictly inducible promoters are not really known. For example, the only reported inducible promoter that works in Flavobacterium requires a low temperature (12° C.) to be active (see Gómez et al., 2015) “Development of a markerless deletion system for the fish-pathogenic bacterium Flavobacterium psychrophilum” PLoS One 10.2: e0117969, and this low temperature sacrifices activity of the Cas nuclease. Such absence of suitable inducible promoters for controlling expression of Cas proteins, together with very low HR efficiencies means that there is little motivation to attempt to try and engineer a non-model organism using a CRISPR-Cas system.

Several microorganisms (e.g. Bacillus smithii) can grow at elevated temperatures e.g. 55° C. and above. At such high temperatures the basic cellular functions, including homologous recombination, are active whereas the Cas protein (spCas9 in this case) is inactive (see Mougiakos et al. (2017) Supra). The ability of the microorganism to grow and replicate at high temperatures allows for sufficient time for homologous recombination to occur before shifting to a temperature (37° C.) where the Cas protein is active and acts for counter-selection. Whilst this approach is successful for Bacillus smithii, it is a thermophile-specific method and so cannot be applied to other organisms.

CRISPR-Cas base editing, although very promising, suffers many off-target effects on DNA and RNA (see Zuo et al., (2019) “Cytosine base editor generates substantial off-target single-nucleotide variants in mouse embryos” Science 364.6437: 289-292; Xin et al., (2019) “Off-Targeting of Base Editors: BE3 but not ABE induces substantial off-target single nucleotide variants” Signal transduction and targeted therapy 4.1: 1-2. Zhou et al., (2019) “Off-target RNA mutation induced by DNA base editing and its elimination by mutagenesis” Nature 571.7764: 275-278). This off target problem has been attributed to the high protein levels (Base Editor-Cas fusion) followed by the lack of specificity of the base editor.

Pichler A. & Schroeder R. (2002) “Folding Problems of the 5′ Splice Site Containing the P1 Stem of the Group I Thymidylate Synthase Intron” J. Biol. Chem 277 (20) 17987-17993 is a scientific publication describing an in vitro cleaving assay for the thymidylate synthase (td) group I intron. Pichler et al., checked the effect of the 5′ splice site and showed that it can tolerate substitutions. However, they did not use this knowledge to characterize further the substitutions and use them to control the expression of any gene of interest. Pichler et al. describe a limited assessment of the effect of alterations at the P1 and P2 stem loop (5′ exon of the intron) and how they affect the self-splicing activity of the T4 td intron. Pichler et al. found that alterations at the −4 to −6 positions can alter the splicing activity of the intron in vitro. Pichler et al. describe how both a stable variant (−4P, −5P, −6P) and a destabilized variant (−4M, −5M and −6M) have better self-splicing activity when compared to the unmodified (WT) intron. Pichler et al. does not describe the effect of modifying the −7 or +296 bases on self-splicing. Whilst Pichler et al. describe particular alterations at the 5′ exon of the T4 td intron, they do not describe or suggest the use of using any modified T4 td intron for the purpose of controlling gene expression.

WO2016/166310 (Wageningen Universiteit) discloses an intronic, self-splicing riboswitch configured for enzyme-product specificity by introducing an appropriate aptamer. This then provides a sensing-expression construct, whereby the presence of an enzyme product in the cell triggers self-splicing of the intron sequence to restore the reading frame of the reporter gene and as such to drive expression of the gene product. The sensing construct expresses a protein which marks the cell or permits its growth or survival in or on an otherwise selective media. In this way, introduction or the presence of such product sensing-reporter constructs in cells can be harnessed to provide a multi-parallel rapid screening of cells or libraries for desirable enzyme variants. Modifications of the Td intron strictly followed those made by Pichler et al. (see above). Also described is using at least 2 self-splicing introns to minimise “leakiness” of the expression control. In particular, the self-splicing introns were introduced into a T7 polymerase for downstream control of a GFP gene serving as the GOI.

WO2018/083128 (Wageningen Universiteit) discloses how in the absence of efficient non-homologous end joining (NHEJ) repair mechanisms in the majority of microbes, double stranded DNA break (DSDB) typically leads to cell death. Therefore methods of microbial gene editing are provided involving plasmid transformation. Both homologous recombination and Cas9 site-specific gene editing events can be used together. Single or multiple plasmid approaches are used. In a method of counter-selection of microbes for a desired genetic change, a two-phase approach is used whereby a switch is made from a higher growth temperature phase favouring homologous recombination (HR)—as opposed to a Cas9 site-directed nuclease activity- to a lower growth temperature phase at which the Cas9 site directed nuclease activity takes place. This has the effect whereby the Cas9 site-directed nuclease activity has counter selecting activity, removing microbes which do not have a desired modification introduced beforehand by HR. The population of microbes surviving after the temperature switch counter selection is thereby enhanced for the desired modification.

### BRIEF SUMMARY OF THE DISCLOSURE

The inventors found that the splicing activities of the modified T4 td intron seen by Pichler et al. in vitro do not correspond to what is found to happen in vivo. By assessing a range of modifications in an in vivo system the inventors have surprisingly found that it is possible to modify the exon sequence portions of the T4 td self-splicing intron and achieve differential splicing activities. Therefore the inventors have discovered how to “tune” a self-splicing intron to work when inserted into a gene in an in vivo td expression system. The modified introns were placed either into the ORF of a gene of interest, just after the start codon of a gene of interest, or just before a start codon of a gene of interest.

The inventors have therefore discovered and developed a universal riboswitch that can be applied to virtually any gene and organism of interest. This universal riboswitch can be applied to prokaryotes and eukaryotes for the induction of protein expression, including RNA transcription. More particularly the invention has utility in relation to CRISPR-Cas engineering, including in organisms which have so far proved intractable for genetic modification.

More specifically, the inventors modified the 5′ and 3′ exons of the T4 td intron which function as a tuneable self-splicing intron that can be introduced to any GOI to multiple spots in the ORF, e.g. allowing the intron to be inserted without changing the amino acid sequence of the protein of interest. The inventors have also introduced Tag sequences which offer an additional advantage of simpler genetic manipulation work when constructing particular desired genetic sequences, since they can simply be added to the N-terminus of the POI. Each Tag sequence can have a different splicing activity and therefore a titration of inducible effect is achievable.

The combination of modification of the 5′ and 3′ exon sequence together with the provision of Tag sequences makes the inducible riboswitch tool universal for any GOI and for any organism of interest.

In accordance with the present invention there is provided a method for controlling expression of a polypeptide of interest (P01) in a cell, comprising:
(A) providing a cell comprising a polynucleotide construct, the polynucleotide construct comprising:
   i. a promoter functional in the cell;
   ii. a polynucleotide portion encoding said P01; and
   iii. a polynucleotide portion encoding at least one self-splicing intron which includes 5′ and 3′ exon nucleotide sequences, wherein the self-splicing activity of the intron is controlled by an inducer molecule;
      wherein the inducer-controlled self-splicing intron is located (a) at or 5′ of the start of the polynucleotide
      portion encoding the POI, or (b) within the polynucleotide portion encoding the P01;
(B) subjecting the cell to conditions which express polypeptides in the cell and thereby the transcription of the polynucleotide construct into RNA transcripts in the cell; and
(C) subjecting the cell to conditions which cause a concentration of inducer molecule to promote the self-splicing activity of the intron in the transcripts;
   thereby resulting in expression of the POI.

The invention further provides a method for controlling expression of an RNA of interest (ROI) in a cell, comprising:
(A) providing a cell comprising a polynucleotide construct, the polynucleotide construct comprising:
   i. a promoter functional in the cell;
   ii. a polynucleotide portion encoding the ROI; and
   iii. a polynucleotide portion encoding at least one self-splicing intron which includes 5′ and 3′ exon sequences, wherein the self-splicing activity of the intron is controlled by an inducer molecule;
      wherein the inducer-controlled self-splicing intron is located (a) at or 5′ of the start of the polynucleotide portion encoding the ROI, or (b) within the polynucleotide portion encoding the ROI;
(B). subjecting the cell to conditions which expresses the polynucleotide construct into RNA transcripts in the cell; and
(C) subjecting the cell to conditions which produces a concentration of inducer molecule which promotes the self-splicing activity of the intron in the RNA transcript to produce the ROI;
   thereby resulting in the expression of the ROI.

In accordance with the invention, the locating of an inducible self-splicing intron in a gene of interest (GOI), which is almost always a different gene from where the self-splicing intron is found in nature, means that the inducible self-splicing intron is located in a non-native, non-WT position in a gene. This surprising effect is tunable via certain modifications to the 5′ and 3′ exon regions of the self-splicing intron, allowing for universal applicability to prokaryotes and eukaryotes, and to permit addition of tag sequences which allow differential splicing activity in reaction to inducer. What is unexpected in this invention is that by changing the location of the intron from a naturally occurring position in a genetic sequence to a novel position in a different genetic sequence, that functionality of the self-splicing can be maintained. This is surprising because hitherto it was expected that intron functionality would be dependent a multiplicity of factors which have all to be in place, such as secondary structure of the mRNA, which is dependent on the exact sequence context (i.e. the primary sequence of the total RNA molecule). By introducing different mutations in the 5′ and/or 3′ exon sequences of the T4 td intron, the inventors have successfully managed to decrease or increase the splicing activity of the intron, thereby creating a library of tunable self-splicing intron variants. The tuned activity of the self-splicing intron is unexpected, especially since increased activity shown by some of the variants (see for example in FIG. 8, −4M, −5W, −6P or −4M, −5P, −6P).

What is also advantageous with the self-splicing introns of the invention is that the inducible system they provide is SD-independent and can be used for any GOI in any organism (without substantially interfering with the coding sequence). This also allows inducible expression of any ROI or POI in organisms where there are limited or no inducible promoters available.

The protein of interest (POI) may be any desired protein which is needing to be expressed in the cell. POI are typically polypeptide macromolecules comprising 20 or more contiguous amino acid residues and may include, but are not limited to enzymes, structural proteins, binding proteins and/or surface-active proteins. The methods of the present invention are useful in the production of desirable proteins in the agricultural, chemical, industrial and pharmaceutical fields. POI may include those of therapeutic value or industrial value. Examples of POI include enzyme, binding protein, antibody or chimeric antibody.

The POI may be a protein which is already endogenous to the cell, optionally wherein the POI is modified in amino acid sequence compared to the native endogenous protein of the cell. Often the POI is a heterologous protein which is not normally expressed by the unmodified WT cell.

Advantageously, the present invention is of broad applicability and host cells may be selected from any archaea, prokaryotic or eukaryotic cells. The invention is applicable to commonly used host cells, for example prokaryotic cells, fungal cells, plant cells and animal cells commonly used for recombinant heterologous protein expression. Equally the invention is applicable to less commonly used host cells, including prokaryotic cells, or cells of prokaryotes or eukaryotes which have not yet been subjected to genetic modification.

Self-splicing introns may be adapted as described herein via the 5′ and 3′ exon sequences to provide the optimum inducer controlled self-splicing for the ROI or POI at the selected site of insertion. The most suitable sites of insertion for a given ROI and POI may be chosen using an selection algorithm of the kind described in Example 4 and/or 11. Following this, the better performing 5′ and 3′ exon variant sequences of the self-splicing introns of the invention are readily identified in accordance with methods described herein. The self-splicing introns and methods of the invention are thereby of universal applicability to any desired ROI or POI. There is additionally the opportunity of altering selected nucleotide residues of an exon sequence encoding a ROI or POI. Therefore the particular choice of variants of the 5′ and 3′ exons of the self-splicing introns of the invention may be married together with a modification of nucleotides in the sequence which is receiving the self-splicing intron. Such modifications are silent in the sense that they do not alter the encoded amino acid of the ROI or POI. In this way further optimization and universal application of the self-splicing introns of the invention may be achieved. Polynucleotide constructs as described herein in accordance with any aspect of the invention are preferably in the form of an expression vector. Suitable expression vectors will vary according to the recipient host cell and suitably may incorporate regulatory elements which allow expression in the host cell of interest and preferably which facilitate high-levels of expression. Such regulatory sequences may be capable of influencing transcription or translation of a gene or gene product, for example in terms of initiation, accuracy, rate, stability, downstream processing and mobility.

Polynucleotides, usually expression constructs, in accordance with any aspect of the invention defined herein, may be in the form of plasmids used to transform a host cell. Methods of transformation are well known to persons of skill in the art and include but are not limited to; heat shock, electroporation, particle bombardment, chemical induction, microinjection and viral transformation.

In any of the methods of the invention for expressing POI, the self-splicing intron may be located 3′ of and in-frame with the start codon (i.e. not causing a frameshift in the polynucleotide portion encoding the POI upon splicing) and the expressed POI comprises an amino acid tag sequence encoded by a polynucleotide sequence which includes the 5′ and 3′ exon nucleotide sequences of the self-splicing intron rendered contiguous by self-splicing of the intron; preferably wherein the amino acid tag sequence is an N-terminal amino acid tag in the expressed POI. For example, in this way an N-terminal tag can be added to or fused to the POI, whereby methionine encoded by the in frame start codon is followed by a tag encoded by the 5′ and 3′ exon sequences rendered contiguous by the splicing, which is then followed by the (further) amino acids of the POI. The methionine can be directly followed by the amino acids encoded by the 5′ and 3′ exon sequences, i.e. the self-splicing intron is then directly adjacent to the start codon. Alternatively, or one or more amino acids can be included in between the start codon and the self-splicing intron to create a longer tag.

In alternative methods of the invention, the self-splicing intron may be located within the polynucleotide portion encoding the ROI or POI, e.g. 3′ of and in-frame with the start codon (i.e. not causing a frameshift in the polynucleotide portion encoding the POI upon splicing), and preferably the expressed ROI or POI does not comprise a tag added to the ROI or POI, i.e. preferably the intron is inserted such that no changes in the ROI or amino acid sequence of the POI are made, e.g. by making use of the herein described modifications to the 5′ and/or 3′ exon sequences. Possible insertion sites in any ROI or POI can thus be determined, e.g. using the herein described script.

In alternative methods of the invention, the self-splicing intron is 5′ of the polynucleotide portion which encodes the POI and therefore is at or 5′ of the start codon, such that the polynucleotide portion is not disrupted by the self-splicing activity of the intron.

When a self-splicing intron is located 5′ of the start codon of a polynucleotide coding a POI, then this may be directly adjacent to the start codon, or upstream of the start codon but downstream of the ribosome binding site. In other words, the self-splicing intron may be inserted anywhere into the stretch of contiguous nucleotides between the ribosome binding site and the start codon. In some embodiments, this can be at most about 12 nt upstream of the start codon; and in other embodiments at most about 11 nt, 10 nt, 9 nt, 8 nt, 7 nt, 6 nt, 5 nt, 4 nt, 3 nt, 2 nt or 1 nt upstream of the start codon.

A person of skill in the art will understand that in the case of an intron 3′ of a start codon (e.g. 3′ of the start codon), the 5′ and 3′ exon sequences of the self-splicing intron will need to be such that no frame shift is caused in the polynucleotide encoding the POI, e.g. by presenting complete codons, and as such are inserted in reading frame to the nucleotide sequence.

In aforementioned methods of the invention, the polynucleotide construct may further comprise a polynucleotide sequence encoding an additional amino acid sequence. When present, an additional amino acid sequence may be a functional moiety, e.g. a protein purification or detection tag, a cellular localization sequence, or a fluorescent moiety. In some embodiments, this additional amino acid sequence, e.g. functional moiety, can be included in or added to the N-terminal tag sequence, so as to fuse this to the POI upon splicing (and translation).

In the methods of the invention two or more self-splicing introns may be present. In such embodiments where two or more self-splicing introns are present, at least one of them may be comprised in the polynucleotide portion from which the POI is expressed; optionally directly adjacent and in-frame with the start codon (i.e. not causing a frameshift in the polynucleotide encoding the P01). Additionally or alternatively at least one of the two or more self-splicing introns is 5′ of the polynucleotide portion which encodes the POI, i.e. of the start codon of the polynucleotide encoding the POI.

In embodiments concerning POI, all of the self-splicing introns may be comprised in the polynucleotide portion which encodes the POI, such as 3′ of the start codon; optionally wherein at least one of said two or more self-splicing introns is directly adjacent to and in-frame with the start codon of the POI (i.e. not causing a frameshift).

In embodiments concerning ROIs which are mRNA, the aforementioned aspects relating to methods for controlling expression of POI apply; the mRNA ROI as will be appreciated by a person of skill in the art, being an intermediate step in the process of protein expression.

In other embodiments concerning ROIs which are other than mRNA, then an RNA of interest (ROI) herein may be any of transfer RNA (tRNA), ribosomal RNA (rRNA), long non coding RNA (lncRNA), micro RNA (miRNA), small nucleolar RNA (snoRNA), PIWI-interacting RNA (piRNA), circular RNA (circRNA), small interfering RNA (siRNA), antisense RNA (aRNA), CRISPR guide RNA (gRNA) or crRNA or single guide RNA (sgRNA), trans-activating CRISPR RNA (tracrRNA), double stranded RNA (dsRNA), short hairpin RNA (shRNA), trans-acting siRNA (tasiRNA), repeat associated siRNA (rasiRNA), enhancer RNA (eRNA).

In the aforementioned examples of ROI, the RNA is encoded by a transcription unit and so at least one self-splicing intron in accordance with the invention may be present either within the transcription unit, or upstream thereof. More than one self-splicing intron may be used, with at least one present within the transcription unit and at least one present in the polynucleotide portion between the promoter and the transcription unit.

In methods of the invention, the self-splicing intron preferably comprises an aptamer which has binding affinity for the inducer molecule. The aptamers may be DNA, cDNA, RNA, or preferably RNA. Suitable aptamers may be 20-30 nt in length; optionally they are 20 nt, 21 nt, 22 nt, 23 nt, 24 nt, 25 nt, 26 nt, 27 nt, 28 nt, 29 nt or 30 nt in length. New aptamers can readily be developed for a required inducer molecule by means known in the art, preferable by a selection procedure such as Systematic Evolution of Ligands by Exponential Enrichment (SELEX) of aptamer fragments. In this way the specificity of each self-splicing intron in the invention can be adjusted for the cell or inducer required to be worked in.

Inducers of aptamers useful in accordance with the invention may be selected from: flavin mononucleotide, thiamine pyrophosphate, s-adenosylmethionine, s-adenosylhomocysteine, adenosylcobalamin, cyclic diguanylate, adenine, guanine, glycine, lysine, theophylline, 3-methylxanthine, caffeine, 1-methylxanthine, 7-methylxanthine, 1,3-dimethyl uric acid, hypoxanthine, xanthine, theobromine tetracycline, neomycin or malachite green, 2′-Deoxyguanosine, Magnesium, glucosamine-6-phosphate, 7-aminomethyl-7-deazaguanine, 7-cyano-7-deazaguanine, Aquacobalamin, Molybdenum cofactor, Tungsten cofactor, Tetrahydrofolate, Prequeusine-1, c-di-adenosine monophosphate, Cyclic guanosine monophosphate-adenosine monophosphate. Preferably the inducer is theophylline.

In various embodiments of the invention, the 5′ exon nucleotide sequence and/or 3′exon nucleotide sequence of the self-splicing intron are preferably modified compared to the respective wild type exon nucleotide sequence(s) of the intron.

Ordinarily, the self-splicing introns employed in any aspect of the invention described herein are Group I introns, although it is possible for Group II or Group III self-splicing introns to be used.

In preferred aspects, the self-splicing intron is the T4 td self-splicing intron wherein the 5′ exon sequence is NNNNNNGGT (SEQ ID NO: 3) and the 3′ exon sequence is CTN (SEQ ID NO: 4), preferably wherein the 5′ exon sequence is TTBYBDGGT (SEQ ID NO: 5) and the 3′ exon sequence is CTH (SEQ ID NO: 6) (wherein B=G/T/C, Y=C/T, D=G/A/T and H=A/T/C) optionally wherein the 5′ exon sequence is selected from TCCTCAGGT (SEQ ID NO: 7), TCCTCGGGT (SEQ ID NO: 8), TCCTTGGGT (SEQ ID NO: 9), TCCTCTGGT (SEQ ID NO: 10) or TTCTTGGGT (SEQ ID NO: 11); and the 3′ exon sequence is CTA (SEQ ID NO: 12).

In some aspects, the native exon portions of self-splicing introns may be modified at any or all of positions of −9, −8, −7, −6, −5, −4 and +296. Additionally or alternatively any or all of positions −3, −2, −1, +294 and +295 are not modified and so are kept as wild type in order to maintain self-splicing ability of the intron. This will provide more flexibility in placing the intron within a coding sequence without changing the amino acid sequence of the POI (i.e. no tag is added).

In some aspects, the self-splicing intron is the T4 td self-splicing intron and the exon sequences are modified compared to the respective wild type exon nucleotide sequence(s) of the intron, but the present invention optionally does not include a self-splicing intron with the following 5′ exon sequence: 5′ caccuuaggu 3′ (SEQ ID NO: 13) and/or the following 3′ exon sequence: 5′ cuat 3′ (SEQ ID NO: 14). Additionally or alternatively the present invention optionally does not include a self-splicing intron with the following 5′ exon sequence: 5′ uucuugggu 3′ (SEQ ID NO: 15) and/or the following 3′ exon sequence: 5′ cuac 3′ (SEQ ID NO: 16).

The invention also may exclude the following 5′ exon sequence: 5′ CACCTTAGGT 3′ (SEQ ID NO: 17) and/or the following 3′ exon sequence: 5′ CTAT 3′ (SEQ ID NO: 18). Additionally or alternatively the present invention optionally does not include a self-splicing intron with the following 5′ exon sequence: 5′ TTTCTTGGGT 3′ (SEQ ID NO: 19) and/or the following 3′ exon sequence: 5′ CTAC 3′ (SEQ ID NO: 20).

In other aspects, the T4 td self-splicing intron in the present invention does not include a 5′ exon sequence of CAAGGGT (SEQ ID NO: 21) or CTTGGGT (SEQ ID NO: 22) and/or a 3′ exon sequence of CTAC (SEQ ID NO: 20) or CTAA (SEQ ID NO 23).

In accordance with any aspect of the invention, a POI may be selected from any of:
i. a sequence specific DNA/RNA binding protein; preferably a meganuclease (MGN), zinc finger nuclease (ZFN), a TALEN, an RNA-guided nuclease or a DNA-guided nuclease;
ii. an RNA-guided nuclease; preferably a Crispr-Cas protein;
iii. a sequence-specific DNA binding protein lacking nuclease activity or a nickase; optionally fused to a heterologous functional moiety; preferably wherein the POI is a base editor or a prime editor.

In circumstances wherein the POI is ii) or iii), then polynucleotide preferably also further comprises a portion encoding a targeting RNA molecule, e.g. a guide RNA (gRNA) which directs ii) or iii) to a target locus in a DNA sequence. The CRISPR-Cas nucleases may be selected from any Cas Type I, Type II or Type III. More particularly, the Cas may be selected from Cas9, Cas12a (previously known as Cpf1) or Cas13 (previously known as C2c2); also any of Caw, Cas12b, Cas12c, Cas13a,b,c,d, Cas4, Csn2, Csf1, Csx10, Csx11, Cmr5, Csm2, Cas10, Csy1,2,3, Cse1,2, Cas10d, Cas8a,b,c, Cas5 or Cas3. The CRISPR-Cas nucleases may any variant from any species, whether well-known, e.g. from Streptococcus pyogenes (SpyCas9), or less commonly used such as from Geobacillus thermodenitrificans T12 (ThermoCas9) or Geobacillus stearothermophilus (GeoCas9).

In any of the methods described herein, separately of the inducer controlled self-splicing introns described herein, polynucleotides and expression vectors in host cells may be under primary inducer control using well-known induction systems for cell expression. As such, the polynucleotide constructs and expression vectors will comprise the necessary elements and an inducer molecule can be provided to the cell. When an inducer is provided exogenously to an host cell, then it may be a chemical compound (e.g. Dimethyl sulfoxide (DMSO), Doxycycline, Muristerone A; Ponasterone A). Suitable inducers also include, but are not limited to; Rhamnose, Arabinose or Isopropyl β-D-1-thiogalactopyranoside (IPTG).

Alternatively, expression of heterologous polynucleotides and expression vectors may be under the control of environmental factors such as heat shock, high or low pH; physiological factors such as glucose elevation or hypoxia; or developmental cues, such as cell density or growth phase. This permits a generic level of switching that can be used to turn cells on and off as regards heterologous gene expression.

The invention therefore includes an isolated polynucleotide, also to be termed a polynucleotide construct, comprising:
i. a promoter functional in a cell;
ii. a polynucleotide portion encoding an RNA of interest (ROI) or a polypeptide of interest (POI); and
iii. a polynucleotide portion encoding at least one self-splicing intron which includes 5′ and 3′ exon nucleotide sequences, wherein the self-splicing activity of the intron is controlled by an inducer molecule;
wherein the inducer-controlled self-splicing intron is located (a) at or 5′ of the start of the polynucleotide portion encoding the ROI or POI, or (b) within the polynucleotide portion encoding the POI or ROI.

Using such polynucleotides of the invention, the ROI may be translatable into the POI when the ROI is an mRNA.

1. In preferred aspects, the self-splicing intron is 3′ of an in-frame start codon (e.g. 3′ of the start codon) and a POI when expressed from the polynucleotide comprises an amino acid tag sequence encoded by a polynucleotide sequence which includes the 5′ and 3′ exon nucleotide sequences of the self-splicing intron rendered contiguous by self-splicing of the intron; preferably wherein the amino acid tag sequence is an N-terminal amino acid tag in or fused to the expressed POI, such as described in relation to the method aspects of the invention as hereinbefore described.

In other aspects, the self-splicing intron may be located within the polynucleotide portion encoding the ROI or POI, e.g. 3′ of and in-frame with the start codon (i.e. not causing a frameshift in the polynucleotide portion encoding the POI upon splicing), and preferably the expressed ROI or POI does not comprise a tag added to the ROI or POI, i.e. preferably the intron is inserted such that no changes in the ROI or amino acid sequence of the POI are made, e.g. by making use of the herein described modifications to the 5′ and/or 3′ exon sequences, such as described in relation to the method aspects of the invention as hereinbefore described.

In other aspects, the self-splicing intron is 5′ of the polynucleotide portion from which the ROI or POI is expressed and the said polynucleotide is not disrupted by the self-splicing activity of the intron; preferably wherein the self-splicing intron is 5′ of a start codon (e.g. 5′ of the start codon) of the polynucleotide encoding the POI.

The polynucleotide constructs of the invention described herein may further comprise a polynucleotide sequence encoding an additional amino acid sequence; optionally wherein the additional amino acid sequence is a functional moiety, e.g. a protein purification or detection tag, a cellular localization sequence, a fluorescent moiety. such as described in relation to the method aspects of the invention as hereinbefore described.

In some embodiments, two or more self-splicing introns may be used. Each of the self-splicing introns may be at a different location rather than contiguous or substantially contiguous. For example, a first self-splicing intron may be 5′ of the polynucleotide portion from which the ROI or POI is expressed, e.g. 5′ of a start codon (e.g. 5′ of the start codon), and a second self-splicing intron may be located at any position 3′ and in-frame with the start codon (i.e. not cause a frame shift in the polynucleotide encoding the P01); optionally directly adjacent to the start codon. There may be three, four or more self-splicing introns present and the positions may be selected independently.

Where there are two or more self-splicing introns, at least one is 3′ of the start codon; or at least one is 5′ of the start codon. The positioning of any self-splicing intron in polynucleotides of the invention may be as described in relation to the method aspects of the invention as hereinbefore described.

Where there are two or more self-splicing introns, each intron may be induced by the same inducer, or each intron may be induced by a respective different inducer molecules.

For self-splicing introns which are induced by an inducer molecule, these may preferably comprise an aptamer as the binding site for the inducer. The aptamer will have a binding affinity and degree of specificity for the inducer molecule; optionally wherein the inducer molecule is one selected from flavin mononucleotide, thiamine pyrophosphate, s-adenosylmethionine, s-adenosylhomocysteine, adenosylcobalamin, cyclic diguanylate, adenine, guanine, glycine, lysine, theophylline, 3-methylxanthine, caffeine, 1-methylxanthine, 7-methylxanthine, 1,3-dimethyl uric acid, hypoxanthine, xanthine, theobromine tetracycline, neomycin or malachite green, 2′-Deoxyguanosine, Magnesium, glucosamine-6-phosphate, 7-aminomethyl-7-deazaguanine, 7-cyano-7-deazaguanine, Aquacobalamin, Molybdenum cofactor, Tungsten cofactor, Tetrahydrofolate, Prequeusine-1, c-di-adenosine monophosphate, Cyclic guanosine monophosphate—adenosine monophosphate. A preferred inducer is theophylline.

In polynucleotides of the invention described herein, the 5′ exon nucleotide sequence and/or 3′exon nucleotide sequence of the self-splicing intron are preferably modified compared to the respective wild type exon nucleotide sequence(s) of the intron.

In a preferred embodiment of a polynucleotide of the invention, including for use in the methods of the invention, the self-splicing intron is the T4 td self-splicing intron. Modifications and variations of the 5′ and 3′ exons of this self-splicing intron are as described in connection with the aforementioned method aspects of the invention.

There are additional applications of the methods and polynucleotides of the invention with respect to the genetic modification of organisms and cells. Therefore in one aspect, the POI is selected from:
i. a sequence specific DNA/RNA binding protein; e.g. an argonaut; or preferably a meganuclease (MGN), zinc finger nuclease (ZFN), a TALEN, an RNA-guided nuclease or a DNA-guided nuclease;
ii. an RNA-guided nuclease; preferably a Crispr-Cas protein;
iii. a sequence-specific DNA binding protein lacking nuclease activity or a nickase; optionally fused to an heterologous functional moiety; preferably wherein the POI is a base editor or a prime editor.

Therefore, when the POI is ii) or iii), a polynucleotide of the invention as used in method applications of the invention may further comprise for convenience a portion encoding a targeting RNA molecule, e.g. guide RNA (gRNA) which directs the b) or c) to a target locus in a DNA sequence; optionally wherein the gRNA is under the control of a self-splicing intron. Alternatively, the targeting RNA molecule may be supplied directly or via expression from a separate expression construct introduced into the cell.

In an aspect of the invention, the POI may be an argonaut.

The invention includes expression vectors comprising any of the polynucleotides as herein described.

The invention also provides transformed cells for inducer molecule-controlled expression of an RNA of interest (ROI) or polypeptide of interest (POI) thereby, wherein such a cell comprises a polynucleotide as herein described, or an expression vector comprising such polynucleotides.

The invention further provides kits for expressing an RNA of interest (ROI) or a polypeptide of interest (POI) as hereinbefore described, and wherein the expression is in transformed host cells under the control of an inducer molecule. The kits can comprise:
i. a composition comprising a polynucleotide, an expression vector, or a transformed cell as hereinbefore described; and
ii. a composition comprising an inducer molecule which activates self-splicing activity of a self-splicing intron when expressed in a cell.

The invention includes a system for generating an RNA of interest (ROI) or a polypeptide of interest (POI), comprising a transformed cell as herein defined.

In other aspects, the invention provides a method of inducer controlled modification of a target genomic locus in a cell, comprising introducing or generating in the cell a ribonuclease complex comprising a Crispr-Cas nuclease and a gRNA molecule for the target genetic locus; wherein the Crispr-Cas nuclease and/or the gRNA is comprised as the ROI and/or POI in a polynucleotide construct or an expression vector as hereinbefore defined; and

2. subjecting the cell to a condition which causes a concentration of inducer molecule to promote the self-splicing activity of the intron, thereby resulting in expression of the Crispr-Cas nuclease and/or gRNA in the cell; optionally wherein an homologous repair (HR) template encoded by the same or different polynucleotide or expression vector, and the HR template is expressed in the cell.

The invention also includes a method of inducer-controlled base editing of a target genomic locus in a cell, comprising:
i. introducing or generating in the cell a ribonuclease complex comprising a base editor and a gRNA molecule for the target genetic locus, wherein the base editor and/or gRNA is comprised as the respective ROI or POI in a polynucleotide construct, polynucleotides or expression vector as hereinbefore defined; and
ii. (a) providing an inducer molecule to the cell, or (b) subjecting the cell to a condition which causes a concentration of inducer molecule to promote the self-splicing activity of the intron, thereby resulting in expression of the base editor and/or gRNA in the cell.

The invention further includes a method of inducer-controlled prime editing of a target genomic locus in a cell, comprising:
i. introducing or generating in the cell a ribonuclease complex comprising a prime editor and a prime editing guide RNA (pegRNA) molecule for the target genetic locus, wherein the prime editor and/or pegRNA is comprised as the respective ROI or POI in a polynucleotide construct or polynucleotides as hereinbefore defined; and
ii. (a) providing inducer molecule to the cell, or (b) subjecting the cell to a condition which causes a concentration of inducer molecule to promote the self-splicing activity of the intron, thereby resulting in expression of the prime editor and/or pegRNA in the cell.

In any of the aforementioned methods of the invention for genetic modification, an exogenous inducer molecule is preferably provided to the cell. Alternatively, the (a) the inducer molecule may be generated as a result of expression of a separate gene in the cell, wherein the separate gene is under the control of different expression regulatory elements; optionally wherein the different expression regulatory elements are responsive to a different inducer molecule and/or physical condition, e.g. temperature; or (b) the inducer molecule is naturally synthesized by the cell in response to chemical and/or physical condition to which the cell is subjected to. Such range of physical conditions have been referred to previously.

In any of the aforementioned methods of the invention for genetic modification of cells, a first polynucleotide may comprise a self-splicing intron under the control of a first inducer molecule, and a second polynucleotide comprises a self-splicing intron which is under the control of a second different inducer molecule. Similarly, further polynucleotides and inducer molecule combinations can be added. The inducer molecules may be same or different.

The invention herein includes a system for inducer controlled genetic modification of a cell, comprising at least a first expression vector, the first expression vector comprising a polynucleotide, a polynucleotide construct or expression vector as hereinbefore defined, wherein the respective POI or ROI is selected from:
i. a Crispr-Cas nuclease, and/or
ii. a gRNA, and/or
iii. an HR template.

The invention herein also includes a system for inducer controlled genetic modification of a cell, comprising at least a first expression vector, the first expression vector comprising a polynucleotide, polynucleotide construct or expression vector as hereinbefore defined,

wherein the respective POI or ROI is selected from:
i. a base editor, and/or
ii. a gRNA

Further, the invention includes a system for inducer controlled genetic modification of a cell, comprising at least a first expression vector, the first expression vector comprising a polynucleotide, polynucleotide construct or expression vector as hereinbefore defined, wherein the respective POI or ROI is selected from:
i. a prime editor, and/or
ii. a pegRNA

In any of the aforementioned systems of the invention, each individual POI and/ROI is preferably under the control of a respective self-splicing intron. More particularly, a first polynucleotide may comprise a self-splicing intron under the control of a first inducer molecule, and a second polynucleotide comprises a self-splicing intron which is under the control of a second different inducer molecule.

The ROI or POI from transcription/expression of the first polynucleotide in reaction to the respective first inducer molecule may provide the inducer molecule for a second, optionally third or more self-splicing introns encoded within second, third, etc. polynucleotides of the invention. The invention can therefore include modes of operation whereby an initial exogenously applied inducer molecule effect can be amplified by two or more inducer molecules within the cell which have been produced within the cell in reaction to the applied inducer molecule. Similarly, a physical induction can be used as the primary inducer resulting in production of a secondary ROI/POI which goes on to induce activity of a second self-splicing intron and production of a further ROI/POI. Clearly a person of skill in the art can design and operate a multiplicity of possibilities for cascade in control of expression of a desired, ultimate ROI/POI.

In the genetic modification method aspects, kits and systems of the invention defined herein is a SIBR-Cas tool which can often provide the only solution for Homology Directed Recombination (HDR) in prokaryotes. SIBR-Cas can be used virtually in any organism of interest for any of the previously demonstrated CRISPR-Cas applications and whenever it makes sense to control the expression of the Cas protein.

The invention therefore provides certain technical advantages of:
Highly controlled expression of a gene of interest (GOI) in order to generate a desired ROI or POI.
The self-splicing introns described herein are readily adopted in relation to any GOI, simply by adding the intron as Tag to the GOI. No further engineering is needed.
The self-splicing introns are universal in application and readily used in the cell of any organism of interest.
Because of the differential inducible activity of different tagged versions of the self-splicing introns (e.g., Tag1, Tag2, Tag3, Tag4, etc.) using a multiplicity of these allows for a “titratable” regulation of GOI/ROI/POI expression with an inducer.
As noted above, using more than one inducible self-splicing intron permits a tighter regulation over the GOI/ROI/POI.

Industrial applications of the invention described herein include:
Using the inducible self-splicing intron to control the expression of metabolic pathways. This can allow controlled expression of metabolic pathways which may allow higher product production and lower by-product formation.
Using the inducible self-splicing intron to control the expression of CRISPR-Cas proteins, thereby overcoming toxicity of these found in other expression systems, and improving Homologous Recombination.

### BRIEF DESCRIPTION OF THE DRAWINGS

Embodiments of the invention are further described hereinafter with reference to the accompanying drawings, in which:

FIG. 1 is a diagram of the predicted secondary and tertiary structure of the T4 td intron.

FIG. 2 is a simplistic cartoon version of the T4 td intron as shown in FIG. 1.

FIG. 3 shows the nucleotide sequences and structures of wild type P6a loop (left) and theophylline aptamer P6a loop (right) structures of the P6a loop of the T4 td intron

FIG. 4A shows greater detail of the 5′ and 3′ exonic sequences of the T4 td intron.

FIG. 4B shows the nucleotide positions of the 5′ and 3′ exons sequences of the T4 td intron which are modifiable in pursuit of the invention.

FIG. 5 is a schematic representation of the interruption of the LacZ gene with the T4 td intron. LacZ is interrupted with the T4 td intron.

FIG. 6 shows the results of LacZ activity for position −7 mutants. The asterisk indicates the WT intron.

FIG. 7 shows the results of LacZ activity of position +296 mutants. The asterisk indicates the wild-type intron.

FIG. 8 shows the results of LacZ activity of all possible combinations for pair (P), wobble pair (W) and mismatch (M) at positions −6 to −4. The wild type intron (*) is PWM/(UUG) and set to 1.

FIG. 9A is a cartoon of the insertion of the modified T4 td intron at the GOI.

FIG. 9B shows the insertion side at the FnCas12a gene.

FIG. 10 is a schematic representation of multiple introns inserted at different positions at the GOI.

FIG. 11 is a cartoon of a T4 td intron containing the theophylline aptamer at the P6 loop.

FIG. 12A shows the steps in DNA to RNA to Protein transcription of the intron-GOI constructs of the invention.

FIG. 12 B shows the steps in DNA to RNA to Protein transcription with induction of the theophylline dependent intron-GOI constructs of the invention

FIG. 12C compares the steps in DNA to RNA to protein transcription with the induction/translation of the tagged-intron-GOI constructs of the invention.

FIG. 12D shows theophylline dependent T4 td introns introduced as tags after the start codon “ATG” of the gene of interest (GOI).

FIG. 13 shows steps in DNA to RNA to protein transcription with the intron positioned directly before the ATG start codon of the gene of interest.

FIG. 14 shows full sequence information for (top) the self-splicing intron just after the ATG of the GOI, and (bottom) the self-splicing intron just before the ATG of the GOI.

FIG. 15 is a photograph of plates showing induced activity of FnCas12a in E. coli MG1655 transformed with the series of plasmids listed in Table 4.

FIG. 16. SIBR-Cas genome editing assays in E. coli MG1655. (A) Editing efficiency of the LacZ gene in E. coli MG1655. Blue/white screening was performed to distinguish the edited (white) from the unedited (blue) colonies when using either of the four different SIBR-Cas variants Int1 (0%), Int2 (1%±0.35%), Int3 (49%±8.75%) and Int4 (80%±5.57%) or the WT-FnCas12a (0%, n.d.). The percentage on top of each variant indicates the percentage of white colonies from the total number of colony forming units mL-1 (CFUs mL-1). (B) Representative plates of edited E. coli MG1655 cells at the LacZ locus using the four different SIBR-Cas variants (Int1-4; Int1 is the worst and Int4 is the best splicer). (C) Unbiased (omitting the presence of X-Gal in the medium) editing efficiency of the LacZ gene using the four different SIBR-Cas variants Int1 (0%), Int2 (0%), Int3 (29%±18.04%), Int4 (38%±6.41%) or the WT-FnCas12a (0%, n.d.). All of the NT controls showed 0% targeting efficiency. N.d.: not determined

FIG. 17 is a photograph of plates showing induced activity of FnCas12a in P. putida. P. putida was transformed with a series of plasmids listed in Table 6.

FIG. 18 shows the results of knock-out efficiency of the FlgM gene in P. putida using the Tagged-intron variants.

FIG. 19 shows the plasmid containing theophylline induced self-splicing intron and FnCas12a used to transform Flavobacterium IR1, the silent mutations introduced into the FnCas12a and the process of cleavage and ligation to produce a functional Cas12a.

FIG. 20 shows the experimental protocol for transformation and induction of Flavobacterium sp. IR1.

FIG. 21A is a schematic representation of SprF gene deletion in the genome of IR1.

FIG. 21B is a photograph of transformed Flavobacterium sp. IR1 cells grown on agar plates. Mutants are on the left side and WT are on the right side. FIG. 21C is a photograph of an Agarose gel electrophoresis of PCR of colonies (1-16) of transformed Flavobacterium sp. IR1 compared to WT grown for 72 hours. Also shown is a Sanger sequencing result of mutant and WT colonies.

FIG. 21D is a photograph of an Agarose gel electrophoresis of PCR of colonies (1-16) of transformed Flavobacterium sp. IR1 compared to WT grown for 96 hours. Also shown is a Sanger sequencing result of mutant and WT colonies.

FIG. 22A shows the transformation efficiencies for plasmids in Flavobacterium IR1.

FIG. 22B shows the theophylline toxicity for in Flavobacterium IR1y when in the growth medium.

FIG. 23 is an adopted diagram of the procedure for obtaining knock-outs in Flavobacterium IR1.

FIG. 24 shows the results for editing efficiencies of the SprF gene of Flavobacterium IR1 using the four different tagged-intron variants.

FIG. 25. Constructs used for testing the functionality of SIBR in the yeast Saccharomyces cerevisiae. pUDE731 constitutively expresses the WT FnCas12a. PL-319 splits the FnCas12a with a T4 td intron (which does not contain an aptamer) to a 5′ and 3′ exon. The intron is inserted before the RuvC I domain and the 5′ and 3′ flanking regions have been adopted to avoid amino acid change but still maintain splicing of the intron. PL-320 splits the FnCas12a with a SIBR T4 td intron (which contains a theophylline aptamer) to a 5′ and 3′ exon. The intron is inserted before the RuvC I domain and the 5′ and 3′ flanking regions have been adopted to avoid amino acid change but still maintain splicing of the intron.

FIG. 26. SIBR-Cas is functional in the yeast S. cerevisiae. pUD7E31, PL319 or PL320 were co-transformed either with a plasmid containing a non-targeting (NT) or a targeting (T) crRNA. Transformants were serially diluted (100, 10-1, 10-2) and plated on selective medium containing different concentrations of the theophylline inducer (0, 5, 10, mM).]

### DETAILED DESCRIPTION

Ribozymes and riboswitches are gene regulation systems found in a wide range of bacterial species. The catalytic and/or regulatory functionality of these RNA molecules relies on their primary, secondary and tertiary structures, making them great candidates for developing universal tools for regulating gene expression, without the use of proteins (Breaker, R. R. Riboswitches and the RNA world. Cold Spring Harbor perspectives in biology 4, a003566 (2012); Park, S. V. et al. Catalytic RNA, ribozyme, and its applications in synthetic biology. Biotechnology advances 37, 107452 (2019); Serganov, A. & Nudler, E. A decade of riboswitches. Cell 152, 17-24 (2013); Serganov, A. & Patel, D. J. Ribozymes, riboswitches and beyond: regulation of gene expression without proteins. Nature Reviews Genetics 8, 776-790 (2007); Weinberg, C. E., Weinberg, Z. & Hammann, C. Novel ribozymes: discovery, catalytic mechanisms, and the quest to understand biological function. Nucleic acids research 47, 9480-9494 (2019)) To this end, several studies used ribozymes and riboswitches to control the expression of a gene of interest (G01), but also for regulating the activity and function of CRISPR-Cas (Zhao, J., et al. Development of aptamer-based inhibitors for CRISPR/Cas system. Nucleic Acids Research (2020); Cañadas, I.s.C., et al. RiboCas: a universal CRISPR-based editing tool for Clostridium. ACS synthetic biology 8, 1379-1390 (2019); Tang, W., Hu, J. H. & Liu, D. R. Aptazyme-embedded guide RNAs enable ligand-responsive genome editing and transcriptional activation. Nature communications 8, 1-8 (2017); Siu, K.-H. & Chen, W. Riboregulated toehold-gated gRNA for programmable CRISPR-Cas9 function. Nature chemical biology 15, 217-220 (2019). Kundert, K. et al. Controlling CRISPR-Cas9 with ligand-activated and ligand-deactivated sgRNAs. Nature communications 10, 1-11 (2019); Park, S. V. et al. Catalytic RNA, ribozyme, and its applications in synthetic biology. Biotechnology advances 37, 107452 (2019)). Although quite successful, these approaches leave room for improvement. For example, the technology developed by Tang et al. (2017) requires base pairing of the CRISPR spacer sequence with the 5′ end of the hammerhead ribozyme; something that requires modification in case the CRISPR spacer needs to be changed. Moreover, the studies by Kundert et al. (2019), Siu et al. (2019) and Zhao et al. (2020) rely on the secondary structure of the Cas9 single guide RNA (sgRNA), which rules out the use of other CRISPR-Cas systems. Lastly, the RiboCas technology developed by Cañadas et al. (2019), regulates the expression of Cas9 by masking the RBS with a theophylline-dependent riboswitch. Whereas this technology is a smart alternative to previous approaches, it can be cumbersome to use either in organisms that do not use the canonical RBS sequence, or in cases that the secondary structure of the 5′ UTR sequence interferes with the theophylline aptamer (Chen, S., Bagdasarian, M., Kaufman, M. & Walker, E. Characterization of strong promoters from an environmental Flavobacterium hibernum strain by using a green fluorescent protein-based reporter system. Appl. Environ. Microbiol. 73, 1089-1100 (2007); Gómez, E., Álvarez, B., Duchaud, E. & Guijarro, J. A. Development of a markerless deletion system for the fish-pathogenic bacterium Flavobacterium psychrophilum. PLoS One 10, e0117969 (2015); Accetto, T. & AvgAtin, G. Inability of Prevotella bryantii to form a functional Shine-Dalgarno interaction reflects unique evolution of ribosome binding sites in Bacteroidetes. PloS one 6 (2011)).

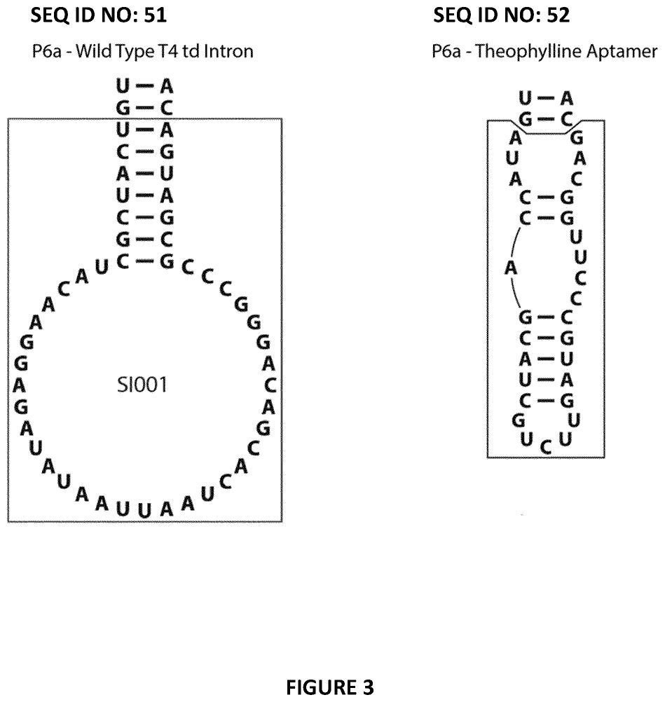

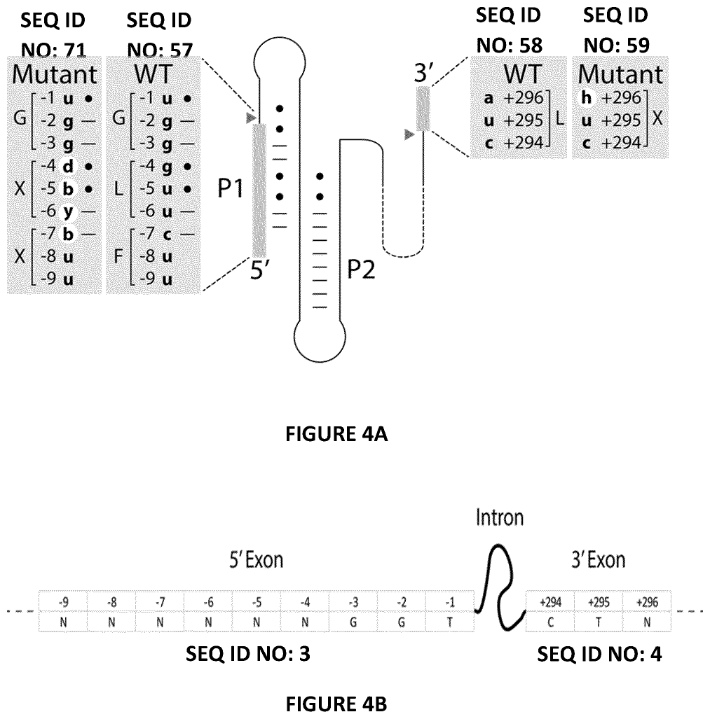

The inventors substituted the Wild Type (VVT) P6a loop of the T4 td intron with a theophylline responsive aptamer (see FIG. 3) to inducibly control the translation of the thymidylate synthase gene with a variation of theophylline responsive aptamers. The intron described by Thompson et al. (2002) Supra cannot be transferred to other genes (other than the td gene) as that would cause disruption of the amino acid sequence of the POI. Disruption of the POI is caused mainly because of the 5′ and 3′ exon sequences of the intron as they are part of the intron but they are retained in the mRNA after splicing (see FIG. 4A). Transferring the WT T4 td intron to another gene will disrupt the amino acid sequence of the encoded protein, leading to a non-functional protein. Therefore, such riboswitch is not universal and it is restricted to the td gene.

To create a universal T4 td intron riboswitch, the inventors introduce modifications to the intron allowing it to be transferred to any gene of interest without compromising its splicing activity. The modifications are located in the 5′ and 3′ exon sequences of the T4 td intron (FIGS. 4A and 4B). LacZ colorimetric assays were performed in Escherichia coli. A simplified construction system was then used in the form of “Tagged-intron” variants to control the targeting activity of FnCas12a in E. coli. The system was then transferred into industrially relevant bacterial strains of Pseudomonas putida and Flavobacterium IR1.

When converting the T4 td intron into a universal riboswitch, certain modifications were introduced to the intron, allowing it to be transferred into any gene of interest without compromising its activity. The use of the inducer controlled self-splicing intron to control CRISPR-Cas proteins was found to solve the problem of how to engineer some prokaryotes which have proved intractable previously to attempts to modify them with a Crispr-Cas approach, as previous attempts failed to do so (e.g. Flavobacterium IR1).

In more detail, the inventors explored the role of the 5′ exon and 3′ exon sequences of the td intron and determined its splicing activity by substituting the relevant bases in the 5′ exon and 3′ exon (see FIGS. 4A and 4B). Referring to FIG. 4B, this shows all possible nucleotide sequences at the 5′ and 3′ exon sequences of the T4 td intron, wherein “N” represents any nucleotide A, T, G or C. Then −9 to −1 represent the 5′ exon sequence of the T4 td intron. The +294 to +296 positions represent the 3′ exon sequence of the T4 td intron. There are 16,777,216 possibly combinations of nucleic acid changes at the −9 to −1 plus +294 to +296 positions. Of the 5′ exon sequence, positions −3 to −1 are kept wild type to maintain the self-splicing ability of the intron. Of the 3′ exon sequence, positions +294 and +295 are kept wild type to maintain the self-splicing ability of the intron.

Initially the inventors substituted the −7 and +296 positions of the 5′ exon and 3′ exon, respectively, and by inserting the different variants into the LacZa gene and by performing assays in E. coli (see Examples 1 to 3). The, positions −6, −5 and −4 of the 5′ exon of the td intron were tested. This defined several base substitutions which either allowed more self-splicing and therefore more LacZa activity, or less self-splicing and therefore less LacZa activity.

The inventors then further modified the 5′exon and 3′exon sequences of the intron in order to control/titrate its self-splicing activity, or to introduce it in multiple sites in the Open Reading Frame (ORF) of the Gene Of Interest (GOI). The inventors were successful in transferring the self-splicing intron to any GOI at different positions in the ORF.

Altered splicing efficiency by changing the base pair interactions at the P1 stem of the T4 td intron was previously observed by Pichler A. & Schroeder R. (2002) “Folding Problems of the 5′ Splice Site Containing the P1 Stem of the Group I Thymidylate Synthase Intron” J. Biol. Chem 277 (20) 17987-17993, who created two mutant variants to either stabilize (−4A, −5C, −6T) or destabilize (−4C, −5A, −6C) the base pair interactions at the P1 stem and noticed increased splicing efficiency for both the stabilized and the destabilized variants compared to the WT intron. However, these results are contradicting to the present results, as stabilization (−4A, −5C, −6T) of the P1 stem decreased the splicing efficiency by approximately 80% (compared to the WT intron) in our setup (FIG. 8D). Moreover, although we do not have generated an exact replica of the destabilized variant from Pichler et al. (2002), we do have a destabilized P1 stem with mismatches at −4 (T), −5 (G) and −6 (C). Similarly, a decrease (approximately 30%) in splicing efficiency compared to the WT intron was observed in our setup. The observed differences in splicing efficiency by stabilizing or destabilizing the P1 stem may be attributed to the different experimental setup as we investigated splicing efficiency based on enzymatic activities whereas Pichler et al. (2002), performed cis splicing assays by isolating total RNA from E. coli cells carrying the different intron variants. It is, therefore, very likely that the total RNA may give a wrong impression of the total protein concentration generated by the spliced T4 td intron variants. In addition, the translocation of the T4 td intron directly after the ATG start codon, may have affected the splicing efficiency of the intron in an unpredictable way. Our outcome is surprising and novel compared to Pichler et al. (2002), as they report contradicting results to our results and as the position of the intron is different.

The inventors further successfully provide a universal “TAG” sequence whereby the intron is introduced just after the ATG “start” codon and therefore is gene/protein independent. The TAG sequence leaves a 4 amino acid tag at the N-terminus of the protein of interest (P01) just after the methionine (m) encoded by the start codon. This tag sequence does not usually hinder the activity of the expressed protein as it consists only of 4 amino acids. A cleavage sequence of a TEV protease cleavage site can be added directly after the “Tag” sequence and then cleaved with proteases afterwards. The cleavage leaves a single amino acid attached to the protein of interest. Other cleavage sequences and proteases well known in the art may be used, e.g. https://web.expasy.org/peptide_cutter/ and https://web.expasy.org/peptide_cutter/peptidecutter_enzymes.html.

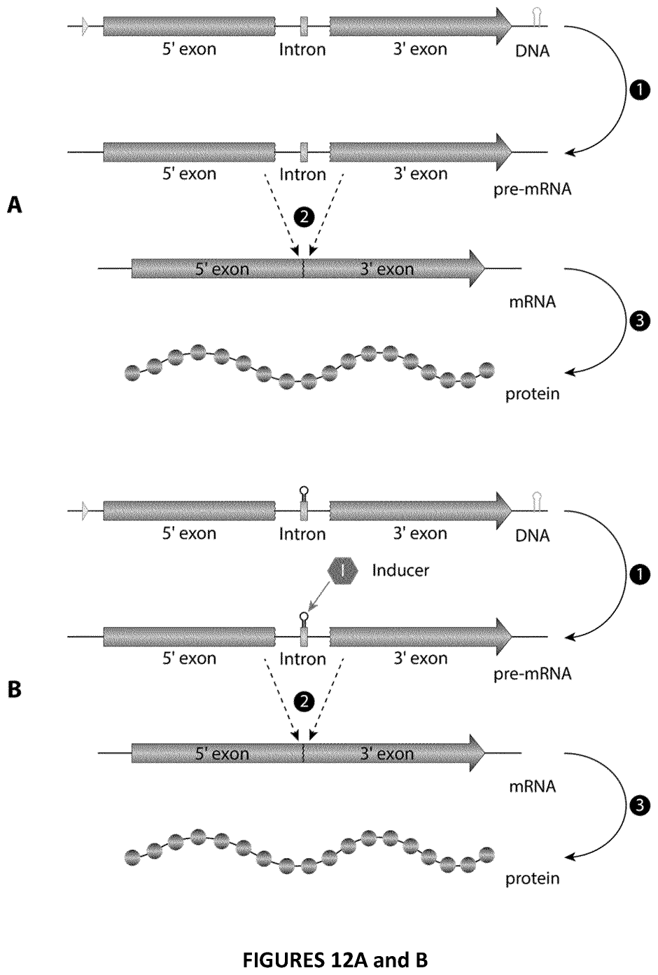

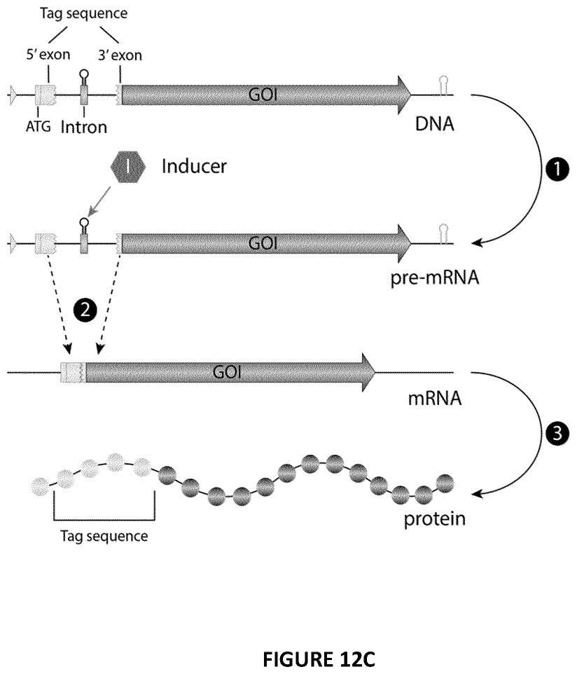

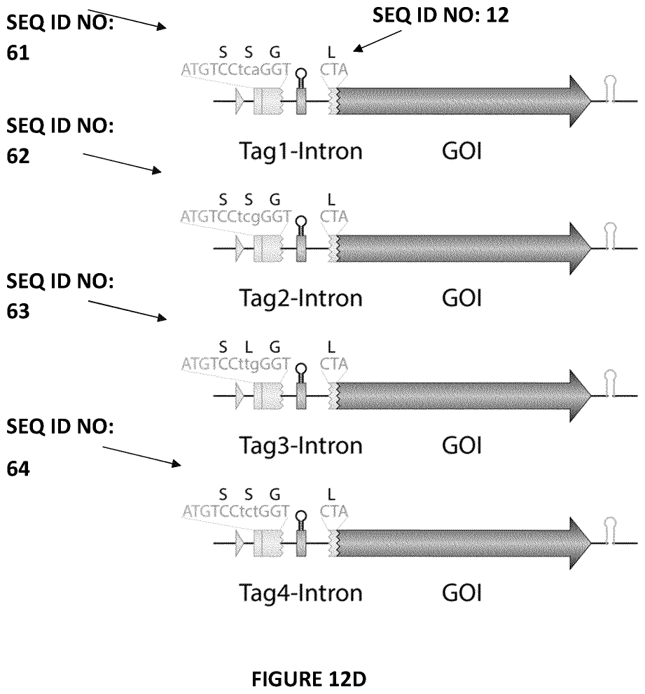

Using different versions of tag-introns, the inventors are able to control expression of a GOI at the protein level which gives the advantage of titration. Tag sequences are chosen from those shown in FIG. 12D. Tag1: −4P, −5P, −6P; Tag2: −4W, −5P, −6P; Tag3: −4W, −5W, −6P; Tag4: −4M, −5P, −6P. What the inventors have found is that self-splicing activity was in the following order: Tag1>Tag2>Tag3>Tag4, where Tag1 is the most “tight” and Tag4 is the most “loose” intron. Schematic representations of the tags are shown in FIG. 12.

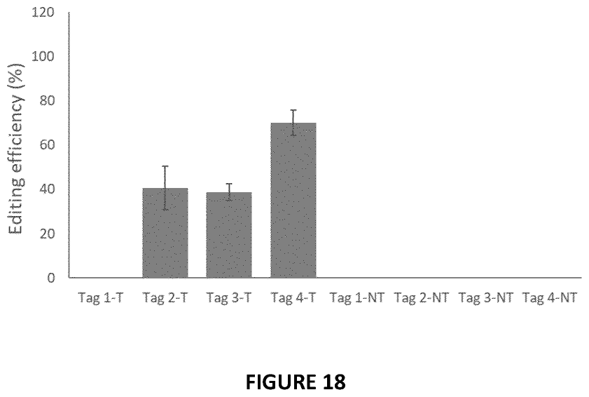

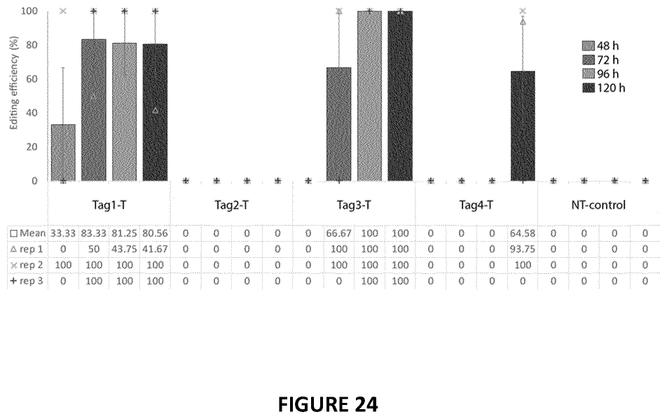

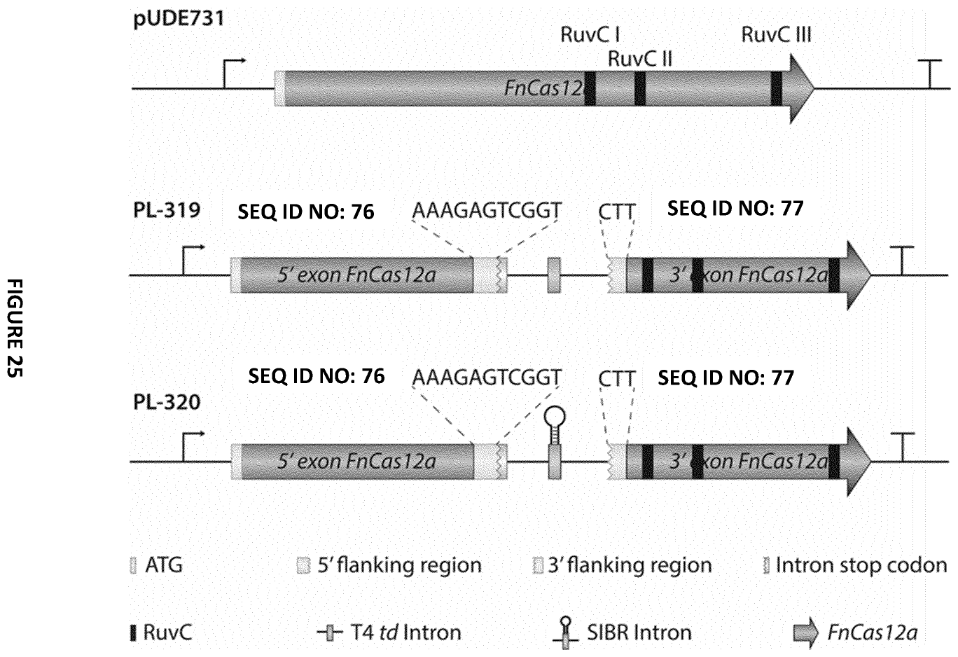

The addition of Tags has been successfully tested in E. coli, P. putida and Flavobacterium IR1 by inserting Tagged introns after the start codon of Cas12a. This approach allowed efficient editing of the bacterium of interest. More specifically, for P. putida editing efficiencies of up to 75% were reached with Tag4 (FIG. 18). In addition, the non-model Flavobacterium IR1, which was never edited before with CRISPR-Cas, was easily and efficiently engineered with efficiencies reaching 100% (FIG. 24).

The invention is applicable to any self-splicing intron and these are found in many species of bacteriophage, bacteria, protozoa and fungi, for example. The self-splicing introns are usually found embedded in specific genes of a species or strain. For example, the T4 td self-splicing intron is located in the td gene of the T4 bacteriophage.

Other self-splicing introns from bacteriophages are: T6: td, RB3: td, LZ2: td, TulA: td, ϕ1: DNA polymerase, W31: DNA polymerase, Pf-WMP3: DNA polymerase, 822: td, SPO1: DNA polymerase, SP82: DNA polymerase, cpe: DNA polymerase, SPb prophage (Ribonucleotide reductase (bnrdE and bnrdF)), Sb3: lysin, rlt: ORF40, LLH: Terminase, Twort (introns nrdE-11 & nrdE-12): ORF142.

Examples of self-splicing introns from bacteria are: Agrobacterum tumefaciens A136: tRNA^{Arg}CCU, Azoarcus sp. strain BH72: tRNA^{Ile}CAU, Coxiella burnetii (Cbu.L1917): 23S rRNA, Coxiella burnetii (Cbu.L1951): 23S rRNA, Thermotoga neapolitana NS-E Tna.bL1931: 23S rRNA, Thermotoga subterranea SL1 Tsu.bL1926: 23S rRNA, Clostridium botulinum: tmma pos. 338, Geobacillus stearothermophilus (NBRC 12550): flagellin, Bacillus sp. Kps3: flagellin, Clostridium difficile strain 630: CD3246, Anabaena PCC7120: tRN^{Leu}UAA, Scytonema hofmanii: RNA^{fMet}, Synechocystis PCC 6803: RNA^{fMet}, Neochloris aquatica: ml pos. 1931, Calothrix sp. strain PCC7601: Cal.x1, Calothrix sp. strain PCC7101: Cal.x2, L. lactis ML3: LI.LtrB, L. lactis 712: IntL, S. meliloti GR4: RmInt1.

Examples of self-splicing introns from Protozoa are: Tetrahymena thermophila (Tth.L1925): 26S rRNA, Didymium iridis (Dir.S956-1): SSU rDNA, Didymium iridis (Dir.S956-2): SSU rDNA, Physarum polycephalum (Ppo.L1925): LSU rDNA, Amoebidium parasiticum: ml, pos. 2500 and ml, pos. 1403, Naegleria (NaGIR1 and NaGIR2): SSU rRNA.

Examples of self-splicing introns from Fungi are: Neurospora crassa: ml, pos. 2449, Saccharomyces cerevisae (Sc.OX1,3): SSU rDNA, Candida albicans: 25S rRNA, Scytalidium dimidiatum (rns, pos. 1199).

Examples of self-splicing introns from other miscellaneous organisms are: Simkania negevensis Z^{T}: 23S rRNA, Chlamydomonas nivalis: rnl, pos 2593, Dunaliella parva: rnl, pos. 1931, Aureoumbra lagunensis: SSU rRNA, Bangia atropurpurea: SSU rRNA.

Calothrix sp. strain PCC7601: Cal.x1, Calothrix sp. strain PCC7101: Cal.x2, L. lactis ML3: LI.LtrB, L. lactis 712: IntL, S. meliloti GR4: RmInt1 are Group II introns, while all others are Group I introns.

Examples of Group III introns include the Euglena gracilis introns found in the psbC, rps18, ycf8, ycf13, rpoCl, rp116, psbF, rps3, rp123, rps18, rps19, rp114, rps8, rps14, rp116, psbK genes.

A unique type of ribozymes includes the self-splicing Group I introns. Group I introns have been described to control gene expression and RNA processing in bacteria and phages but also in some eukaryotes (protozoa and plants) (Hausner, G., Hafez, M. & Edgell, D. R. Bacterial group I introns: mobile RNA catalysts. Mobile DNA 5, 1-12 (2014); Edgell, D. R., Belfort, M. & Shub, D. A. Barriers to intron promiscuity in bacteria. Journal of Bacteriology 182, 5281-5289 (2000); Nielsen, H. & Johansen, S. D. Group I introns: moving in new directions. RNA biology 6, 375-383 (2009)). Due to their prevalence and simplistic nature, Group I introns have the potential to be used as universal, synthetic ribozymes to control gene expression. Especially when ribozymes are associated with a specific ligand-binding sequence (RNA aptamer), the presence/absence of such a ligand allows for switching ON/OFF the splicing activity (riboswitch), potentially controlling the expression of an associated gene. An example of a natural Group I intron-based riboswitch has been discovered in the bacterium Clostridium difficile, where its sequence resides between the RBS and the ATG start codon of an adjacent gene. After transcription, this results in a secondary structure in the 5′-UTR that prevents recruitment of the ribosome, hence hampering translation initiation. After induction by intracellular GTP or c-di-GMP, this ribozyme induces its splicing from the precursor transcript, resulting in appropriate re-positioning of the RBS upstream the start codon, thereby allowing for the ribosome to start the translation process (Lee, E. R., Baker, J. L., Weinberg, Z., Sudarsan, N. & Breaker, R. R. An allosteric self-splicing ribozyme triggered by a bacterial second messenger. Science 329, 845-848 (2010); Chen, A. G., Sudarsan, N. & Breaker, R. R. Mechanism for gene control by a natural allosteric group I ribozyme. Rna 17, 1967-1972 (2011)). Although this natural mechanism is a beautiful case of gene expression control, its requirement for specific endogenous inducers (GTP and c-di-GMP) as well as its dependency on specific secondary structures (including both the ribozyme and the coding sequence) complicates its general applicability. A synthetic alternative was provided by Thompson et al. (2002), when they combined the self-splicing Group I intron of the T4 bacteriophage with a theophylline aptamer towards a functional inducible gene expression system (Thompson, K. M., Syrett, H. A., Knudsen, S. M. & Ellington, A. D. Group I aptazymes as genetic regulatory switches. BMC biotechnology 2, 21 (2002)). Although this system was restricted to controlling the original thymidylate synthase (td) gene, we here describe its repurposing as a generic system to tune gene expression.

The inventors have also created a novel system termed Self-splicing Intron Based Riboswitch Cas (SIBR-Cas). This is created using the Group I-based aptazyme to enhance recombination in prokaryotes. The inducer controlled T4 td intron (containing an in-frame stop codon) is inserted into a CRISPR-Cas nuclease gene (Cas12a, for example) resulting in incomplete translation and avoiding formation of a functional CRISPR-Cas nuclease. Then, upon exposure to theophylline, this triggers the induction of a conformational change in the synthetic riboswitch which induces the self-splicing activity of the td intron resulting in the excision of the intron and the joining of the 5′ exon to the 3′ exon. This restores the complete mRNA of the CRISPR-Cas gene which consequently leads to the functional expression/translation of the CRISPR-Cas nuclease. In the particular example of the Cas12a protein, by controlling the expression, a time series can be made to find the appropriate induction time for counter-selection by Cas12a, thereby increasing the chances of generating correct HDR-based mutants.

So long as the relevant inducer, e.g. theophylline, can reach the self-splicing intron, then the SIBR-Cas system can be used in any organism. The advantages of such a technology are:
Tight control of the GOI (in this case the Cas protein) at the mRNA level. Complete, functional protein will be formed after the induction with theophylline
Universality—the intron can be introduced to virtually any GOI, in any archaeon, bacterium or eukaryote as long as the inducer can enter the cell of interest, at least at moderate temperatures
No complex design is required as a “tag” sequence can be used for the insertion of the intron at the beginning of the GOI
The only option for engineering non-model organisms with high AT %, low HDR efficiencies and no inducible (or characterised) promoters (see example of Flavobacterium IR1 below)

The SIBR-Cas tool can be applied for editing virtually any GOI in any cell of interest. The inventors have applied SIBR-Cas to Flavobacterium IR1.

Suitable nucleases to be used in the methods described herein are selectable at the option of the skilled person. A choice may depend upon the optimal growth temperature of the particular microbe being used. The CRISPR-Cas nucleases may be selected from any Cas Type I, Type II or Type III. More particularly, the Cas may be selected from Cas9, Cas12a (previously known as Cpf1) or Cas13 (previously known as C2c2); also any of Caw, Cas12b, Cas12c, Cas13a,b,c,d, Cas4, Csn2, Csf1, Csx10, Csx11, Cmr5, Csm2, Cas10, Csy1,2,3, Cse1,2, Cas10d, Cas8a,b,c, Cas5 or Cas3. The CRISPR-Cas nucleases may any variant from any species, whether well-known, e.g. from Streptococcus pyogenes (SpyCas9), or less commonly used such as from Geobacillus thermodenitrificans T12 (ThermoCas9) or Geobacillus stearothermophilus (GeoCas9). Methods described herein may preferably use Cas9, preferably Streptococcus pyogenes Cas9; or C2c1. Alternatively, methods described herein may preferably use Cas 12a (Cpf1). Further alternative nucleases suitable for the methods described herein are C2C3 or Argonaute. It is also contemplated that the methods described herein may use other nucleases such as zinc finger nucleases (ZFNS), meganucleases or transcription activator effector like nucleases (TALENS

In order that expression of any of the polynucleotide constructs or expression vectors of the invention described herein can be carried out in a chosen host cell, the these incorporate regulatory elements which allow expression in the host cell of interest and preferably which facilitate high-levels of expression. Such regulatory sequences may be capable of influencing transcription or translation of a gene or gene product, for example in terms of initiation, accuracy, rate, stability, downstream processing and mobility.

Such elements may include, for example, strong and/or constitutive promoters, 5′ and 3′ UTR's, transcriptional and/or translational enhancers, transcription factor or protein binding sequences, start sites and termination sequences, ribosome binding sites, recombination sites, polyadenylation sequences, sense or antisense sequences, sequences ensuring correct initiation of transcription and optionally poly-A signals ensuring termination of transcription and transcript stabilisation in the host cell. The regulatory sequences may be plant-, animal-. bacteria-, fungal- or virus derived, and preferably may be derived from the same organism as the host cell. Clearly, appropriate regulatory elements will vary according to the host cell of interest. For example, regulatory elements which facilitate high-level expression in prokaryotic host cells such as in E. coli may include the pLac, T7, P(Bla), P(Cat), P(Kat), trp or tac promoters. Regulatory elements which facilitate high-level expression in eukaryotic host cells might include the AOX1 or GAL1 promoter in yeast or the CMV- or SV40-promoters, CMV-enhancer, SV40-enhancer, Herpes simplex virus VIP16 transcriptional activator or inclusion of a globin intron in animal cells. In plants, constitutive high-level expression may be obtained using, for example, the Zea mays ubiquitin 1 promoter or 35S and 19S promoters of cauliflower mosaic virus.

Suitable regulatory elements may be constitutive, whereby they direct expression under most environmental conditions or developmental stages, developmental stage specific or inducible. Suitably, promoters may be chosen which permit expression of the protein of interest at particular developmental stages or in response to extra- or intra-cellular conditions, signals or externally applied stimuli. For example, a range of promoters exist for use in E. coli which give high-level expression at particular stages of growth (e.g. osmY stationary phase promoter) or in response to particular stimuli (e.g. HtpG Heat Shock Promoter).

Suitable expression vectors may comprise additional sequences encoding selectable markers which allow for the selection of said vector in a suitable host cell and/or under particular conditions.

Regarding transformation of a host cell with an heterologous gene sequence, expression constructs comprising the polynucleotide sequences of the invention may be located in plasmids (expression vectors) which are used to transform the host cell. Methods of transformation may include but are not limited to; heat shock, electroporation, particle bombardment, chemical induction, microinjection and viral transformation, Agrobacterium-mediated transformation, PEG-mediated transformation, lipofection.

As well as a ROI or POI, the polynucleotides of the invention as described herein may include a selectable marker protein. This may be used to screen cell populations positively or negatively. For example, the expression of a particular POI in a host cell may be coupled to relief of an auxotrophic deficit, it will be appreciated that such selectable markers may include polynucleotide sequences encoding proteins to which the cell is fatally sensitive. In these embodiments of the invention, the presence of the desired product may be coupled to the restoration of translation of the reporter protein. In this way host cells expressing the protein of interest may be selected from those which do not express the protein of interest.

Where the expression of a particular POI in a host cell is coupled to promotion of cell growth and/or division, it will be appreciated that such selectable markers may include polynucleotide sequences encoding proteins which promote cell growth and/or division. In these embodiments of the invention, the presence of the desired product may be coupled to the restoration of translation of the reporter protein. In this way host cells expressing the protein of interest may be selected from those which do not express the protein of interest.

The polynucleotides may include a reporter protein which may be assayed for or monitored for. Such reporter proteins include for example Green Fluorescent Protein (GFP), Yellow Fluorescent Protein (YFP), Red Fluorescent Protein (RFP), Cyan Fluorescent Protein (CFP), or Luciferase fusion tags. The reporter protein may be an enzyme which can be used to generate an optical signal. Alternatively, the expression vector may incorporate a polynucleotide reporter encoding a luminescent protein, such as a luciferase (e.g. firefly luciferase). Alternatively, the reporter gene may be a chromogenic enzyme which can be used to generate an optical signal, e.g. a chromogenic enzyme (such as beta-galactosidase (LacZ) or beta-glucuronidase (Gus)).

Tags used for detection of reporter protein expression may also be antigen peptide tags. A cleavable tag may also be provided for affinity purification, e.g. a polyhistidine tag. It is envisaged that other types of label may also be used to indicate expression of the reporter protein including, for example, organic dye molecules or radiolabels. In particular, preferred expression vectors will include sequences encoding a fluorescent protein, for example GFP which will enable the screening and optionally separation (selection) of a cell which expresses the protein of interest for example by Fluorescence Activated Cell Sorting (FACS).

### EXAMPLES

### Example 1: Effect of Position −7 on the Self-Splicing of the T4 td Intron

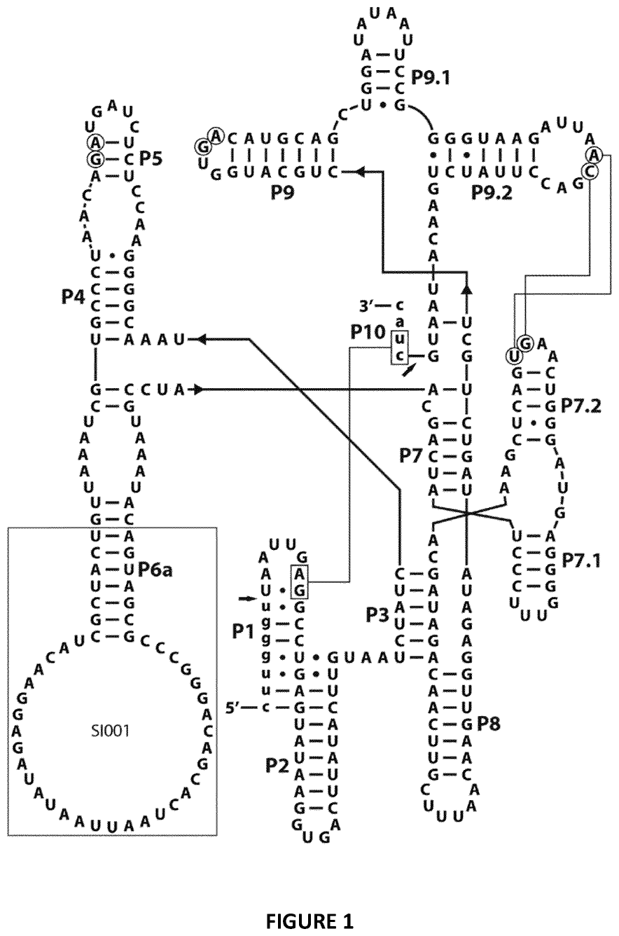

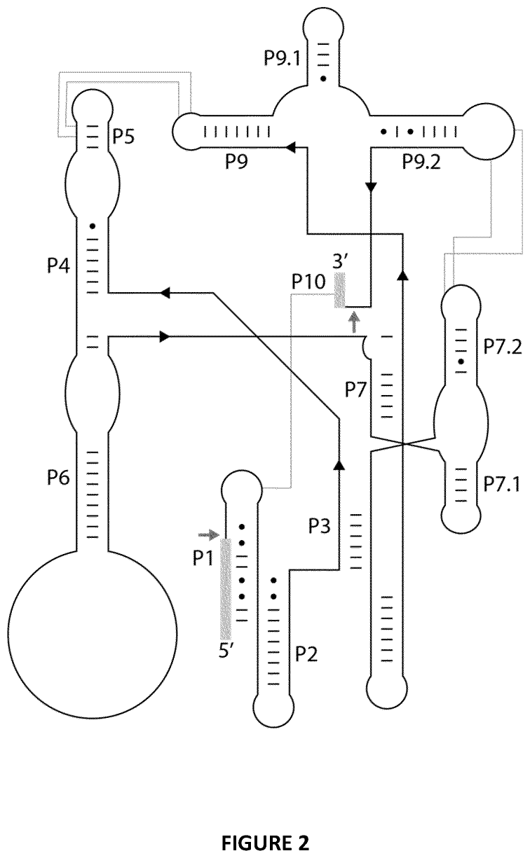

The flanking regions (5′ and 3′ exons) of the group I introns are part of the coding sequence as well as of the ribozyme (see FIGS. 1, 2 and 4). The T4 td intron structure as shown in the figures follows the format of Cech, T. R., et al., (1994) “Representation of the secondary and tertiary structure of group I introns” Nature Structural Biology 1 (5): 273-280. Uppercase letters indicate the intron, lowercase letters the exons. Arrows indicate the splice site. Boxed portions can be replaced by the theophylline aptamer to generate a theophylline-dependent aptazyme. Referring to FIG. 2, the intron loops are shown as P1 to P10. The light grey boxes indicate the 5′ and 3′ exon interactions with the intron. Horizontal or vertical lines within the loops indicate base pairing, whereas the black solid circles within the loops indicate wobble pairing. Light grey arrows indicate the splice site. FIG. 3 shows wild type and theophylline aptamer structures of the P6a loop of the T4 td intron. Left hand portion of FIG. 3 is the Wild Type (WT) P6a loop of the T4 td intron. Right hand portion of FIG. 3 is the theophylline aptamer which replaces the WT P6a loop for inducible splicing of the intron.

When inserting the intron into another gene it is almost impossible to retain both the intron flanking regions and the CDS. Applying minor changes to the CDS with synonymous codons may create a site that resembles the wild type intron flanking regions. However, it is not clear to which extent the flanking regions determine the splicing efficiency.

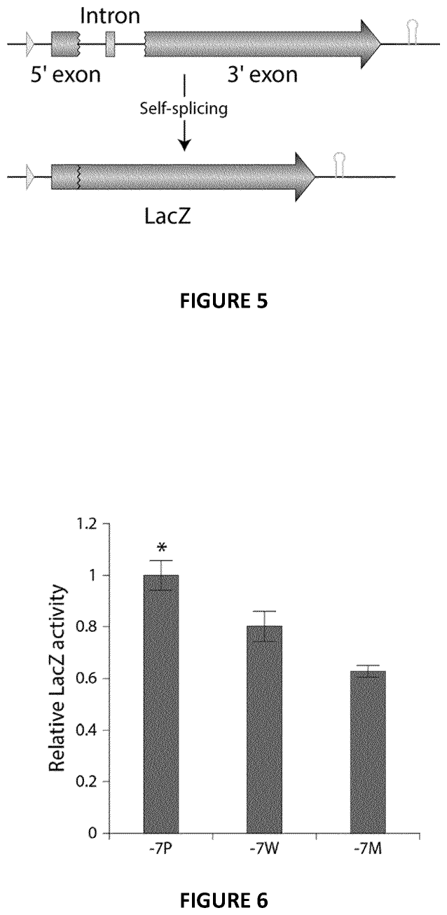

To investigate the effect of the flanking regions of the T4 td intron on its splicing efficiency and on the expression of the target gene, a series of constructs were made containing the lacZ gene from E. coli with the intron in between amino acids D6 and S7 (see FIG. 5). These amino acids were identified because insertion at this location would be least likely to impact on the structure of the protein. The lacZ gene was used because its functional expression can be easily monitored by a colorimetric assay and because of its high tolerance for modification at the 5′ end. LacZa is interrupted with the T4 td intron and therefore non-functional. Upon self-splicing and excision of the intron, the ORF of LacZa is complete and functional. LacZa encodes for β-galactosidase which is able to hydrolyse ortho-nitrophenyl-β-D-galactopyranoside (ONPG) into β-D-galactose and ortho-nitrophenol (ONP). ONP can be measured through colorimetric assays. Mutations were made by PCR in the 5′ flank as depicted in Table 1.

TABLE 1
Primers used to introduce point mutations at the −7 position of the 5′ exon
of the T4 td intron. Bold bases show the −7 to −4 positions. Underlined bases show
the −7 point mutations.

| Plasmid name | −7 | −6 | −5 | −4 | +296 | Forward | Reverse |
|---|---|---|---|---|---|---|---|
| PEA001 [WT] | P | P | W | W | M | GATCTTAAGGATG | TGActgcagAATATTAA |
| | | | | | | TTCT [symbol] GGTTAAT | ACGGTAGCATTATGT |
| | | | | | | TGAGGCCTGAGTA | TCAGATAAGGTCG |
| | | | | | | TAAGGTG (SEQ ID | (SEQ ID NO: 25) |
| | | | | | | NO: 24) | |
| pEA001 [−7W] | W | P | W | W | M | GATCTTAAGGATG | TGActgcagAATATTAA |
| | | | | | | TTCT [symbol] GGTTAAT | ACGGTAGCATTATGT |
| | | | | | | TGAGGCCTGAGTA | TCAGATAAGGTCG |
| | | | | | | TAAGGTG (SEQ ID | (SEQ ID NO: 25) |
| | | | | | | NO: 26) | |
| pEA001 [−7M] | M | P | W | W | M | GATCTTAAGGATG | TGActgcagAATATTAA |
| | | | | | | TTTT [symbol] GGTTAAT | ACGGTAGCATTATGT |
| | | | | | | TGAGGCCTGAGTA | TCAGATAAGGTCG |
| | | | | | | TAAGGTG (SEQ ID | (SEQ ID NO: 25) |
| | | | | | | NO: 27) | |

P: Pair; W: Wobble; M: Mismatch.

The wild type interactions are shown in FIG. 4A. All position numbers are relative to the 5′ splice site. The P1 suggested by Thompson et al. (2002) Supra does not show involvement of position −7 in base pairing. Since there is the possibility of base pairing, the effect of disallowing base pairing was assessed. If there is no interaction between position −7 and the Internal Guide Sequence (IGS), (the sequence at P1 that forms the base pairing with the 5′ exon sequence), and no difference in LacZ activity should be observed. The −7 position was mutated (while all other nucleotides remained WT) to form either a wobble pair or a mismatch. The self-splicing activity of the three intron variants for β-galactosidase activity was assessed after overnight growth.

In more detail in FIG. 4A, the triangles indicate the splicing site. The light grey boxes attached to the intron indicate the 5′ and 3′ exons. WT and mutant variants are represented for both 5′ and 3′ exons. The white circles indicate modified nucleotides. Nucleotides have been modified into “b” (G/U/C), “y” (C/U), “d” (G/NU) or “h” (A/U/C). Positions −7 and +296 were changed in single nucleotide mutants only where the other positions conformed to the natural intron. Positions −4 to −6 were changed in all possible combinations of match, mismatch and wobble (where possible).

FIG. 6 shows how changing WT −7 position (pair) to a wobble pair or mismatch leads to a decreased β-galactosidase activity which also indicates decreased splicing activity of the mutant T4 td introns. Changes at the −7 position negatively affect the intron splicing. Both the wobble pair (−7W) and the mismatch (−7M) show a decreased activity compared to the wild type (−7P). The results show that position −7 preferably pairs with position +15. A weaker interaction in the form of a wobble base pair does impede the intron splicing, but not as severely as having no interaction at all.

### Example 2: Effect of Position +296 on the Self-Splicing of the T4 td Intron

Thompson et al. (2002) Supra do not show any interaction between position +296 (the +3 position in the 3′ exon) and the P1 loop of the T4 td intron. This is similar to the situation with the −7 position. Therefore, point mutations at the +296 position of the T4 td intron were made to see if they might impact on the splicing activity of the intron. The WT +296 position (mismatch) was mutated by PCR (Table 2) to form either a pair or a wobble pair with the P1 loop. All mutants were assayed for β-galactosidase activity after overnight growth.

TABLE 2
primers used to introduce point mutations at the +296 position of the 3′ exon of the
T4 td intron. Bold underlined bases show the +296 point mutations. Sequences are shown
from 5′ to 3′.

| Plasmid name | −7 | −6 | −5 | −4 | +296 | Forward | Reverse |
|---|---|---|---|---|---|---|---|
| pEA001 [WT] | P | P | W | W | M | GATCTTAAGGA | TGActgcagAATATTAA |
| | | | | | | TGTTCTcttgGGT | ACGG [symbol] AGCATTATGTT |
| | | | | | | TAATTGAGGCC | CAGATAAGGTCG |
| | | | | | | TGAGTATAAGG | (SEQ ID NO: 25) |
| | | | | | | TG (SEQ ID NO: | |
| | | | | | | 24) | |
| pEA001 [296P] | P | P | W | W | P | GATCTTAAGGA | TGActgcagAATATTAA |
| | | | | | | TGTTTTcttgGGT | ACGG [symbol] AGCATTATGT |
| | | | | | | TAATTGAGGCC | TCAGATAAGGTCG |
| | | | | | | TGAGTATAAGG | (SEQ ID NO: 29) |
| | | | | | | TG (SEQ ID NO: | |
| | | | | | | 28) | |
| pEA001 [296W] | P | P | W | W | W | GATCTTAAGGA | TGActgcagAATATTAA |
| | | | | | | TGTTTTcttgGGT | ACGG [symbol] AGCATTATGT |
| | | | | | | TAATTGAGGCC | TCAGATAAGGTCG |
| | | | | | | TGAGTATAAGG | (SEQ ID NO: 30) |
| | | | | | | TG (SEQ ID NO: | |
| | | | | | | 28) | |

P: Pair; W: Wobble; M: Mismatch.

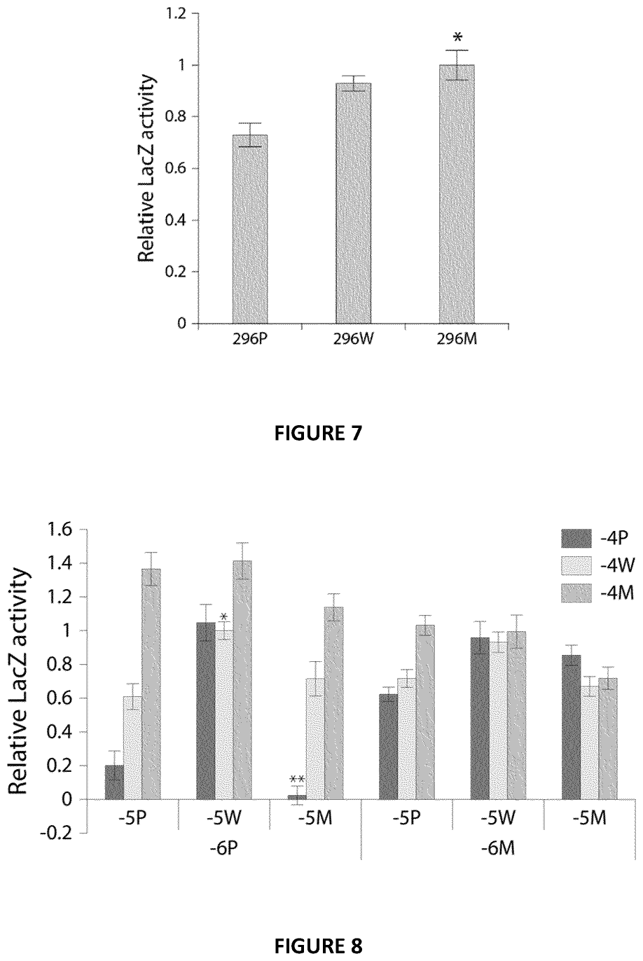

FIG. 7 shows the results of LacZ activity of position +296 mutants. The asterisk indicates the wild-type intron. Base substitution at the +296 position led to reduced 3-galactosidase activity. Stabilising the interactions between the 5′ end of the intron and the 5′ end of the 3′ exon does not aid the splicing as both the pair (+296P) and the wobble pair (+296W) exhibit a lower LacZ activity than the wild type (+296M). The observed reduction implies that a mismatch allows for the highest intron splicing activity whereas the weak wobble base pair impedes splicing to some extent and the stronger pair decreases the splicing to a significantly larger extent. Therefore, the WT position +296 of T4 td intron does not appear to involve pairing with the P1 loop and that alterations in the +296 position pairing impede the self-splicing activity of the T4 td intron.

### Example 3: Effect of Positions −4 to −6 on the Self-Splicing of the T4 td Intron

This investigated the effect of altering positions −4 to −6 in all possible combinations of pair (P), mismatch (M) and wobble pair (W) (if applicable) whilst preserving all the other bases as WT. With reference to FIGS. 1 and 4A, positions −4 to −6 is GUU (positions −6 to −4 are UUG) and these pair or interact with UGA at positions +12 to +14, respectively. Mutations where introduced by PCR as indicated in Table 3.

In FIG. 8 the wild type intron (*) is (sequences shown from 5′ to 3′) PWM/(UUG) and set to 1. All other LacZ activities are a fraction of the wild type activity. PMP reads either UAA or UGA both being stop codons (**). FIG. 8 shows that a −4 mismatch is preferred in almost all variants, except for those in which both −6 and −5 are mismatched too. A wobble base pair at position −5 negates to a large extent the effect that −6 and −4 have on the splicing. In contrast, a pair or a mismatch at position −5 means that depending on −6 and −4 the splicing efficiency may be very high or very low. Position −6 in general appears in favour of being paired, however, strengthening the P1 to full extent (PPP) is detrimental for the splicing. Completely mismatching positions −6 to −4 impedes the splicing but not to a very large extent. The cumulative effect of changes in the intron flanking regions remains currently unknown, but for many genes an insertion position with at least decent splicing can be found already by retaining the WT interactions of positions −1 to −3, and 294 to 295 and possibly changing positions −4 to −6. Such alterations will yield an active intron with a good splicing efficiency.

TABLE 3
Primers used to introduce mutations at the −6 to −4 positions of the 5′ exon
of the T4 td intron. Bold bases show the −7 to −4 positions. Underlined bases show the −6
to −4 mutations. Sequences are shown from 5′ to 3′.

| Plasmid name | −7 | −6 | −5 | −4 | 296 | Forward | Reverse |
|---|---|---|---|---|---|---|---|
| pEA001 [WT] | P1 | P | W | W | M | GATCTTAAGGA | TGActgcagAATATTAA |
| | | | | | | TGTTCT [symbol] GG | ACGGTAGCATTATGT |
| | | | | | | TTAATTGAGGC | TCAGATAAGGTCG |
| | | | | | | CTGAGTATAAG | (SEQ ID NO: 25) |
| | | | | | | GTG (SEQ ID | |
| | | | | | | NO: 24) | |
| pEA001 [PPP] | P | P | P | P | M | GATCTTAAGGA | TGActgcagAATATTAA |
| | | | | | | TGTTTT [symbol] GG | ACGGTAGCATTATGT |
| | | | | | | TTAATTGAGGC | TCAGATAAGGTCG |
| | | | | | | CTGAGTATAAG | (SEQ ID NO: 25) |
| | | | | | | GTG (SEQ ID | |
| | | | | | | NO: 72) | |
| pEA001 [PPW] | P | P | P | W | M | GATCTTAAGGA | TGActgcagAATATTAA |
| | | | | | | TGTTTT [symbol] GG | ACGGTAGCATTATGT |
| | | | | | | TTAATTGAGGC | TCAGATAAGGTCG |
| | | | | | | CTGAGTATAAG | (SEQ ID NO: 25) |
| | | | | | | GTG (SEQ ID | |
| | | | | | | NO: 73) | |
| pEA001 [PPM] | P | P | P | M | M | GATCTTAAGGA | TGActgcagAATATTAA |
| | | | | | | TGTTTT [symbol] GG | ACGGTAGCATTATGT |
| | | | | | | TTAATTGAGGC | TCAGATAAGGTCG |
| | | | | | | CTGAGTATAAG | (SEQ ID NO: 25) |
| | | | | | | GTG (SEQ ID | |
| | | | | | | NO: 74) | |
| pEA001 [PWP] | P | P | W | P | M | GATCTTAAGGA | TGActgcagAATATTAA |
| | | | | | | TGTTTT [symbol] GG | ACGGTAGCATTATGT |
| | | | | | | TTAATTGAGGC | TCAGATAAGGTCG |
| | | | | | | CTGAGTATAAG | (SEQ ID NO: 25) |
| | | | | | | GTG (SEQ ID | |
| | | | | | | NO: 75) | |
| pEA001 [PWW] | P | P | W | W | M | GATCTTAAGGA | TGActgcagAATATTAA |
| | | | | | | TGTTTT [symbol] GG | ACGGTAGCATTATGT |
| | | | | | | TTAATTGAGGC | TCAGATAAGGTCG |
| | | | | | | CTGAGTATAAG | (SEQ ID NO: 25) |
| | | | | | | GTG (SEQ ID | |
| | | | | | | NO: 28) | |
| pEA001 [PWM] | P | P | W | M | M | GATCTTAAGGA | TGActgcagAATATTAA |
| | | | | | | TGTTTT [symbol] GGT | ACGGTAGCATTATGT |
| | | | | | | TAATTGAGGCC | TCAGATAAGGTCG |
| | | | | | | TGAGTATAAGG | (SEQ ID NO: 25) |
| | | | | | | TG (SEQ ID NO: | |
| | | | | | | 31) | |
| pEA001 [PMP] | P | P | M | P | M | GATCTTAAGGA | TGActgcagAATATTAA |
| | | | | | | TGTTTT [symbol] GG | ACGGTAGCATTATGT |
| | | | | | | TTAATTGAGGC | TCAGATAAGGTCG |
| | | | | | | CTGAGTATAAG | (SEQ ID NO: 25) |
| | | | | | | GTG (SEQ ID | |
| | | | | | | NO: 32) | |
| pEA001 [PMW] | P | P | M | W | M | GATCTTAAGGA | TGActgcagAATATTAA |
| | | | | | | TGTTTT [symbol] GG | ACGGTAGCATTATGT |
| | | | | | | TTAATTGAGGC | TCAGATAAGGTCG |
| | | | | | | CTGAGTATAAG | (SEQ ID NO: 25) |
| | | | | | | GTG (SEQ ID | |
| | | | | | | NO: 33) | |
| pEA001 [PMM] | P | P | M | M | M | GATCTTAAGGA | TGActgcagAATATTAA |
| | | | | | | TGTTTT [symbol] GG | ACGGTAGCATTATGT |
| | | | | | | TTAATTGAGGC | TCAGATAAGGTCG |
| | | | | | | CTGAGTATAAG | (SEQ ID NO: 25) |
| | | | | | | GTG (SEQ ID | |
| | | | | | | NO: 34) | |
| pEA001 [MPP] | P | M | P | P | M | GATCTTAAGGA | TGActgcagAATATTAA |
| | | | | | | TGTTTT [symbol] GG | ACGGTAGCATTATGT |
| | | | | | | TTAATTGAGGC | TCAGATAAGGTCG |
| | | | | | | CTGAGTATAAG | (SEQ ID NO: 25) |
| | | | | | | GTG (SEQ ID | |
| | | | | | | NO: 35) | |
| pEA001 [MPW] | P | M | P | W | M | GATCTTAAGGA | TGActgcagAATATTAA |
| | | | | | | TGTTTT [symbol] GG | ACGGTAGCATTATGT |
| | | | | | | TTAATTGAGGC | TCAGATAAGGTCG |
| | | | | | | CTGAGTATAAG | (SEQ ID NO: 25) |
| | | | | | | GTG (SEQ ID | |
| | | | | | | NO: 36) | |
| pEA001 [MPM] | ? | M | P | M | M | GATCTTAAGGA | TGActgcagAATATTAA |
| | | | | | | TGTTTT [symbol] GG | ACGGTAGCATTATGT |
| | | | | | | TTAATTGAGGC | TCAGATAAGGTCG |
| | | | | | | CTGAGTATAAG | (SEQ ID NO: 25) |
| | | | | | | GTG (SEQ ID | |
| | | | | | | NO: 37) | |
| pEA001 [MPW] | P | M | W | P | M | GATCTTAAGGA | TGActgcagAATATTAA |
| | | | | | | TGTTTT [symbol] GG | ACGGTAGCATTATGT |
| | | | | | | TTAATTGAGGC | TCAGATAAGGTCG |
| | | | | | | CTGAGTATAAG | (SEQ ID NO: 25) |
| | | | | | | GTG (SEQ ID | |
| | | | | | | NO: 38) | |
| pEA001 [MWW] | P | M | W | W | M | GATCTTAAGGA | TGActgcagAATATTAA |
| | | | | | | TGTTTT [symbol] GG | ACGGTAGCATTATGT |
| | | | | | | TTAATTGAGGC | TCAGATAAGGTCG |
| | | | | | | CTGAGTATAAG | (SEQ ID NO: 25) |
| | | | | | | GTG (SEQ ID | |
| | | | | | | NO: 39) | |
| pEA001 [MWM] | P | M | W | M | M | GATCTTAAGGA | TGActgcagAATATTAA |
| | | | | | | TGTTTT [symbol] GG | ACGGTAGCATTATGT |
| | | | | | | TTAATTGAGGC | TCAGATAAGGTCG |
| | | | | | | CTGAGTATAAG | (SEQ ID NO: 25) |
| | | | | | | GTG (SEQ ID | |
| | | | | | | NO: 40) | |
| pEA001 [MMP] | P | M | M | P | M | GATCTTAAGGA | TGActgcagAATATTAA |
| | | | | | | TGTTTT [symbol] GG | ACGGTAGCATTATGT |
| | | | | | | TTAATTGAGGC | TCAGATAAGGTCG |
| | | | | | | CTGAGTATAAG | (SEQ ID NO: 25) |
| | | | | | | GTG (SEQ ID | |
| | | | | | | NO: 41) | |
| pEA001 [MMW] | P | M | M | W | M | GATCTTAAGGA | TGActgcagAATATTAA |
| | | | | | | TGTTTT [symbol] GG | ACGGTAGCATTATGT |
| | | | | | | TTAATTGAGGC | TCAGATAAGGTCG |
| | | | | | | CTGAGTATAAG | (SEQ ID NO: 25) |
| | | | | | | GTG (SEQ ID | |
| | | | | | | NO: 43) | |
| pEA001 [MMM] | P | M | M | M | M | GATCTTAAGGA | TGActgcagAATATTAA |
| | | | | | | TGTTTT [symbol] GG | ACGGTAGCATTATGT |
| | | | | | | TTAATTGAGGC | TCAGATAAGGTCG |
| | | | | | | CTGAGTATAAG | (SEQ ID NO: 25) |
| | | | | | | GTG (SEQ ID | |
| | | | | | | NO: 42) | |

P: Pair; W: Wobble; M: Mismatch.

### Example 4: Script Development for the Introduction of the T4 td Intron at any Gene of Interest

Transferring the T4 td intron into the open reading frame (ORF) of genes other than the WT thymidylate synthase gene, can be achieved by following the script provided in Example 11, and by introducing silent mutations to the 5′ and 3′ flanking regions of the intron. The script retains the WT interactions of positions −1 to −3, +294 and +295, but changes the positions −4 to −6 and +296 in order to find an insertion side in the gene of interest (GOI). The script ensures that the insertion side preserves the amino acids of the encoded protein from the GOI by introducing silent mutations and it also ensures sufficient splicing activity of the intron according to our previous results.

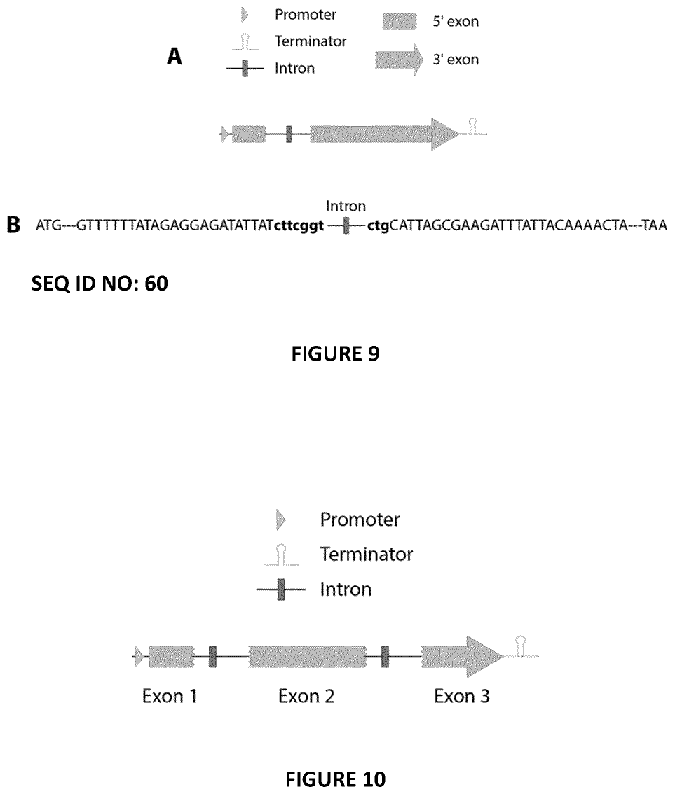

FIG. 9A shows a cartoon of the insertion of the modified T4 td intron at the GOI. FIG. 9B shows the insertion site in the WT FnCas12a gene. Bases shown in lower case and bold typeface indicate 5′ and 3′ exon sequences that are modified and interact with the T4 td intron.

An example of the insertion site for the FnCas12a gene is shown in FIG. 9B and the insertion site was generated using the script as described hereto and in Example 11. This script can be applied virtually for any GOI. In addition, multiple introns (more than one) can be introduced in the GOI as shown in FIG. 10. Multiple introns will provide a tighter control for self-splicing.

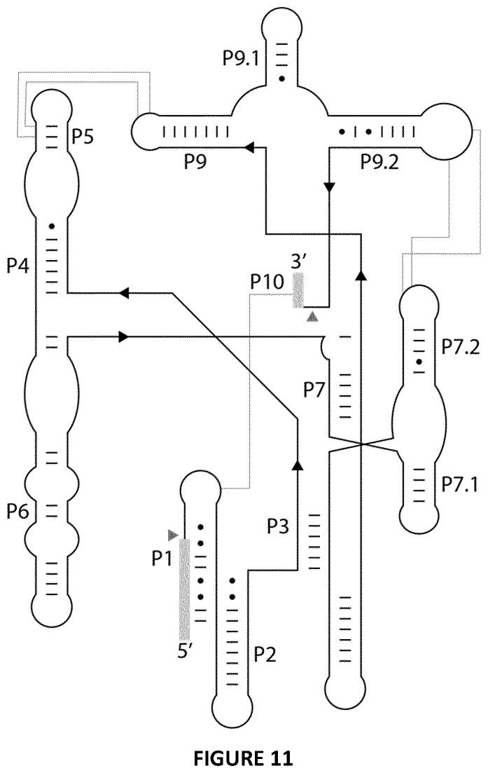

To control the splicing activity of the T4 td intron, Thompson et al. (2002) Supra attached a theophylline aptamer at the P6 stem loop of the T4 td intron. In a similar fashion, the theophylline aptamer was also added to the modified (changes at positions −4 to −6) T4 td introns developed in this example. In this way, tight, titratable and inducible control of the GOI was obtained. A schematic representation of the T4 td intron with the theophylline aptamer at the P6 stem loop is shown in FIG. 11. A step-wise cartoon is also shown at FIGS. 12A and 12B. This shows the steps of DNA to RNA to protein transcription and compares this to the steps of induction and translation of the intron-GOI constructs of the invention. In FIG. 12A the GOI is split by the WT T4 td intron. Step (1): the GOI is transcribed to form the “inactive” pre-mRNA molecule. Step (2): the intron is excised by spontaneous self-splicing events yielding a functional mRNA. Step (3): the mRNA is translated into the protein of interest. In FIG. 12B the GOI is split by the theophylline dependent T4 td intron. Splicing and formation of the mRNA occurs only in the presence of an inducer e.g. theophylline.

### Example 5: Generation of Tagged-T4 td Intron Variants and Use Thereof to Control Expression of FnCas12a in Escherichia coli MG1655

To further control the splicing activity of the modified introns, a theophylline aptamer was added at the P6 stem loop of the T4 td intron as previously described (see Thompson et al. (2002) Supra) and shown in FIG. 11. Also, to simplify the design and usage of the system, the T4 td intron was introduced at the 5′ end of the gene of interest (GOI), directly after the ATG start codon, preserving the original reading frame of the protein of interest (POI) (FIG. 12C).

As shown in FIG. 12C, the intron is inserted directly after the ATG start codon of the GOI which will result in a 4 amino acid tag sequence when not counting the M from the start codon (and a 5 amino acid tag including the M encoded by the start codon) attached to the final translated protein. Step (1): the GOI is transcribed to form the “inactive” pre-mRNA molecule. Step (2): upon ligand binding the aptamer changes conformation and the intron can splice out of the mRNA, yielding a functional mRNA. Step (3): the mRNA is translated into the protein of interest which contains a 4 amino acid tag sequence at the N-terminus.

Splicing of the intron results in a short (four amino acid long) tag sequence attached to the N-terminus of the POI (when not counting the M encoded by the start codon) whereas unspliced mRNA results in a small, non-functional peptide sequence (due to stop codons present in the T4 td intron).

FIG. 12D shows theophylline dependent T4 td introns introduced as tags after the start codon “ATG” of the gene of interest (GOI). Four different intron variants (as shown previously in FIG. 8), referred to as “Tag1”, “Tag2”, etc. (Tag1: −4P, −5P, −6P; Tag2: −4W, −Tag3: −4W, −5W, −6P; Tag4: −4M, −5P, −6P) are inserted directly after the start codon of the GOI (FnCas12a gene). The 5′ exon sequences of the four different tags are indicated: Tag1: TCCtcaGGT (SEQ ID NO: 7); Tag2: TCCtcgGGT (SEQ ID NO: 8); Tag3: TCCttgGGT (SEQ ID NO: 9); Tag4: TCCtctGGT (SEQ ID NO: 10). The 3′ exon sequence is conserved amongst the 4 different tags and indicated as CTA (SEQ ID NO: 12). The amino acids corresponding to the Tag sequences are indicated with capital bold letters above the Tagged-introns. Intron-less (wild-type) FnCas12a was used as a reference for comparison. Efficiency of targeting of the different Tagged-T4 td intron variants was assessed by using either a LacZa targeting (T) or a non-targeting (NT) crRNA and by comparing the amount of colony forming units (CFUs) of transformed E. coli MG1655 per μg of plasmid used (CFUs μg⁻¹). CFU was determined by the number of colonies observed. All the plasmids used for this experiment are listed in Table 4.

TABLE 4
Plasmids used for targeting in E. coli MG 1655.

| Plasmid name | Description and relevant characteristics |
|---|---|
| pSIBR EcoPpu NT tag 1 | KanR, FnCas12a with Tag1-intron, NT crRNA |
| pSIBR EcoPpu NT tag 2 | KanR, FnCas12a with Tag2-intron, NT crRNA |
| pSIBR EcoPpu NT tag 3 | KanR, FnCas12a with Tag3-intron, NT crRNA |
| pSIBR EcoPpu NT tag 4 | KanR, FnCas12a with Tag4-intron, NT crRNA |
| pSIBR EcoPpu NT no intron | KanR, WT FnCas12a, NT crRNA |
| pSIBR EcoPpu T lacZ tag 1 | KanR, FnCas12a with Tag1-intron, LacZ T |
| | crRNA |
| pSIBR EcoPpu T lacZ tag 2 | KanR, FnCas12a with Tag2-intron, LacZ T |
| | crRNA |
| pSIBR EcoPpu T lacZ tag 3 | KanR, FnCas12a with Tag3-intron, LacZ T |
| | crRNA |
| pSIBR EcoPpu T lacZ tag 4 | KanR, FnCas12a with Tag4-intron, LacZ T |
| | crRNA |
| pSIBR EcoPpu T lacZ no intron | KanR, WT FnCas12a, LacZ T crRNA |

Electrocompetent E. coli MG1655 were transformed (2.5 kV, 200 Ω, 25 μF) with 10 ng μL⁻¹ of the respective plasmid and recovered for 1 hour in 500 μL LB medium [10 g L⁻¹ tryptone (Oxoid), 5 g L⁻¹ yeast extract (BD), 10 g L⁻¹ NaCl (Acros)] at 37° C. Then, the recovered culture was serially diluted and drop plated on selective (50 μg mL⁻¹ kanamycin) LB agar plates in the presence or absence of 2 mM theophylline. The agar plates were incubated at 30° C. for 24 hours and the CFUs were counted.

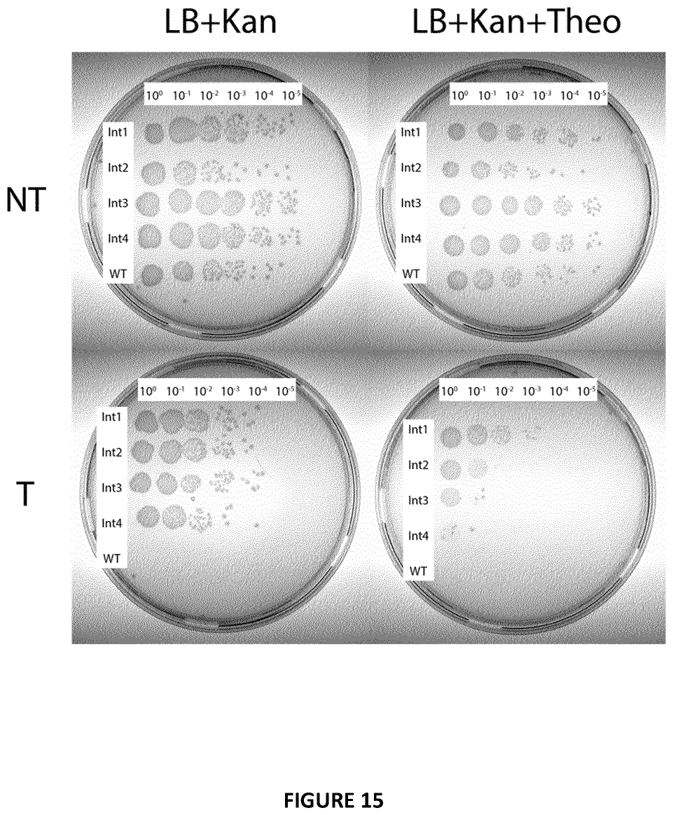

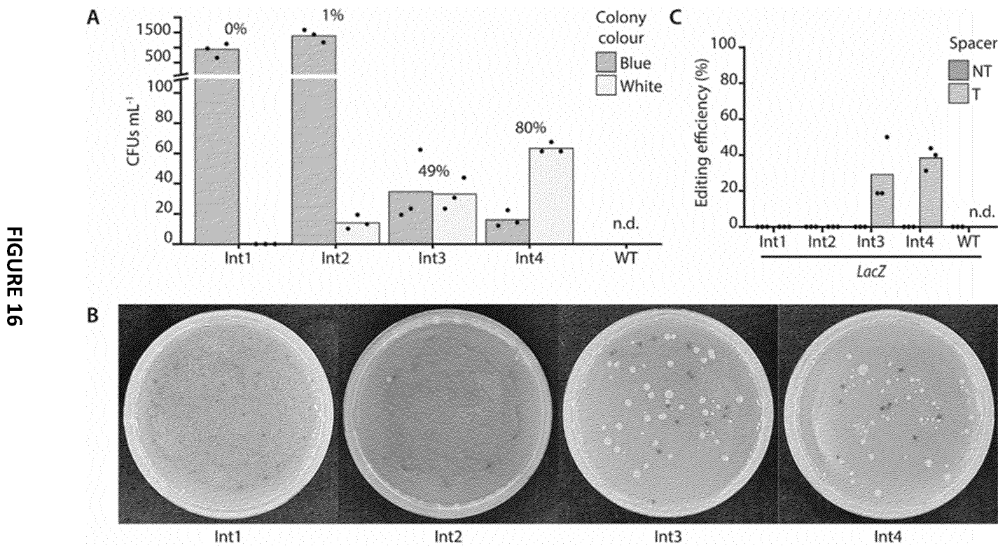

FIG. 15 shows the efficacy of the tagged-introns to control the activity of FnCas12a in E. coli MG1655. E. coli MG1655 was transformed with a series of plasmids (listed in Table 4) and serially diluted and drop plated on selective (kanamycin) LB media with or without 2 mM theophylline. All the transformants bearing an NT crRNA did not show reduction in CFUs on selective media with or without theophylline. In contrast, transformants bearing the T crRNA showed an obvious reduction in CFUs when plated on selection medium with theophylline. The effect of the CFU reduction was more apparent for the Tag4-intron where colonies could only be observed at the 10° dilution, followed by Tag3-intron (10⁻¹) then Tag2-intron (10⁻²) and finally Tag1-intron (10⁻²). The intron-less control transformation showed complete elimination of CFUs in both induced and non-induced selective media. Collectively, these results show the tight control of the FnCas12a gene and its inducibility using theophylline for the splicing of the T4 td intron variants.

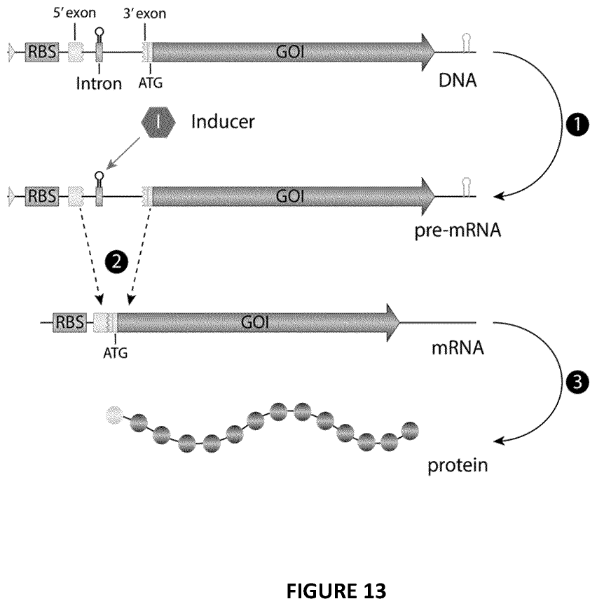

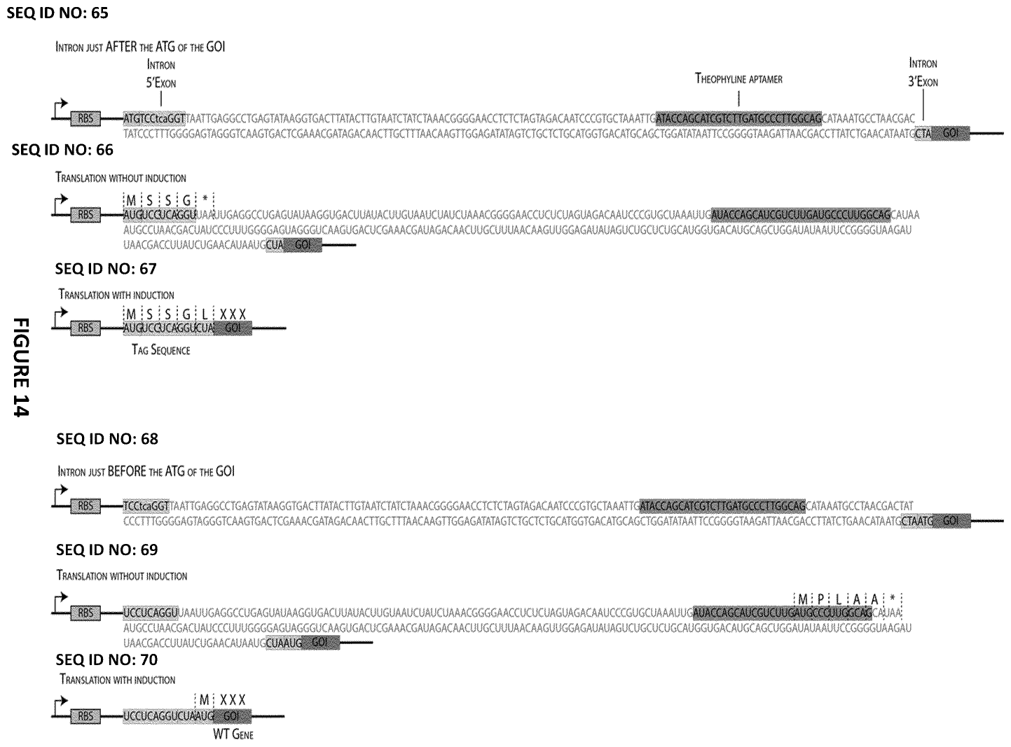

FIG. 13 shows an alternative method of steps in DNA to RNA to protein transcription with the intron positioned directly before the ATG start codon of the gene of interest. This setting prevents the ribosome to find an appropriate start codon in close proximity and therefore protein translation is inhibited. However, upon induction/splicing of the intron, the GOI comes closer to the RBS and therefore can be translated successfully and without a tag sequence attached to it.

FIG. 14 provides more detailed sequence based information for examples of methods where the self-splicing intron is placed just after the ATG of the GOI, or just before the ATG of the GOI.

### Example 6: Efficient Homologous Recombination in E. coli MG1655 Using T4 td Intron Variants

For efficient genome editing in bacteria, HR should precede CRISPR-Cas counterselection. To assess whether tight control over CRISPR-Cas targeting could bolster the efficiency of CRISPR-Cas mediated genome editing by allowing more time for HR to occur, we used SIBR-Cas and targeted the LacZ gene of E. coli MG1655 for knock-out through HR and CRISPR-Cas counterselection using a blue/white screening colony assay. To facilitate HR, we added 500 bp up- and down-stream homology arms to the plasmids expressing the four SIBR-Cas (Int1-4) and WT-FnCas12a variants that target the LacZ gene. After 1 hour recovery, we induced the expression of the SIBR-Cas variants to counterselect the WT from the mutant colonies.

The WT-FnCas12a variant targeting the LacZ gene produced no colonies, demonstrating the targeting efficiency of WT-FnCas12a but also the inefficient HR system of the WT E. coli MG1655 strain (FIG. 16A). In contrast, SIBR-Cas variants produced multiple colonies of which 80% of the total CFUs mL⁻¹ were white when Int4 was used, followed by Int3 (49%), Int2 (1%) and Int1 (0%) variants (FIGS. 16A and 16B). Similar to the previous results, the high editing efficiencies obtained with Int4 suggest that its high splicing efficiency translates into a stronger counter-selective pressure. No white colonies were observed for the non-targeting controls, demonstrating that the efficiency of editing without CRISPR-Cas counterselection is negligible.

Since disruption of LacZ can also be achieved through non-HR mediated approaches (spontaneous mutations or occasional error-prone DNA repair following DNA cleavage by Cas12a), not all gene deletions can be screened phenotypically. Therefore, we repeated our experiment, but X-gal was omitted from the medium to eliminate the possibility of false-positives. Randomly selected colonies that were obtained were screened by PCR for LacZ deletion showing a 0%, 0%, 29% and 38% editing efficiency for Int1, Int2, Int3 and Int4 SIBR-Cas variants, respectively (FIG. 24C). The WT-FnCas12a variant targeting LacZ did not yield any colonies and all the colonies obtained from the NT controls had the intact, wild-type LacZ locus. The observed decrease in editing efficiency (compared to the blue/white screening) might be attributed to spontaneous LacZ mutations that escape CRISPR-Cas counterselection. Nevertheless, a high editing efficiency (38%) was observed when SIBR-Cas Int4 was used without the use of recombinases or any other complex systems.

### Example 7: Tight and Inducible Expression of FnCas12a in Pseudomonas putida Using Tagged-T4 td Intron Variants

Following successful demonstration of inducible expression of FnCas12a in E. coli, the system was transferred to Pseudomonas putida, an organism with very low HR efficiencies. Plasmids bearing the four T4 td intron-FnCas12a or the intron-less FnCas12a and an EndA T or an NT crRNA were transformed to P. putida and the targeting efficiency was assessed by comparing the CFUs μg⁻¹ in the presence or absence of the theophylline inducer. All the plasmids used for this experiment are listed in Table 5.

TABLE 5
Plasmids used for targeting in P. putida.

| Plasmid name | Description and relevant characteristics |
|---|---|
| pSIBR EcoPpu NT tag 1 | KanR, FnCas12a with Tag1-intron, NT crRNA |
| pSIBR EcoPpu NT tag 2 | KanR, FnCas12a with Tag2-intron, NT crRNA |
| pSIBR EcoPpu NT tag 3 | KanR, FnCas12a with Tag3-intron, NT crRNA |
| pSIBR EcoPpu NT tag 4 | KanR, FnCas12a with Tag4-intron, NT crRNA |
| pSIBR EcoPpu NT no intron | KanR, WT FnCas12a, NT crRNA |
| pSIBR EcoPpu T EndA tag 1 | KanR, FnCas12a with Tag1-intron, EndA T crRNA |
| pSIBR EcoPpu T EndA tag 2 | KanR, FnCas12a with Tag2-intron, EndA T crRNA |
| pSIBR EcoPpu T EndA tag 3 | KanR, FnCas12a with Tag3-intron, EndA T crRNA |
| pSIBR EcoPpu T EndA tag 4 | KanR, FnCas12a with Tag4-intron, EndA T crRNA |
| pSIBR EcoPpu T EndA no intron | KanR, WT FnCas12a, EndA T crRNA |

In more detail, electrocompetent P. putida cells were transformed (2.5 kV, 200 Ω, 25 μF) with 200 ng plasmid and recovered in 1 ml LB for 2 hours at 30° C. Then, the culture was serially diluted and drop plated on selective (50 μg mL⁻¹ kanamycin) LB agar plates in the presence or absence of 2 mM theophylline. The agar plates were incubated at 30° C. for 24 hours and the CFUs were counted.

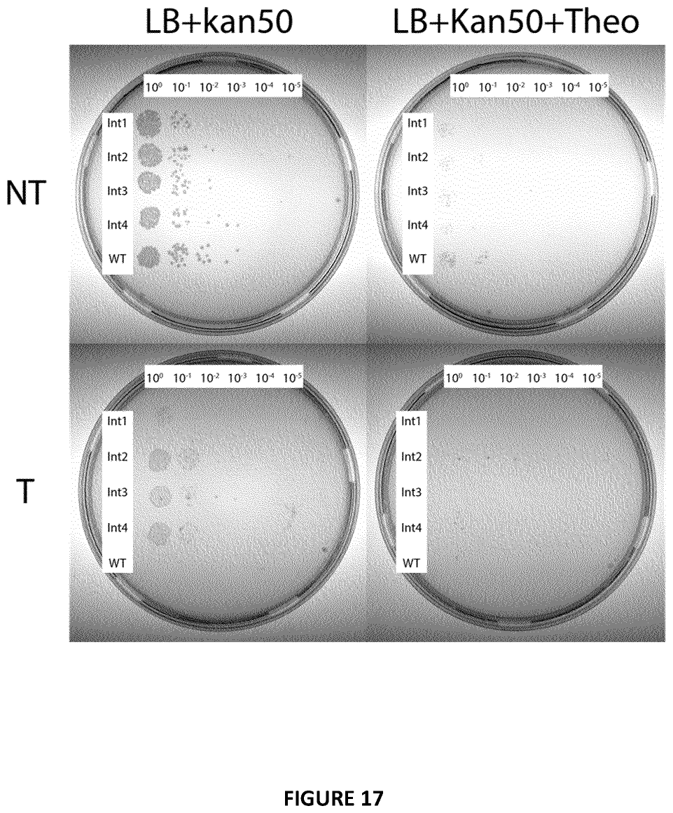

FIG. 17 shows the efficacy of the tagged-introns to control the activity of FnCas12a in P. putida. P. putida was transformed with a series of plasmids (listed in Table 6) and serially diluted and drop plated on selective (kanamycin) LB media with or without 2 mM theophylline. Similarly to the results in E. coli (Example 5 above), only the transformants bearing the T crRNA and plated on selective media with theophylline showed reduced CFUs. However, for P. putida, the effect of induction was more apparent than E. coli as no CFUs could be observed for transformants bearing the T crRNA and plated on selective media with theophylline. The intron-less controls did not have any CFUs in media with or without theophylline, showing the constitutive expression and hence targeting on the FnCas12a protein. Collectively, the Tagged-intron constructs work efficiently in P. putida and can be used for genome engineering.

### Example 8: Efficient Homologous Recombination in P. putida Using Tagged-T4 td Intron Variants

Further genome editing experiments were conducted to knock-out the FlgM gene of P. putida. A repair template (1125 bp) was included on the plasmids bearing approximately 500 bp homologous sides upstream and downstream of the FlgM gene. The repair template was introduced to either of the four tagged-intron-FnCas12a variants along with the T crRNA for counterselection or the NT crRNA as a control. A list of the plasmids is given in table 6. Plasmids were transformed to P. putida through electroporation and the transformed cells were recovered in LB medium for 2 hours before plating on LB agar plates containing 50 μg ml⁻¹ kanamycin and 2 mM theophylline. Plates were incubated at 30° C. overnight and formed colonies were screened through colony PCR for the knock-out of the FlgM gene. FIG. 18 shows the knock-out efficiency of the FlgM gene in P. putida. Tag1 to Tag4 represent the four different Tagged-intron variant plasmids which contain the T (targeting) or the NT (non-targeting) crRNA. Transformants bearing the Tag4 intron-FnCas12a variant showed 70% editing efficiency whereas transformants bearing the Tag3, 2 or Tag1 intron-FnCas12a variant showed 36.6%, 39.3% and 0% editing efficiency, respectively. On the contrary, transformants bearing plasmids with an NT crRNA were all WT.

TABLE 6
Plasmids used for homologous recombination in P. putida.

| Plasmid name | Description and relevant characteristics |
|---|---|
| pSIBR EcoPpu NT FlgM HA tag 1 | KanR, FnCas12a with Tag1-intron, NT crRNA, |
| | Homologous arms for FlgM |
| pSIBR EcoPpu NT FlgM HA tag 2 | KanR, FnCas12a with Tag2-intron, NT crRNA, |
| | Homologous arms for FlgM |
| pSIBR EcoPpu NT FlgM HA tag 3 | KanR, FnCas12a with Tag3-intron, NT crRNA, |
| | Homologous arms for FlgM |
| pSIBR EcoPpu NT FlgM HA tag 4 | KanR, FnCas12a with Tag4-intron, NT crRNA, |
| | Homologous arms for FlgM |
| pSIBR EcoPpu T FlgM HA tag 1 | KanR, FnCas12a with Tag1-intron, EndA T |
| | crRNA, Homologous arms for FlgM |
| pSIBR EcoPpu T FlgM HA tag 2 | KanR, FnCas12a with Tag2-intron, EndA T |
| | crRNA, Homologous arms for FlgM |
| pSIBR EcoPpu T FlgM HA tag 3 | KanR, FnCas12a with Tag3-intron, EndA T |
| | crRNA, Homologous arms for FlgM |
| pSIBR EcoPpu T FlgM HA tag 4 | KanR, FnCas12a with Tag4-intron, EndA T |
| | crRNA, Homologous arms for FlgM |

### Example 9: Using a Theophylline Induced Self-Splicing Intron to Control the Expression of Cas12a and Knock Out the SprF Essential Gene in the Non-Model Organism Flavobacterium IR1

Flavobacterium IR1 is a non-model organism known for its iridescent colour (see Johansen, V., et al., (2018) “Genetic manipulation of structural colour in bacterial colonies” Proceedings of the National Academy of Sciences 115 (11): 2652-2657; and Schertel, L., G. T. et al., (2020) “Complex photonic response reveals three-dimensional self-organization of structural coloured bacterial colonies” Journal of the Royal Society Interface 17 (166): 20200196). The lack of genomic tools and the low HR efficiency of IR1 are currently the main bottlenecks limiting the fundamental characterization and commercial exploitation of this phenomenon (i.e. development of new paints). As IR1 is a recently discovered non-model organism, inducible promoters are not characterized. Therefore, the control of CRISPR-Cas cannot succeed without a promoter-independent regulatory system such as is disclosed herein.

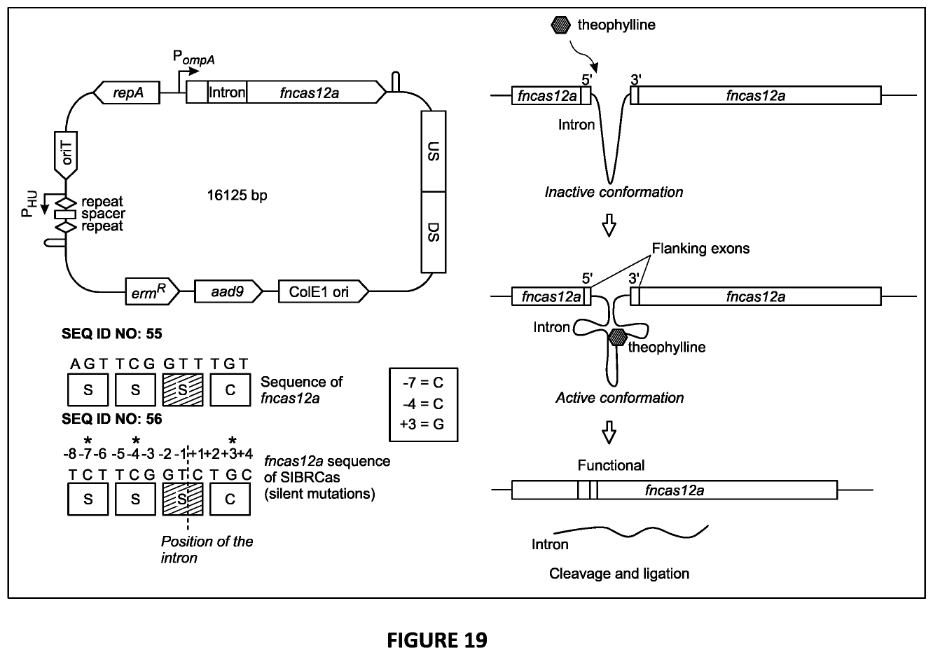

To establish controllable genetic engineering tools for IR1, plasmids were constructed by inserting the 300 bp self-splicing aptazyme intron of Thompson et al., (2002) Supra into the fncas12a gene to provide a module, and subsequently inserting this module into two editing plasmids yielding pSIBRFnCas12a_sprF_HR_NT (no-target spacer) and pSIBRFnCas12a_sprF_HR_S3 (spacer targeting sprF gene). For this, the theophylline T4 td intron was introduced in the ORF of FnCas12a. The insertion position was generated by using the algorithm of Example 11. The insertion position is illustrated in FIG. 18. As shown in FIG. 19, mutations were introduced into the FnCas12a sequence flanking the 5′ and 3′ of the intron in order to maintain the self-splicing activity of the ribozyme. As shown in FIG. 19, there is a map of the plasmid containing FnCas12a gene disrupted with the T4 td intron. Below the plasmid map is shown the 5′ and 3′ exon without (upper) and with (lower) silent mutations. The silent mutations were made to suit the −7, −4, and +296 positions of the exon. On the right of FIG. 19 is shown the mRNA of FnCas12a containing the T4 td intron. In the presence of theophylline, the intron forms a secondary structure which induces self-splicing and results in a correct mRNA which expresses functional FnCas12a.

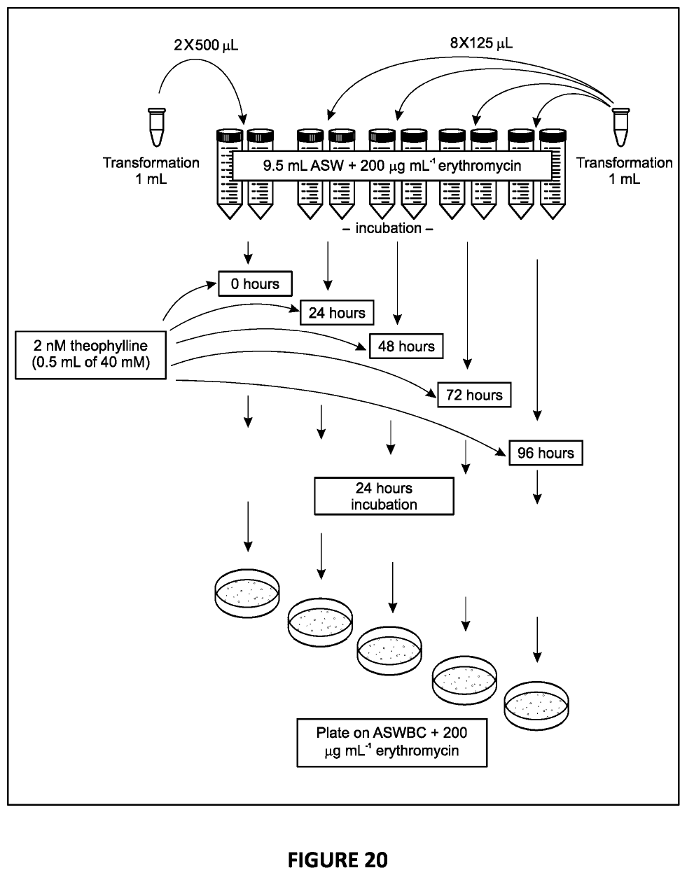

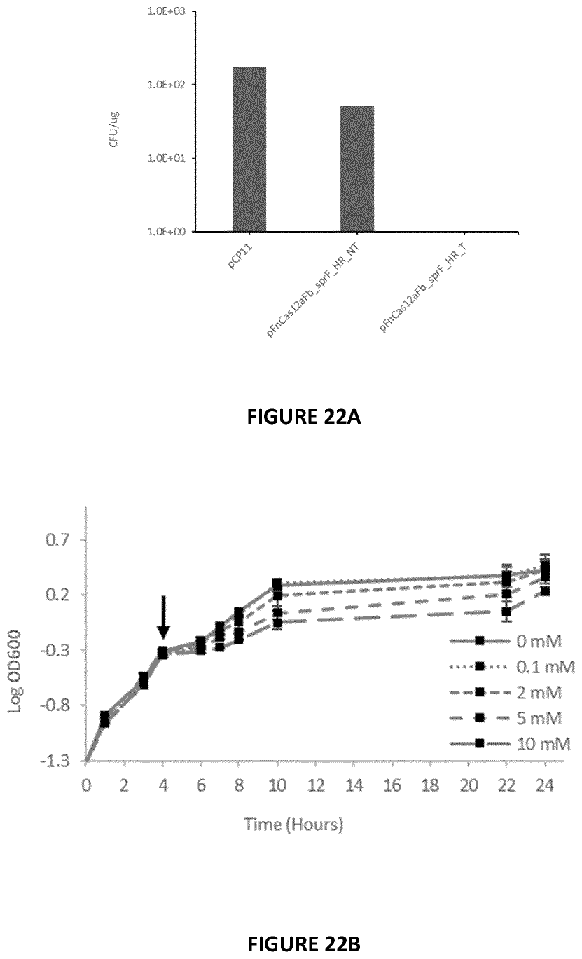

The constructed plasmids were then transformed into IR1 and cultured following the experimental design shown in FIG. 20. An adopted method can be used as shown in FIG. 22. This setting was applied for both pRiboFnCas12a_sprF_HR_NT and pRiboFnCas12a_sprF_HR_S3. Each different treatment (incubation time variation) was done in duplicate.

Prior to theophylline induction, the liquid cultures of IR1 transformed with pSIBRFnCas12a_sprF_HR_S3 and incubated for 0, 24, and 48 h showed no obvious growth (data not shown). Correspondingly, there was no colony obtained after plating these cultures following theophylline induction in the liquid culture. In contrast, IR1 transformed with the non-targeting plasmid pSIBRFnCas12aFb_sprF_HR_NT showed growth following 24 and 48 h incubation prior and after the induction with theophylline.

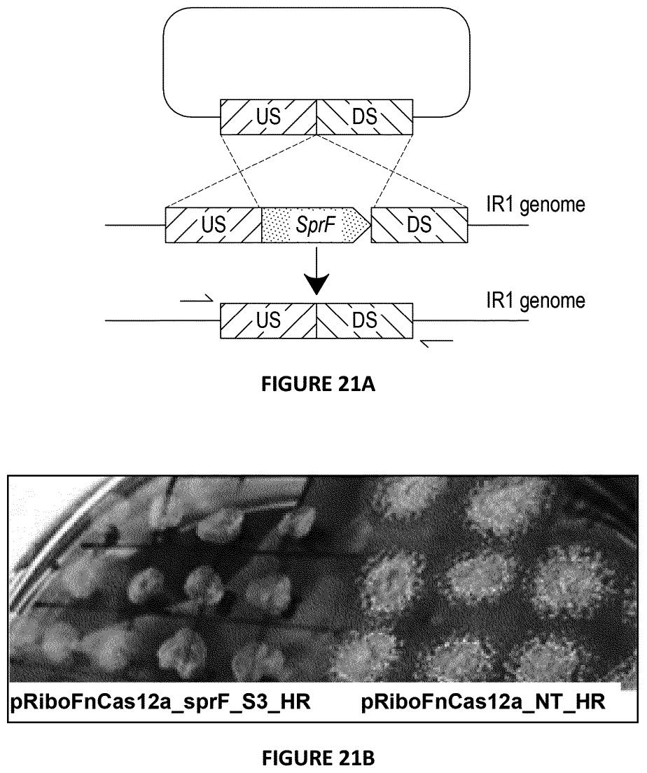

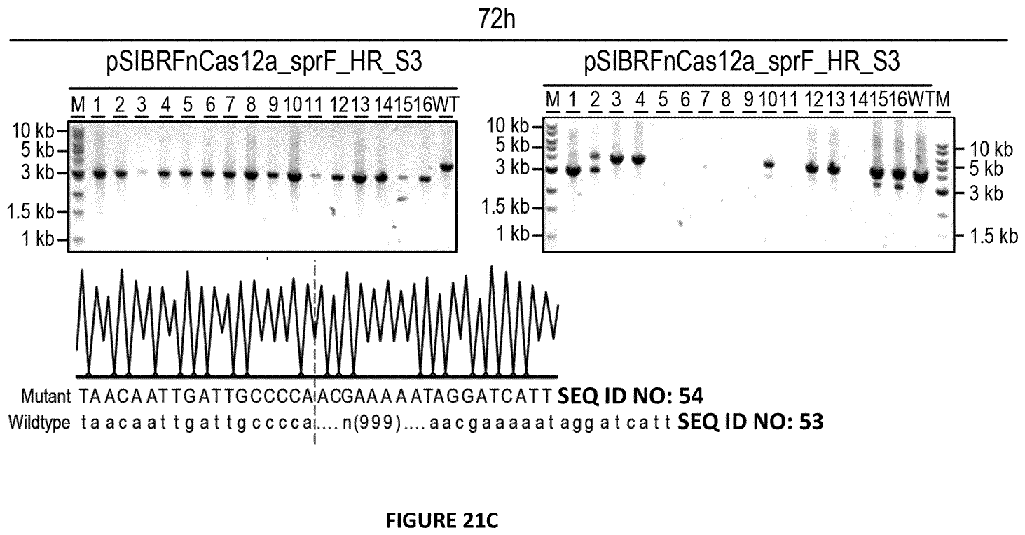

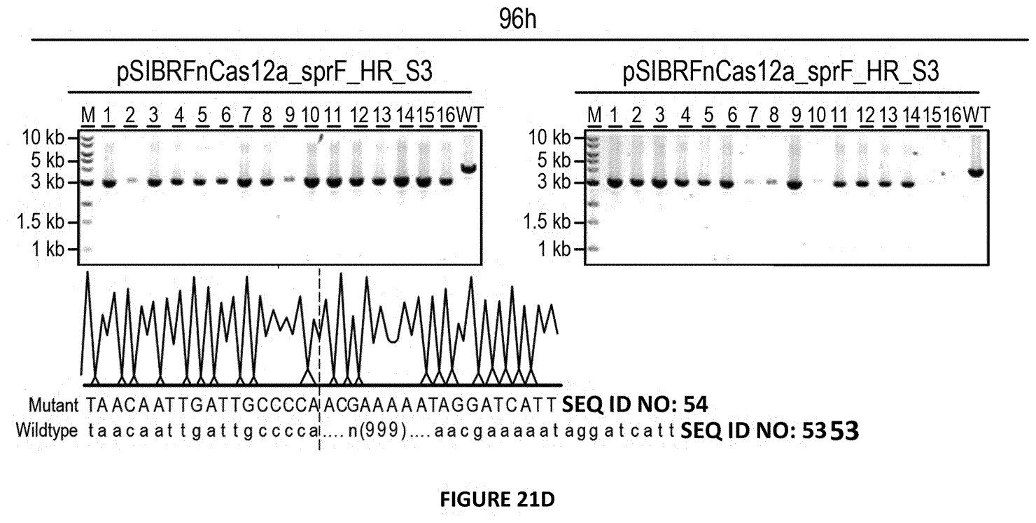

FIG. 21A is a schematic representation of SprF gene deletion achieved in the genome of IR1. FIG. 21B shows Flavobacterium sp. IR1 cells transformed with pSIBRFnCas12a_sprF_S3_HR (left) and pSIBRFnCas12a_NT_HR (right) and grown in ASWBC agar. The loss of structural colour in the colonies is suggested as the result of SprF gene deletion. FIG. 21C shows Agarose gel electrophoresis showing the results from colony PCR on the colonies (1-16) of Flavobacterium sp. IR1 cells transformed with pSIBRFnCas12a_sprF_S3_HR after 72 h incubation in duplicates. The culture 1 (left) resulted in all tested colonies being knocked out (100% editing efficiency). Meanwhile, in culture 2 (right) 1 colonies was knockout mutant and 6 colonies were mixed knockout and wildtype. FIG. 21D shows Agarose gel electrophoresis showing the results from colony PCR on the colonies (1-16) of Flavobacterium sp. IR1 cells transformed with pSIBRFnCas12a_sprF_S3_HR after 96 h incubation in duplicates. Both cultures showed 16 out of 16 colonies were knockout SprF mutants (100%). The colonies were obtained after plating the transformants grown for 72 and 96 h in liquid culture and followed by induction of 2 mM of theophylline in liquid with additional 24 h incubation. A band at 3038 bp indicates the presence of the correct ΔSprF mutant corresponding to the deleted 999 bp SprF gene. The last lane is the negative (wild-type) control that corresponds to an 4037 bp long DNA fragment.

Interestingly, after 72 h and 96 h incubation, cultures transformed with pSIBRFnCas12a_sprF_HR_S3 started to show some growth (data not shown). Likewise, colonies were also obtained when plating these cultures after theophylline induction. FIG. 21B shows structure and colour of the colonies transformed with pSIBRFnCas12a_sprF_HR_S3 with 72 h and 96 h was drastically reduced compared to the colonies transformed with pSIBRFnCas12aFb_sprF_HR_NT. This phenotype was the expected phenotype for disrupting/knocking-out the target gene, sprF, as previously reported (Johansen, V., et al., (2018) Supra. As shown in FIG. 21C, PCR screening of these colonies showed that after 72 h incubation, correctly edited mutants were obtained with editing efficiency from 43% up to 100%. As shown in FIG. 21D, 96 h incubation editing efficiency of the Cas system was maintained at 100% in two replicates. Two colonies that appeared to be knock-outs, were further confirmed by Sanger sequencing as shown in both FIGS. 21C and 21D.

### Example 10: Highly Efficient Homologous Recombination in the Non-Model Organism Flavobacterium IR1

To demonstrate the inefficient HR mechanism of IR1, the organism was transformed using electroporation with a plasmid expressing an intron-less (WT) FnCas12a under a constitutive promoter (OmpA-P), and a T crRNA targeting the SprF gene under the constitutive promoter HU-P. A repair template (2963 bp) for knocking out the SprF gene through HR was also included on the plasmid resulting in the final plasmid pFnCas12aFb_sprF_HR_T. As control, the crRNA was replaced with an NT crRNA resulting in pFnCas12aFb_sprF_HR_NT. Also, the pCP11 empty vector was used as an indicator for transformation efficiency.

IR1 electrocompetent cells were prepared as follows: IR1 was grown overnight in 10 mL of ASW at 25° C., 200 rpm. The overnight culture was used to inoculate 2×100 mL ASW broth in 500 ml baffled flask and grown until it reached an OD600 of 0.3. Thereafter, the cells were harvested by centrifugation at 4000 rpm for 10 minutes, 4° C. The cells were washed two times with 1×volume of washing buffer (10 mM MgCl₂ and 5 mM CaCl₂) at 4° C. and washed once with 10% (v/v) glycerol (Gilchrist and Smit, (1991) “Transformation of freshwater and marine caulobacters by electroporation” Journal of Bacteriology 173.2: 921-925). The pellet was suspended using 10% glycerol to 1/100 of the initial volume. Cells were divided into aliquots of 100 μL in 1.5 mL Eppendorf tubes and stored at −80° C. until use.

IR1 electrocompetent cells were transformed with 1 μg μl⁻¹ plasmid in 1-mm cuvette using the following settings: 1.5 kV, 200 Ω, 25 μF. 900 μL of ASW medium [5 g L⁻¹ peptone (Sigma #70173), 1 g L⁻¹ yeast extract (BD), 10 g L⁻¹ sea salt (Sel marin)] was added immediately and the cells were incubated at 25° C. for 4 hours for recovery. The cells were plated on ASWBC agar [ASW medium, 15 g L⁻¹ agar (Oxoid), 100 mg L⁻¹ nigrosine (Aldrich #198285), and 5 g L⁻¹ Kappa Carrageenan (Special Ingredients)] supplemented with 200 μg mL⁻¹ erythromycin and incubated at 25° C. for 2 to 3 days.

FIG. 22 shows the transformation efficiency and theophylline toxicity for Flavobacterium IR1. FIG. 22A shows the transformation efficiency using the empty vector pCP11, the control pFnCas12aFb-NT and the pFnCas12aFb-T (targeting the SprF gene) plasmids. Targeting the SprF gene did not yield any viable colonies even after recovery for 4 hours. NT control and pCP11 empty vector result 52 and 176 colonies respectively. So, as shown in FIG. 22A, and as predicted, no colonies could be obtained when IR1 was transformed with pFnCas12aFb_sprF_HR_S3. But, an average of 52 CFUs/μg was obtained when using the pFnCas12aFb_sprF_HR_NT control and 176 CFUs/μg when using the pCP11 empty vector.

Clearly, the constitutive expression of the WT FnCas12a and the T crRNA along with the inefficient HR machinery of IR1 resulted in the targeting of the genome of IR1 causing cell death. To overcome this limitation, it is suggested to use the four T4 td intron-FnCas12a variants (as developed for E. coli and P. putida) and this would result in the tight and controlled expression of FnCas12a in order to allow HR to precede counterselection.

Because theophylline uptake from IR1 appears to be a prerequisite, a toxicity assay was carried out on the growth of IR with varying theophylline concentrations (0, 0.1, 2, 5 and mM) grown for 24 hours at 25° C. FIG. 22B shows the theophylline toxicity at 0, 0.1, 2, and 10 mM of theophylline concentration in the growth medium of IR1. The arrow indicates the time when theophylline was added to the medium. Theophylline concentration of up to 2 mM did not affect significantly the growth of IR1. However, 5 and 10 mM of Theophylline decreased the growth of IR1 indicating toxicity but also uptake of theophylline. Therefore, 2 mM of theophylline was used for inducing the intron splicing in following experiments.

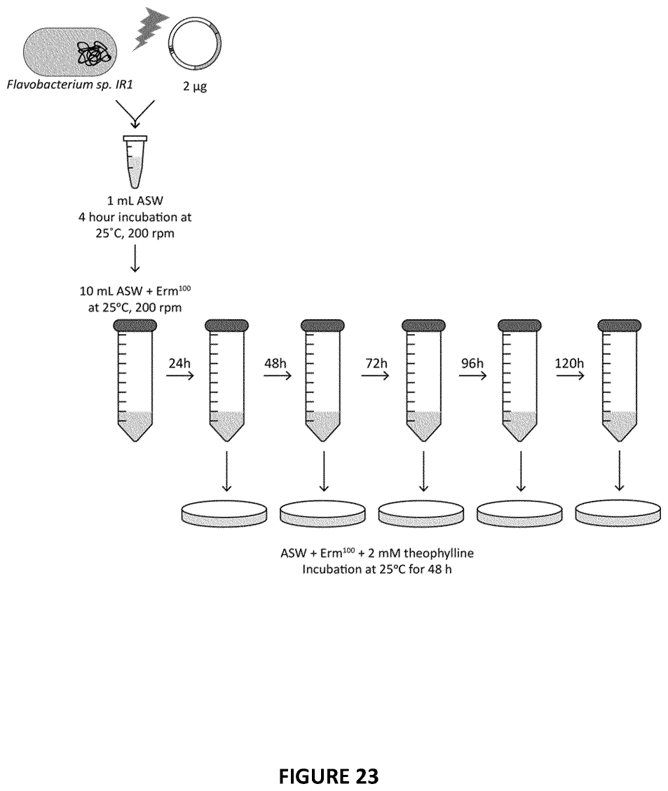

To achieve efficient HR in IR1, the WT FnCas12a in pFnCas12aFb_sprF_HR_S3 was replaced with the four T4 td intron-FnCas12a variants developed previously for E. coli and P. putida resulting in the plasmids listed in table 7. As a control, the WT FnCas12a of pFnCas12aFb_sprF_HR_NT was replaced with the four T4 td intron-FnCas12a variants (Table 7). In addition, a better method was developed in order to increase the obtained colonies as this will increase the chances of obtaining knock-outs. FIG. 23 shows schematically the procedure for obtaining knock-outs in Flavobacterium IR1. 2 μg of plasmid were transformed to electrocompetent Flavobacterium IR1 and recovered in 1 ml ASW medium for 4 hours at 25° C. shaking at 200 rpm. The recovered culture was then inoculated in 10 ml ASW selective medium (erythromycin) and grown at 25° C. shaking at 200 rpm for a total of 120 hours. Every 24 hours, a 1 ml sample was recovered, centrifuged at 4700 rpm for 5 mins and the concentrated cell pellet was plated on selective ASW agar plates containing 2 mM theophylline and incubated for 48 h at 25° C. The formed colonies were then screened through colony PCR for the presence or the absence of the target gene. Obtained colonies were screened by colony PCR for knock-outs.

FIG. 14 shows the results of the editing efficiencies of the SprF gene of Flavobacterium IR1 using the four different tagged-intron variants. The editing efficiencies are indicated in the bar graphs. Colonies (if any) were analysed for knock-out of the SprF gene through colony PCR every 24 hours. The table below the bar graph indicates the editing efficiency per biological replicate. Colony PCR showed knock-out efficiencies of up to 100% when Tag 1-, 3- and 4-Intron variants were used. NT controls showed only WT colonies confirming the efficiency of our tool in counterselecting the correct knock-outs.

TABLE 7
plasmids used for homologous recombination in Flavobacterium IR1.

| Plasmid name | Description and relevant characteristics |
|---|---|
| pSIBR Flavo NT SprF HA tag 1 | SpecR for E. coli, ErmR for Flavo, FnCas12a with Tag1- |
| | intron, NT crRNA, Homologous arms for FlgM |
| pSIBR Flavo NT SprF HA tag 2 | SpecR for E. coli, ErmR for Flavo, FnCas12a with Tag2- |
| | intron, NT crRNA, Homologous arms for FlgM |
| pSIBR Flavo NT SprF HA tag 3 | SpecR for E. coli, ErmR for Flavo, FnCas12a with Tag3- |
| | intron, NT crRNA, Homologous arms for FlgM |
| pSIBR Flavo NT SprF HA tag 4 | SpecR for E. coli, ErmR for Flavo, FnCas12a with Tag4- |
| | intron, NT crRNA, Homologous arms for FlgM |
| pSIBR Flavo T SprF HA tag 1 | SpecR for E. coli, ErmR for Flavo, FnCas12a with Tag1- |
| | intron, T crRNA, Homologous arms for FlgM |
| pSIBR Flavo T SprF HA tag 2 | SpecR for E. coli, ErmR for Flavo, FnCas12a with Tag2- |
| | intron, T crRNA, Homologous arms for FlgM |
| pSIBR Flavo T SprF HA tag 3 | SpecR for E. coli, ErmR for Flavo, FnCas12a with Tag3- |
| | intron, T crRNA, Homologous arms for FlgM |
| pSIBR Flavo T SprF HA tag 4 | SpecR for E. coli, ErmR for Flavo, FnCas12a with Tag4- |
| | intron, T crRNA, Homologous arms for FlgM |

### Example 11: Python Script Used to Find Insertion Sites

By using the following script, the user can upload the sequence of the gene of interest and the script will return possible insertion sites for the T4 td intron. The insertion sites will require point mutations to be introduced when inserting the intron at the target site. Multiple sites are possible options but one at the beginning of the gene is recommended to eliminate potential function of partially produced proteins.

```
def findbindingtype(Q, S):
  a = M″
  if Q = = ″T″ and S = = ″A″: a = ″P″
  if Q = = ″T″ and S = = ″G″: a = ″W″
  if Q = = ″C″ and S = = ″G″: a = ″P″
  if Q = = ″A″ and S = = ″T″: a = ″P″
  if Q = = ″G″ and S = = ″C″: a = ″P″
  if Q = = ″G″ and S = = ″T″: a = ″W″
  if Q = = ″N″ or S = = ″N″: a = ″X″
  return a
def permutatelist(X):
  Y = [″″]
  for i in X:
    Z = Y
    Y = [ ]
    for j in i:
      for I in range(len(Z)):
        Y.append(Z[I]+j)
  return Y
def matchsense(q, s):
  a = 100.000
  if q = = s: a = 1.000
  if q = = ″R″ and (s = = ″A″ or s = = ″G″): a = 1.000
  if q = = ″Y″ and (s = = ″C″ or s = = ″T″): a = 1.000
  if q = = ″S″ and (s = = ″G″ or s = = ″C″): a = 1.000
  if q = = ″W″ and (s = = ″A″ or s = = ″T″): a = 1.000
  if q = = ″K″ and (s = = ″G″ or s = = ″T″): a = 1.000
  if q = = ″M″ and (s = = ″A″ or s = = ″C″): a = 1.000
  if q = = ″B″ and s = ″A″: a = 1.000
  if q = = ″D″ and s != ″C″: a = 1.000
  if q = = ″H″ and s != ″G″: a = 1.000
  if q = = ″V″ and s != ″T″: a = 1.000
  if q = = ″N″: a = 1.000
  return a
def RevComp(sequence):
  RC = ″″
  for i in sequence:
    j = ″″
    if i = = ″T″: j = ″A″
    if i = = ″C″: j = ″G″
    if i = = ″A″: j = ″T″
    if i = = ″G″: j = ″C″
    if i = = ″Y″: j = ″R″
    if i = = ″W″: j = ″W″
    if i = = ″K″: j = ″M″
    if i = = ″M″: j = ″K″
    if i = = ″S″: j = ″S″
    if i = = ″R″: j = ″Y″
    if i = = ″H″: j = ″G″
    if i = = ″B″: j = ″A″
    if i = = ″D″: j = ″C″
    if i = = ″V″: j = ″T″
    if i = = ″N″: j = ″N″
    RC = j + RC
return RC
#-------------- Input Subject -------------------------
SubDNA = [ ]
FN = raw_input(′DNA File name = ′)
with open(FN, ′r+′) as f:
  for r in f:
    for c in r:
      if c.upper( ) = = ″G″ or c.upper( ) = = ″A″ or c.upper( ) = = ″T″ or c.upper( ) = = ″C″:
        SubDNA.append(c.upper( ))
#-------------- Input Query ---------------------------
# −8 −7 −6 −5 −4 −3 −2 −1 1 2 3 4
QueDNA = [″N″ ,″T″ ,″G″ ,″A″ ,″G″ ,″T″, ″C″, ″C″ ,″G″ ,″G″ ,″A″ ,″G″ ,″T″] #Native
QueDNA = [″N″ ,″T″ ,″G″ ,″A″ ,″G″ ,″T″ ,″C″ ,″C″ ,″G″ ,″G″ ,″A″ ,″G″ ] #Simplified
#″Query Functions:
#(P)air, (W)obbly, (N)onbinding″)
#(F)ixed (P)air, (F)ixed (W)obbly, (F)ixed (N)onbinding
#-------------- Import Codon Table --------------------
AA = [ ]
Codon = [ ]
import csv
with open(′Codon.csv′) as csvfile:
  f = csv.reader(csvfile, delimiter=′,′, quotechar=′|′)
  for r in f:
    Codon.append(r[0])
    AA.append(r[1])
#-------------- Translate Subject ---------------------
SubPro = [ ]
for i in range(int(len(SubDNA)/3)):
  qcodon = SubDNA[3*i]+SubDNA[3*i+1]+SubDNA[3*i+2]
  for j in range(64):
    if Codon[j] = = qcodon:
      SubPro.append(AA[j])
#--------------- Define Peptide -----------------------
TBS_DNA = [ ]
TBS_Pos = [ ]
for s in range(len(SubPro)-int((len(QueDNA)+4)/3)−1): #Subject Protein Start
  Peptide = SubPro[s:s+int((len(QueDNA)+4)/3)]
#--------------- Reverse Translate --------------------
  mRNA = [ ]
  for i in Peptide:
    Cdn = [ ]
    for j in range(64):
      if AA[j] = = i:
        Cdn.append(Codon[j])
  mRNA.append(Cdn)
RevTrans = permutatelist(mRNA)
TBS_DNA = TBS_DNA + RevTrans
for k in RevTrans:
  TBS_Pos.append(s)
#--------------- Test binding type --------------------
print ″Binding Type analysis″
ScoreTable_BP = [ ]
ScoreTable_Score = [ ]
import csv
with open(′TdScoreTable.csv′) as csvfile:
  f = csv.reader(csvfile, delimiter=′,′, quotechar=′|′)
  for r in f:
    ScoreTable_BP.append(r[0])
    ScoreTable_Score.append(r[1])
DNA_List = [ ]
Pos_List = [ ]
Score_List = [ ]
Pep_List = [ ]
Frame_List = [ ]
Query = [″N″,″N″,″N″] + QueDNA + [″N″,″N″,″N″]
for i in range(len(TBS_DNA)): #Subject list
  Subject = TBS_DNA[i]
  for f in range(3): #Frame select
    BP = ″″
    for j in range(len(QueDNA)):
      BP = BP + findbindingtype(QueDNA[j],Subject[j+f])
    Score = 0
    for k in range(len(ScoreTable_BP)):
      if BP = = ScoreTable_BP[k]:
      Score = ScoreTable_Score[k]
      DNA_List.append(TBS_DNA[i])
      Pos_List.append(TBS_Pos[i]+1)
      Score_List.append(Score)
      Frame_List.append(f+1)
      Peptide = ″″
      for I in SubPro[TBS_Pos[i]: TBS_Pos[i]+int((len(QueDNA)+4)/3)]:
        Peptide = Peptide + I
      Pep_List.append(Peptide)
#--------------- select 5 best ------------------------
Result = [ ]
for row in range(len(Pos_List)) :
  Result.append ([Pos_List[row], DNA_List[row], Pep_List[row], Score_List[row],
Frame_List[row]])
Result2 = sorted(sorted(Result, key=lambda A: A[3], reverse=True), key=lambda A:
A[0])
A = Results2[0][0]
count = 0
Result3 = [ ]
for r in range(len(Result2)):
  if Result2[r][0] = = A and count <= 5:
    Result3.append(Result2[r])
    count = count + 1
  if Result2[r][0] != A:
    A = Result2 [r][0]
    count = 1
print Result3
#--------------- Export to CSV ------------------------
print ″Writing intron sites to file″
import csv
Writer = csv.writer(open(FN+″.csv″, ′wb′), delimiter=′,′)
Writer.writerow([″Position″, ″DNA″, ″Protein″, ″Score″, ″Frame″])
with open(FN+″.csv″, ′ab′) as F:
  for row in range(len(Result3)):
    Writer = csv.writer(F, delimiter=′,′)
    Writer.writerow(Result3[row])
#------------------Find the Restriction Enzymes-------------------------
#------------------Import RE Sequences------------------
PreREName = [ ]
PreRESeq = []
import csv
with open(′NEB RE.csv′) as csvfile:
  f = csv.reader(csvfile, delimiter=′,′, quotechar=′|′)
  for r in f:
    PreREName.append(r[0])
    PreRESeq.append(r[1])
#------------------Find RE in DNA Sequence--------------------------
print(″Analysing current Restriction endonulease sites″)
REName = [ ]
RESeq = [ ]
REPresent = [ ]
PreREPresent = [ ]
for e in range(len(PreRESeq)):
  QueDNA = [ ]
  s = 0
for c in PreRESeq[e]:
  QueDNA.append(c)
for i in range(len(SubDNA)-len(QueDNA)+1):
  score = 0
  for j in range(len(QueDNA)):
    score = score + matchsense(QueDNA[j],SubDNA[i+j])
  if score = = len(QueDNA):
    if s = = 0:
      Result = str(int((i+3)/3))
    if s > 0 and s <= 3:
      Result = Result + ″, ″ + str(int((i+3)/3))
    s = s + 1
if s > 0:
  PreREPresent.append(Result)
if s = = 0:
  PreREPresent.append(″N/A″)
if s < = 3:
  REPresent.append(PreREPresent[e])
  REName.append(PreREName[e])
  RESeq.append(PreRESeq[e])
#------------------Find RE is protein Sequence---------------------------
ResultName = [ ]
ResultPos = [ ]
ResultDNA = [ ]
ResultProtein = [ ]
ResultFrame = [ ]
ResultOtherRE = [ ]
import csv
Writer = csv.writer(open(FN+″-RE.csv″, ′wb′), delimiter=′,′)
Writer.writerow([″Enzyme″, ″Position″, ″DNA Seq″, ″AA Seq″, ″Frame″, ″RE sites
present″])
print(″Analysing potential Restriction endonulease sites″)
for e in range(len(RESeq)):
  QueDNA = [″N″,″N″,″N″]
  for c in RESeq[e]:
    QueDNA.append(c)
  QueDNA = QueDNA+[″N″]+[″N″]+[″N″]+[″N″]
  for f in range(3):
    for i in range(len(SubPro)-int((len(QueDNA)-3)/3)+1): #Subject amino acid start
      currentDNA = ″″
      currentProtein = ″″
      currentScore = 0
      m = 0 #Maxscore counter
      for j in range(int((len(QueDNA)−3)/3)): #Subject amino acid
        SubAA = SubPro[i+j]
        maxscore = 0
        for k in range(64): #64 codons/AA
          if SubAA = = AA[k]:
            score = 0
            QueCodon = Codon[k]
          for I in range(3):
            score = score + matchsense(QueDNA[3*j+3+I-f],QueCodon[I])
          if score<100 and score>maxscore:
            maxscore = score
            maxcodon = k
      if maxscore>0:
        m = m + 1
        currentDNA = currentDNA + Codon[maxcodon]
        currentProtein = currentProtein + AA[maxcodon]
        currentScore = currentScore + maxscore
    if m = = int((len(QueDNA)−3)/3):
      ResultName.append(REName[e])
      ResultPos.append(i+1)
      ResultDNA.append(currentDNA)
      ResultProtein.append(currentProtein)
      ResultFrame.append(f+1)
      ResultOtherRE.append(REPresent[e])
print(″Writing RE sites to file″)
import csv
with open(FN+″-RE.csv″, ′ab′) as F:
  for i in range(len(ResultName)):
    Writer = csv.writer(F, delimiter=′,′)
    Writer.writerow([ResultName[i], ResultPos[i], ResultDNA[i], ResultProtein[i],
ResultFrame[i], ResultOtherRE[i]])
print ″Done″
```

### Example 12: Using the SIBR T4 td Intron for Inducible Gene Expression into the Eukaryotic Model Organism Bakers Yeast (Saccharomyces cerevisiae)

To show the functionality and applicability of SIBR into eukaryotic systems, we transferred SIBR into the eukaryotic model organism Baker's yeast (Saccharomyces cerevisiae) and controlled the expression of the FnCas12a protein.

To control the activity of FnCas12a, we sought to disrupt its activity by disrupting the encoded protein through the placement of SIBR. To this end, by using the acquired knowledge from Example 1, 2 and 3 and the generated script from Example 4 and/or 11, we introduced SIBR before the RuvC I domain at amino acid position 859 (FIG. 25). Following the script from Example 4 and/or 11, the 5′ exonic sequence of the intron was 5′-AAAGAGTCGGT-3′ (SEQ ID NO: 76) in order not to disturb the amino acid sequence of FnCas12a upon excision of the intron (FIG. 25). The 3′ exonic sequence of the intron was 5′-CTT-3′ (SEQ ID NO: 77) to maintain the amino acid sequence of FnCas12a upon excision of the intron (FIG. 25). To this end, two plasmids were constructed which contained the modified intron (either with or without the theophylline aptamer) at amino acid position 859 of FnCas12a. The FnCas12a+intron gene constructs were expressed by the constitutive TEF1 promoter. Those plasmids were named PL-319 (FnCas12a+intron without aptamer) and PL-320 (FnCas12a+intron with aptamer). As a positive control for targeting, another plasmid was used (pUDE731; Addgene plasmid #103008) where the WT FnCas12a (no intron in the sequence of FnCas12a) was constitutively expressed by the TEF1 promoter.

PL-319, PL-320 or pUDE731 were co-transformed in the yeast S. cerevisiae with either a plasmid containing a non-targeting spacer (PL-207) or a plasmid containing a targeting spacer (PL-074). The targeting spacer was targeting the ADE2 gene. To transform S. cerevisiae, the LiAc/SS carrier DNA/PEG method by Gietz and Schiestl (Gietz, R. D. and Schiestl, R. H., 2007. High-efficiency yeast transformation using the LiAc/SS carrier DNA/PEG method. Nature protocols, 2(1), pp. 31-34) was used. 500 ng of each plasmid was used per transformation. After transformation, the transformed yeast cells were recovered in YPD medium for 3 hours at 30° C. and then serially diluted in PBS and plated on drop-out (omitting uracil; for the selection of PL-319, PL-320 or pUDE731) minimal agar medium (1.7 g/L bacto-yeast nitrogen base w/o amino acids and without ammonium sulfate; 1 g/L monosodium glutamate; 20 g/L glucose; 20 g/L agar) containing 200 μg/mL Geneticin (G418 sulfate) antibiotic (for the selection of PL-074 or PL-207; targeting and non-targeting plasmids) and containing different concentrations of theophylline (0, 5, 10, 20 mM).

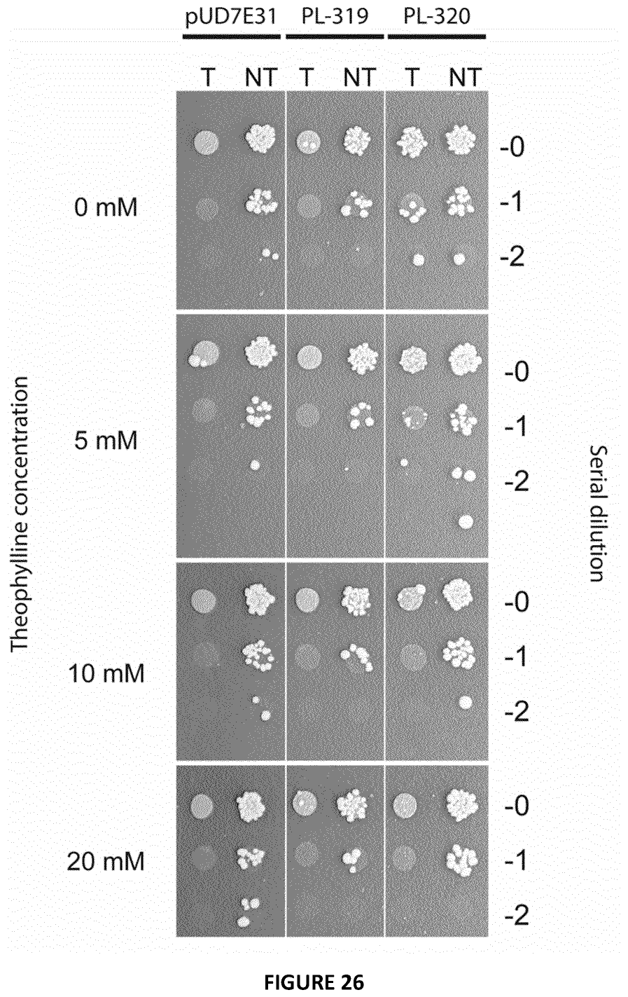

The results of this experiment are depicted in FIG. 26. S. cerevisiae cells co-transformed with PL-319, PL-320 or pUDE731 plasmids and the PL-207 non-targeting plasmid showed colony formation up to the 10-2 dilution regardless of the presence or absence of theophylline. In all these cases, colonies showed a comparable size. As expected, when the pUDE731 was co-transformed with the PL-074 targeting plasmid, almost no colonies were formed regardless of the presence or absence of theophylline. Similarly, when PL-319 was co-transformed with the PL-074 targeting plasmid, a reduced number of colonies were formed regardless of the presence or absence of theophylline. This indicates that the intron is indeed able to self-splice out of the formed FnCas12a mRNA and code for a functional FnCas12a that is able to target and cleave the target site. In contrast, when PL-320 was co-transformed with the PL-074 targeting plasmid, normal size colonies were formed only when theophylline was omitted from the agar medium. However, when theophylline (5, 10, 20 mM) was included in the agar medium, a reduced number of colonies were formed. The effect of the amount of the theophylline inducer used is also apparent as less colonies and smaller ones are visible with the increasing concentration of the theophylline inducer. This result indicated that inducible excision of the intron can be achieved in eukaryotes such as S. cerevisiae.

### Example 13: Turning any Group I Intron into a SIBR System

As noted herein above, Group I introns, like the T4 td intron, form core secondary structures consisting of multiple paired regions. In principle, to turn any Group I intron into a Self-splicing Intron Based Riboswitch (SIBR) according to the invention, a stepwise approach, for example as described herein below, can be followed, similar to the one described in this patent.

As a first step, a library of mutant 5′ and 3′ exonic sequences is developed, since the and 3′ exonic sequences of Group I introns interact with the intron sequence and affect the secondary and tertiary structure of the intron. This mutant library will serve as the basis to define the effect of the 5′ and 3′ exonic sequences on the splicing efficiency of the intron. Moreover, this library will contain introns with a range (low to high) of splicing efficiencies. It is likely that the mutant intron library will contain introns with better splicing efficiency than the wild type intron; similar to the results observed in Examples 1-3). Also, this library will allow the transfer of the intron of interest to the open reading frame of any gene of interest without disturbing the amino acid sequence of the target gene/protein, for example when applying the script of Example 4 and/or 11.

Next, to achieve inducible control over the splicing of the intron, an aptamer moiety which responds to specific small molecules (e.g. theophylline) is introduced at one or multiple pairing (P) domains of the intron. For example, as described by Thompson et al., 2002, and also shown in Examples 5 and 7, the theophylline aptamer is introduced at the P6 domain of the T4 td intron, turning it into an inducible self-splicing gene regulator. Another example is described by Kertsburg and Soukup, 2002 (Nucleic Acids Research, Volume 30, Issue 21, 1 Nov. 2002, pages 4599-4606), where they turned the Tetrahymena group I intron into an inducible self-splicing intron by replacing the P6 or P8 or both P6 and P8 domains with a theophylline aptamer. Similar approaches (to that of Thompson et al., 2002 and that of Kertsburg and Soukup, 2002) can be taken for any other Group I intron where one of their P domains is altered to contain an aptamer moiety that responds to specific small molecules and can consequently control the splicing of the intron.

After generating the mutant intron library (mutations at the 5′ and 3′ exonic sequences) and achieving inducible control over the splicing of the intron (through the introduction of an aptamer in one of the P domains of the intron), the generated intron variants can be moved to the ATG start codon, or 5′ to the start codon, of the polynucleotide portion encoding the POI, or within the polynucleotide portion encoding the POI. When transferring the intron at a location of choice, attention should be given in avoiding codon frameshifting after splicing as this will result in a non-sense protein.

### Example 14: Turning any Group II Intron into a SIBR System

Group II introns are found in higher (plants) and lower eukaryotes (fungi and yeasts) but also in bacteria. Similar to Group I introns, group II introns reside in between genes (separating them into 5′ and 3′ exons) which upon excision (formation of a lariat product instead of linear product as observed for Group I introns) allow for the formation of a functional protein. Group II introns can self-splice, although some intron-encoded proteins (IEPs) may facilitate splicing by stabilizing the intron RNA structure. The 5′ and 3′ exonic sequences of the Group II introns (called intron-binding site or IBS) interact with conserved domains of the intron (called exon-binding site or EBS) to form long-range tertiary interactions. The intron-exon interactions are necessary for splicing as they bring the intron at the active site of the exons in order to facilitate the typical transesterification reaction that mediates the excision of the intron. The necessity of intron-exon interactions for splicing, translates into a limitation in transferring any group II intron into any gene of interest (GOI), as the exon sequences need to be conserved. To overcome this, a similar approach as the one developed in this patent for Group I introns can be used.

First, a mutant library of Group II introns can be generated in which the exon sequences (IBS1 and IBS2 for 5′ exon and IBS3 for 3′ exon) are mutated. In some cases, and especially when the IBS is heavily mutated, the EBS might need to be modified as well to maintain the IBS-EBS base-pairing necessary for the formation of long-range tertiary interactions. The generated mutant library is then assessed for the efficiency of the self-splicing activity of the intron, by following a similar approach as that was employed for LacZ as described in Examples 1-3. Important to note is that self-splicing efficiency can be assessed by any other in vitro or in vivo method (other than LacZ) as long as it can distinguish the formation of spliced products from un-spliced products, or the formation and quantity of active protein from inactive proteins.

In the case where the Group II intron mutant library will be assayed through a protein (similar to that of LacZ; Examples 1-3) then, for convenience and to maintain the coding sequence of the protein, the Group II intron can be transferred directly after the ATG start codon in order to maintain the coding sequence of the protein. This approach was described in Examples 1-3 (LacZ) and Example 5 (FnCas12a). The outcome of the Group II intron mutant library assay is expected to yield a range with good and bad splicing introns which can then be used to modulate/tune the expression of the gene/RNA/protein of interest.

After establishing the requirements for splicing as defined by the IBS-EBS interactions, a script similar to Example 4 and/or 11 can be developed that allows for transferring the mutant Group II intron to virtually any gene/RNA/protein of interest.

In case inducible self-splicing is required, an aptamer moiety which responds to specific small molecules (e.g. theophylline) is introduced at one or multiple pairing (P) domains of the intron. To achieve this, the approaches developed and applied by Thompson et al. (2002) Ibid, and Kertsburg and Soukup (2002) lbid, can be used.

After generating the mutant Group II intron library (mutations at the 5′ and 3′ exonic sequences) and achieving inducible control over the splicing of the Group II intron (through the introduction of an aptamer in one of the P domains of the intron), the generated Group II intron variants can be moved at the ATG start codon, or 5′ of the ATG start of the polynucleotide portion encoding the POI, or within the polynucleotide portion encoding the POI. When transferring the intron at the location of choice, attention should be given in avoiding codon frameshifting after splicing as this will result in a non-sense protein.

### Example 15: Turning any Group III Intron to a SIBR System

In general, Group III introns are short (approx. 100 nt) U-rich introns which are predominantly found in Euglena gracilis. Group III introns are considered streamlined versions of Group II introns as they retain the 5′ splice site of group II introns but lack the catalytic domain V and the domains II-IV. To splice, a similar mechanism is used as that of Group II introns where the IBS1 pairs with EBS1 to form long-range tertiary interactions and facilitate splicing (Hong, L. and Hallick, R. B., 1994 “A group III intron is formed from domains of two individual group II introns” Genes & development, 8(13), pp. 1589-1599). In principle, Group III introns can be turned into SIBR by changing/mutating the IBS-EBS interactions as described in Example 14 and by introducing a ligand dependent aptamer to one of its domains (e.g. at the VI domain). The defined mutant libraries can then be used to modulate the splicing efficiency of the introns.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The readers attention is directed to all papers and documents which are filed concurrently with or previous to this specification in connection with this application and which are open to public inspection with this specification, and the contents of all such papers and documents are incorporated herein by reference.

Nucleotide Sequences

Sequence of WT T4 td intron (SEQ ID NO: 44):
TAATTGAGGCCTGAGTATAAGGTGACTTATACTTGTAATCTATCTAAACGGGGAACCT
CTCTAGTAGACAATCCCGTGCTAAATTGTAGGACTTGCCCTTTAATAAATACTTCTATA
TTTAAAGAGGTATTTATGAAAAGCGGAATTTATCAGATTAAAAATACTTTAAACAATAAA
GTATATGTAGGAAGTGCTAAAGATTTTGAAAAGAGATGGAAGAGGCATTTTAAAGATT
TAGAAAAAGGATGCCATTCTTCTATAAAACTTCAGAGGTCTTTTAACAAACATGGTAAT
GTGTTTGAATGTTCTATTTTGGAAGAAATTCCATATGAGAAAGATTTGATTATTGAACG
AGAAAATTTTTGGATTAAAGAGCTTAATTCTAAAATTAATGGATACAATATTGCTGATG
CAACGTTTGGTGATACATGTTCTACGCATCCATTAAAAGAAGAAATTATTAAGAAACGT
TCTGAAACTGTTAAAGCTAAGATGCTTAAACTTGGACCTGATGGTCGGAAAGCTCTTT
ACAGTAAACCCGGAAGTAAAAACGGGCGTTGGAATCCAGAAACCCATAAGTTTTGTAA
GTGCGGTGTTCGCATACAAACTTCTGCTTATACTTGTAGTAAATGCAGAAATCGTTCA
GGTGAAAATAATTCATTCTTTAATCATAAGCATTCAGACATAACTAAATCTAAAATATCA
GAAAAGATGAAAGGTAAAAAGCCTAGTAATATTAAAAAGATTTCATGTGATGGGGTTAT
TTTTGATTGTGCAGCAGATGCAGCTAGACATTTTAAAATTTCGTCTGGATTAGTTACTT
ATCGTGTAAAATCTGATAAATGGAATTGGTTCTACATAAATGCCTAACGACTATCCCTT
TGGGGAGTAGGGTCAAGTGACTCGAAACGATAGACAACTTGCTTTAACAAGTTGGAG
ATATAGTCTGCTCTGCATGGTGACATGCAGCTGGATATAATTCCGGGGTAAGATTAAC
GACCTTATCTGAACATAATG
Sequence of the WT T4 td intron with the theophylline aptamer (SEQ
ID NO: 45):
TTCTTGGGTTAATTGAGGCCTGAGTATAAGGTGACTTATACTTGTAATCTATCTAAACG
GGGAACCTCTCTAGTAGACAATCCCGTGCTAAATTGATACCAGCATCGTCTTGATGCC
CTTGGCAGCATAAATGCCTAACGACTATCCCTTTGGGGAGTAGGGTCAAGTGACTCG
AAACGATAGACAACTTGCTTTAACAAGTTGGAGATATAGTCTGCTCTGCATGGTGACA
TGCAGCTGGATATAATTCCGGGGTAAGATTAACGACCTTATCTGAACATAATGCTA
Sequence of the Tag1 T4 td intron with the theophylline aptamer
(SEQ ID NO: 49):
TCCTCAGGTTAATTGAGGCCTGAGTATAAGGTGACTTATACTTGTAATCTATCTAAACG
GGGAACCTCTCTAGTAGACAATCCCGTGCTAAATTGATACCAGCATCGTCTTGATGCC
CTTGGCAGCATAAATGCCTAACGACTATCCCTTTGGGGAGTAGGGTCAAGTGACTCG
AAACGATAGACAACTTGCTTTAACAAGTTGGAGATATAGTCTGCTCTGCATGGTGACA
TGCAGCTGGATATAATTCCGGGGTAAGATTAACGACCTTATCTGAACATAATGCTA
Sequence of the Tag2 T4 td intron with the theophylline aptamer
(SEQ ID NO: 46):
TCCTCGGGTTAATTGAGGCCTGAGTATAAGGTGACTTATACTTGTAATCTATCTAAAC
GGGGAACCTCTCTAGTAGACAATCCCGTGCTAAATTGATACCAGCATCGTCTTGATGC
CCTTGGCAGCATAAATGCCTAACGACTATCCCTTTGGGGAGTAGGGTCAAGTGACTC
GAAACGATAGACAACTTGCTTTAACAAGTTGGAGATATAGTCTGCTCTGCATGGTGAC
ATGCAGCTGGATATAATTCCGGGGTAAGATTAACGACCTTATCTGAACATAATGCTA
Sequence of the Tag3 T4 td intron with the theophylline aptamer
(SEQ ID NO: 47):
TCCTTGGGTTAATTGAGGCCTGAGTATAAGGTGACTTATACTTGTAATCTATCTAAACG
GGGAACCTCTCTAGTAGACAATCCCGTGCTAAATTGATACCAGCATCGTCTTGATGCC
CTTGGCAGCATAAATGCCTAACGACTATCCCTTTGGGGAGTAGGGTCAAGTGACTCG
AAACGATAGACAACTTGCTTTAACAAGTTGGAGATATAGTCTGCTCTGCATGGTGACA
TGCAGCTGGATATAATTCCGGGGTAAGATTAACGACCTTATCTGAACATAATGCTA
Sequence of the Tag4 T4 td intron with the theophylline aptamer
(SEQ ID NO: 48):
TCCTCTGGTTAATTGAGGCCTGAGTATAAGGTGACTTATACTTGTAATCTATCTAAACG
GGGAACCTCTCTAGTAGACAATCCCGTGCTAAATTGATACCAGCATCGTCTTGATGCC
CTTGGCAGCATAAATGCCTAACGACTATCCCTTTGGGGAGTAGGGTCAAGTGACTCG
AAACGATAGACAACTTGCTTTAACAAGTTGGAGATATAGTCTGCTCTGCATGGTGACA
TGCAGCTGGATATAATTCCGGGGTAAGATTAACGACCTTATCTGAACATAATGCTA

1. A method for controlling expression of a polypeptide of interest (POI) in a cell, comprising
A. providing a cell comprising a polynucleotide construct, the polynucleotide construct comprising:
i. a promoter functional in the cell;
ii. a polynucleotide portion encoding said P01; and
iii. a polynucleotide portion encoding at least one self-splicing intron which includes 5′ and 3′ exon nucleotide sequences, wherein the self-splicing activity of the intron is controlled by an inducer molecule;
wherein the inducer-controlled self-splicing intron is located (a) at or 5′ of the start of the polynucleotide portion encoding the POI, or (b) within the polynucleotide portion encoding the P01;
B. subjecting the cell to conditions which express polypeptides in the cell and thereby the transcription of the polynucleotide construct into RNA transcripts in the cell; and
C. subjecting the cell to conditions which cause a concentration of inducer molecule to promote the self-splicing activity of the intron in the transcripts;
thereby resulting in expression of the POI.

2. A method for controlling expression of an RNA of interest (ROI) in a cell, comprising:
A. providing a cell comprising a polynucleotide construct, the polynucleotide construct comprising:
i. a promoter functional in the cell;
ii. a polynucleotide portion encoding the ROI; and
iii. a polynucleotide portion encoding at least one self-splicing intron which includes 5′ and 3′ exon sequences, wherein the self-splicing activity of the intron is controlled by an inducer molecule;
wherein the inducer-controlled self-splicing intron is located (a) at or 5′ of the start of the polynucleotide portion encoding the ROI, or (b) within the polynucleotide portion encoding the ROI,
B. subjecting the cell to conditions which expresses the polynucleotide construct into RNA transcripts in the cell; and
C. subjecting the cell to conditions which produces a concentration of inducer molecule which promotes the self-splicing activity of the intron in the RNA transcript to produce the ROI;
thereby resulting in the expression of the ROI.

3. A method as claimed in claim 1, wherein the self-splicing intron is 3′ of and in-frame with the start codon and the expressed POI comprises an amino acid tag sequence encoded by a polynucleotide sequence which includes the 5′ and 3′ exon nucleotide sequences of the self-splicing intron rendered contiguous by self-splicing of the intron; preferably wherein the self-splicing intron is directly adjacent to the start codon and the amino acid tag sequence is an N-terminal amino acid tag in the expressed POI.

4. A method as claimed in claim 1 or claim 2, wherein the self-splicing intron is 5′ of the polynucleotide portion from which the ROI or POI is expressed and the said polynucleotide is not disrupted by the self-splicing activity of the intron; preferably wherein the self-splicing intron is 5′ of the start codon of the polynucleotide encoding the POI.

5. A method as claimed in claim 1 or claim 2, wherein the self-splicing intron is located within the polynucleotide portion encoding the ROI and preferably does not result in a tag sequence in the ROI or POI.

6. A method as claimed in any of claim 1, 3, 4 or 5, wherein the polynucleotide construct further comprises a polynucleotide sequence encoding an additional amino acid sequence.

7. A method as claimed in claim 6, wherein the additional amino acid sequence is a functional moiety, e.g. a protein purification or detection tag, a cellular localization sequence, a fluorescent moiety.

8. A method as claimed in any preceding claim, wherein there are two or more self-splicing introns located 3′ and in frame of the start codon.

9. A method as claimed in any preceding claim, wherein there is a single self-splicing intron located 5′ of the start of the polynucleotide portion encoding the ROI or POI.

10. A method as claimed in any preceding claim, wherein the inducer molecule is provided to the cell.

11. A method as claimed in any of claims 1 to 9, wherein (a) the inducer molecule is generated as a result of expression of a separate gene in the cell, wherein the separate gene is under the control of different expression regulatory elements; optionally wherein the different expression regulatory elements are responsive to a different inducer molecule and/or physical condition, e.g. temperature; or (b) wherein the inducer molecule is naturally synthesized by the cell in response to chemical and/or physical condition to which the cell is subjected to.

12. A method as claimed in any preceding claim, wherein the self-splicing intron comprises an aptamer which has binding affinity for the inducer molecule.

13. A method as claimed in any preceding claim, wherein the inducer is selected from flavin mononucleotide, thiamine pyrophosphate, s-adenosylmethionine, s-adenosylhomocysteine, adenosylcobalamin, cyclic di-GMP, adenine, guanine, glycine, lysine, theophylline, 3-methylxanthine, caffeine, 1-methylxanthine, 7-methylxanthine, 1,3-dimethyl uric acid, hypoxanthine, xanthine, theobromine tetracycline, neomycin or malachite green; preferably wherein the inducer is theophylline.

14. A method as claimed in any preceding claim, wherein the 5′ exon nucleotide sequence and/or 3′exon nucleotide sequence of the self-splicing intron are modified compared to the respective wild type exon nucleotide sequence(s) of the intron.

15. A method as claimed in any preceding claim, wherein the self-splicing intron is a group I intron.

16. A method as claimed in any of claims 1 to 14, wherein the self-splicing intron is a group II or a group III intron.

17. A method as claimed in any preceding claim, wherein the 5′ exon sequence of the self-splicing intron is NNNNNNGGT (SEQ ID NO: 3) and the 3′ exon sequence of the self-splicing intron is CTN (SEQ ID NO: 4), wherein N is A, T, C or G; optionally wherein the exon sequence is TTBYBDGGT (SEQ ID NO: 5) and the 3′ exon sequence is CTH (SEQ ID NO: 6), wherein B=G/T/C, Y=C/T, D=G/A/T and H=A/T/C optionally wherein the 5′ exon sequence is selected from TCCTCAGGT (SEQ ID NO: 7), TCCTCGGGT (SEQ ID NO: 8), TCCTTGGGT (SEQ ID NO: 9), TCCTCTGGT (SEQ ID NO: 10) or TTCTTGGGT (SEQ ID NO: 11) and the 3′ exon sequence is CTA (SEQ ID NO: 12).

18. A method as claimed in any of claims 1 or 3 to 17, wherein the POI is selected from any of:
i. a sequence specific DNA/RNA binding protein; preferably a meganuclease (MGN), zinc finger nuclease (ZFN), a TALEN, an RNA-guided nuclease or a DNA-guided nuclease;
ii. an RNA-guided nuclease; preferably a Crispr-Cas protein;
iii. a sequence-specific DNA binding protein lacking nuclease activity or a nickase; optionally fused to a heterologous functional moiety; preferably wherein the POI is a base editor or a prime editor.

19. A method as claimed in claim 18, wherein the POI is ii) or iii) and the polynucleotide further comprises a portion encoding a targeting RNA molecule, e.g. guide RNA (gRNA) which directs ii) or iii) to a target locus in a DNA sequence.

20. An isolated polynucleotide comprising:
i. a promoter functional in a cell;
ii. a polynucleotide portion encoding an RNA of interest (ROI) or a polypeptide of interest (P01); and
iii. a polynucleotide portion encoding at least one self-splicing intron which includes 5′ and 3′ exon nucleotide sequences, wherein the self-splicing activity of the intron is controlled by an inducer molecule;
wherein the inducer-controlled self-splicing intron is located (a) at or 5′ of the start of the polynucleotide portion encoding the ROI or POI, or (b) within the polynucleotide portion encoding the POI or ROI.

21. A polynucleotide as claimed in claim 20, wherein the ROI is translatable into a POI.

22. A polynucleotide as claimed in claim 20 or claim 21, wherein the self-splicing intron is 3′ of and in-frame with the start codon and a POI when expressed from the polynucleotide comprises an amino acid tag sequence encoded by a polynucleotide sequence which includes the 5′ and 3′ exon nucleotide sequences of the self-splicing intron rendered contiguous by self-splicing of the intron; preferably wherein the amino acid tag sequence is an N-terminal amino acid tag in the expressed POI.

23. A polynucleotide as claimed in claim 20 or claim 21, wherein the self-splicing intron is 5′ of the polynucleotide portion from which the ROI or POI is expressed and the said polynucleotide is not disrupted by the self-splicing activity of the intron; preferably wherein the self-splicing intron is 5′ of the start codon of the polynucleotide encoding the POI.

24. A polynucleotide as claimed in any one of claims 20 to 23, wherein the self-splicing intron is located within the polynucleotide portion encoding the ROI or POI and preferably does not result in a tag sequence in the ROI or POI.

25. A polynucleotide as claimed in any of claims 20 to 24, wherein the polynucleotide construct further comprises a polynucleotide sequence encoding an additional amino acid sequence; optionally wherein the additional amino acid sequence is a functional moiety, e.g. a protein purification or detection tag, a cellular localization sequence, a fluorescent moiety.

26. A polynucleotide as claimed in any of claims 20 to 25, wherein there is a single self-splicing intron located 5′ of the start of the polynucleotide portion encoding the ROI or POI.

27. A polynucleotide as claimed in any of claims 20 to 26, wherein the self-splicing intron comprises an aptamer which has binding affinity for the inducer molecule; optionally wherein the inducer is selected from flavin mononucleotide, thiamine pyrophosphate, s-adenosylmethionine, s-adenosylhomocysteine, adenosylcobalamin, cyclic di-GMP, adenine, guanine, glycine, lysine, theophylline, 3-methylxanthine, caffeine, 1-methylxanthine, 7-methylxanthine, 1,3-dimethyl uric acid, hypoxanthine, xanthine, theobromine tetracycline, neomycin or malachite green; preferably wherein the inducer is theophylline.

28. A polynucleotide as claimed in any of claims 20 to 27, wherein the 5′ exon nucleotide sequence and/or 3′exon nucleotide sequence of the self-splicing intron are modified compared to the respective wild type exon nucleotide sequence(s) of the intron.

29. A polynucleotide as claimed in any of claims 20 to 28, wherein the self-splicing intron is a group I intron.

30. A polynucleotide as claimed in any of claims 20 to 29, wherein the 5′ exon sequence of the self-splicing intron is NNNNNNGGT (SEQ ID NO: 3) and/or the 3′ exon sequence is CTN (SEQ ID NO: 4), wherein N is A, T, C or G; optionally wherein the 5′ exon sequence is TTBYBDGGT (SEQ ID NO: 5) and the 3′ exon sequence is CTH (SEQ ID NO: 6), wherein B=G/T/C, Y=C/T, D=G/A/T and H=A/T/C; preferably wherein the 5′ exon sequence is selected from TCCTCAGGT (SEQ ID NO: 7), TCCTCGGGT (SEQ ID NO: 8, TCCTTGGGT (SEQ ID NO: 9), TCCTCTGGT (SEQ ID NO: 10) or TTCTTGGGT (SEQ ID NO: 11) and the 3′ exon sequence is CTA (SEQ ID NO: 12).

31. A polynucleotide as claimed in any of claims 20 to 30, wherein the POI is selected from
i. a sequence specific DNA/RNA binding protein; preferably a meganuclease (MGN), zinc finger nuclease (ZFN), a TALEN, an RNA-guided nuclease or a DNA-guided nuclease;
ii. an RNA-guided nuclease; preferably a Crispr-Cas protein;
iii. a sequence-specific DNA binding protein lacking nuclease activity or a nickase; optionally fused to an heterologous functional moiety; preferably wherein the POI is a base editor or a prime editor.

32. A polynucleotide as claimed in claim 31, wherein the POI is ii) or iii) and the polynucleotide further comprises a portion encoding a targeting RNA molecule, e.g. a guide RNA (gRNA) which directs the ii) or iii) to a target locus in a DNA sequence; optionally wherein the gRNA is under the control of a self-splicing intron.

33. An expression vector comprising a polynucleotide of any of claims 20 to 32.

34. A transformed cell for inducer molecule-controlled expression of an RNA of interest (ROI) or polypeptide of interest (POI) thereby, wherein the cell comprises a polynucleotide of any of claims 20 to 32, or an expression vector of claim 33.

35. A kit for expressing an RNA of interest (ROI) or a polypeptide of interest (POI) and wherein the expression is under the control of an inducer molecule comprising:
i. a composition comprising a polynucleotide of any of claims 20 to 32, or an expression vector of claim 33, or a transformed cell of claim 34; and
ii. a composition comprising an inducer molecule which activates self-splicing activity of a self-splicing intron when expressed in a cell.

36. A system for generating an RNA of interest (ROI) or a polypeptide of interest (POI), comprising a transformed cell of claim 34.

37. A method of inducer controlled modification of a target genomic locus in a cell, comprising introducing or generating in the cell a ribonuclease complex comprising a Crispr-Cas nuclease and a gRNA molecule for the target genetic locus; wherein the Crispr-Cas nuclease and/or the gRNA is comprised as the POI and/or ROI in a polynucleotide of any of claims 20 to 32 or an expression vector of claim 33; and subjecting the cell to a condition which causes a concentration of inducer molecule to promote the self-splicing activity of the intron, thereby resulting in expression of the Crispr-Cas nuclease and/or gRNA in the cell; optionally wherein an homologous repair (HR) template encoded by the same or different polynucleotide or expression vector, and the HR template is expressed in the cell.

38. A method of inducer-controlled base editing of a target genomic locus in a cell, comprising:
A. introducing or generating in the cell a ribonuclease complex comprising a base editor and a gRNA molecule for the target genetic locus, wherein the base editor and/or gRNA is comprised as the respective ROI or POI in a polynucleotide or polynucleotides of any of claims 20 to 32 or an expression vector of claim 33; and
B. (a) providing inducer molecule to the cell, or (b) subjecting the cell to a condition which causes a concentration of inducer molecule to promote the self-splicing activity of the intron, thereby resulting in expression of the base editor and/or gRNA in the cell.

39. A method of inducer-controlled prime editing of a target genomic locus in a cell, comprising:
A. introducing or generating in the cell a ribonuclease complex comprising a prime editor and a prime editing guide RNA (pegRNA) molecule for the target genetic locus, wherein the prime editor and/or pegRNA is comprised as the respective ROI or POI in a polynucleotide or polynucleotides of any of claims 20 to 32 or an expression vector of claim 33; and
B. (a) providing inducer molecule to the cell, or (b) subjecting the cell to a condition which causes a concentration of inducer molecule to promote the self-splicing activity of the intron, thereby resulting in expression of the prime editor and/or pegRNA in the cell.

40. A method as claimed in any of claims 37 to 39, wherein the inducer molecule is provided to the cell.

41. A method as claimed in any of claims 37 to 39, wherein (a) the inducer molecule is generated as a result of expression of a separate gene in the cell, wherein the separate gene is under the control of different expression regulatory elements; optionally wherein the different expression regulatory elements are responsive to a different inducer molecule and/or physical condition, e.g. temperature; or (b) the inducer molecule is naturally synthesized by the cell in response to chemical and/or physical condition to which the cell is subjected to.

42. A method as claimed in any of claims 37 to 40, wherein a first polynucleotide comprises a self-splicing intron under the control of a first inducer molecule, and a second polynucleotide of comprises a self-splicing intron which is under the control of a second different inducer molecule.

43. A system for inducer controlled genetic modification of a cell, comprising at least a first expression vector, the first expression vector comprising a polynucleotide of any of claims 20 to 32, wherein the respective POI or ROI is selected from:
A. a Crispr-Cas nuclease, and/or
B. a gRNA, and/or
C. an HR template.

44. A system for inducer controlled genetic modification of a cell, comprising at least a first expression vector, the first expression vector comprising a polynucleotide of any of claims 20 to 32, wherein the respective POI or ROI is selected from:
A. a base editor, and/or
B. a gRNA

45. A system for inducer controlled genetic modification of a cell, comprising at least a first expression vector, the first expression vector comprising a polynucleotide of any of claims 20 to 32, wherein the respective POI or ROI is selected from:
A. a prime editor, and/or
B. a pegRNA

46. A system as claimed in any of claims 43 to 45, wherein each individual POI and/ROI is under the control of a respective self-splicing intron.

47. A system as claimed in any of claims 43 to 46, wherein a first polynucleotide comprises a self-splicing intron under the control of a first inducer molecule, and a second polynucleotide comprises a self-splicing intron which is under the control of a second different inducer molecule.


SEQUENCE LISTING (77 sequences)

SEQ ID NO: 1 - DNA, 5 bp, Unknown
  Common prokaryotic Shine-Dalgarno sequence
ggagg

SEQ ID NO: 2 - RNA, 5 nt, Unknown
  Anti- Shine-Dalgarno sequence
ccucc

SEQ ID NO: 3 - DNA, 9 bp, Artificial Sequence
  T4 td self-splicing intron 5′ exon sequence
  misc_feature (1)..(6): n is a, c, g or t
nnnnnnggt

SEQ ID NO: 4 - DNA, 3 bp, Artificial Sequence
  T4 td self-splicing intron 3′ exon sequence
  misc_feature (3)..(3): n is a, c, g or t
ctn

SEQ ID NO: 5 - DNA, 9 bp, Artificial Sequence
  T4 td self-splicing intron 5′ exon sequence
  misc_feature (3)..(6): B = G/T/C, Y = C/T, D = G/A/T
ttbybdggt

SEQ ID NO: 6 - DNA, 3 bp, Artificial Sequence
  T4 td self-splicing intron 3′ exon sequence
  misc_feature (3)..(3): H = A/T/C
cth

SEQ ID NO: 7 - DNA, 9 bp, Artificial Sequence
  T4 td self-splicing intron 5′ exon sequence
tcctcaggt

SEQ ID NO: 8 - DNA, 9 bp, Artificial Sequence
  T4 td self-splicing intron 5′ exon sequence
tcctcgggt

SEQ ID NO: 9 - DNA, 9 bp, Artificial Sequence
  T4 td self-splicing intron 5′ exon sequence
tccttgggt

SEQ ID NO: 10 - DNA, 9 bp, Artificial Sequence
  T4 td self-splicing intron 5′ exon sequence
tcctctggt

SEQ ID NO: 11 - DNA, 9 bp, Artificial Sequence
  T4 td self-splicing intron 5′ exon sequence
ttcttgggt

SEQ ID NO: 12 - DNA, 3 bp, Artificial Sequence
  T4 td self-splicing intron 3′ exon sequence
cta

SEQ ID NO: 13 - RNA, 10 nt, Artificial Sequence
  5′ exon sequence
caccuuaggu

SEQ ID NO: 14 - DNA, 4 bp, Artificial Sequence
  3′ exon sequence
cuat

SEQ ID NO: 15 - RNA, 9 nt, Artificial Sequence
  5′ exon sequence
uucuugggu

SEQ ID NO: 16 - RNA, 4 nt, Artificial Sequence
  3′ exon sequence
cuac

SEQ ID NO: 17 - DNA, 10 bp, Artificial Sequence
  5′ exon sequence
caccttaggt

SEQ ID NO: 18 - DNA, 4 bp, Artificial Sequence
  3′ exon sequence
ctat

SEQ ID NO: 19 - DNA, 10 bp, Artificial Sequence
  5′ exon sequence
tttcttgggt

SEQ ID NO: 20 - DNA, 4 bp, Artificial Sequence
  3′ exon sequence
ctac

SEQ ID NO: 21 - DNA, 7 bp, Artificial Sequence
  5′ exon sequence
caagggt

SEQ ID NO: 22 - DNA, 7 bp, Artificial Sequence
  5′ exon sequence
cttgggt

SEQ ID NO: 23 - DNA, 4 bp, Artificial Sequence
  3′ exon sequence
ctaa

SEQ ID NO: 24 - DNA, 48 bp, Artificial Sequence
  pEA001 [WT] forward
gatcttaaggatgttctcttgggttaattgaggcctgagtataaggtg

SEQ ID NO: 25 - DNA, 45 bp, Artificial Sequence
  pEA001 reverse
tgactgcagaatattaaacggtagcattatgttcagataaggtcg

SEQ ID NO: 26 - DNA, 48 bp, Artificial Sequence
  pEA001 [-7W] forward
gatcttaaggatgttcttttgggttaattgaggcctgagtataaggtg

SEQ ID NO: 27 - DNA, 48 bp, Artificial Sequence
  pEA001 [-7M] forward
gatcttaaggatgttttgttgggttaattgaggcctgagtataaggtg

SEQ ID NO: 28 - DNA, 48 bp, Artificial Sequence
  pEA001 [296P; 296W; PWW] forward
gatcttaaggatgttttcttgggttaattgaggcctgagtataaggtg

SEQ ID NO: 29 - DNA, 45 bp, Artificial Sequence
  pEA001 [296P] reverse
tgactgcagaatattaaacgggagcattatgttcagataaggtcg

SEQ ID NO: 30 - DNA, 45 bp, Artificial Sequence
  pEA001 [296W] reverse
tgactgcagaatattaaacggaagcattatgttcagataaggtcg

SEQ ID NO: 31 - DNA, 48 bp, Artificial Sequence
  pEA001 [PWM] forward
gatcttaaggatgttttctttggttaattgaggcctgagtataaggtg

SEQ ID NO: 32 - DNA, 48 bp, Artificial Sequence
  pEA001 [PMP] forward
gatcttaaggatgttttctgaggttaattgaggcctgagtataaggtg

SEQ ID NO: 33 - DNA, 48 bp, Artificial Sequence
  pEA001 [PMW] forward
gatcttaaggatgttttctggggttaattgaggcctgagtataaggtg

SEQ ID NO: 34 - DNA, 48 bp, Artificial Sequence
  pEA001 [PMM] forward
gatcttaaggatgttttctgtggttaattgaggcctgagtataaggtg

SEQ ID NO: 35 - DNA, 48 bp, Artificial Sequence
  pEA001 [MPP] forward
gatcttaaggatgttttcccaggttaattgaggcctgagtataaggtg

SEQ ID NO: 36 - DNA, 48 bp, Artificial Sequence
  pEA001 [MPW] forward
gatcttaaggatgttttcccgggttaattgaggcctgagtataaggtg

SEQ ID NO: 37 - DNA, 48 bp, Artificial Sequence
  pEA001 [MPM] forward
gatcttaaggatgttttccctggttaattgaggcctgagtataaggtg

SEQ ID NO: 38 - DNA, 48 bp, Artificial Sequence
  pEA001 [MPW] forward
gatcttaaggatgttttcctaggttaattgaggcctgagtataaggtg

SEQ ID NO: 39 - DNA, 48 bp, Artificial Sequence
  pEA001 [MWW] forward
gatcttaaggatgttttcctgggttaattgaggcctgagtataaggtg

SEQ ID NO: 40 - DNA, 48 bp, Artificial Sequence
  pEA001 [MWM] forward
gatcttaaggatgttttccttggttaattgaggcctgagtataaggtg

SEQ ID NO: 41 - DNA, 48 bp, Artificial Sequence
  pEA001 [MMP] forward
gatcttaaggatgttttccgaggttaattgaggcctgagtataaggtg

SEQ ID NO: 42 - DNA, 48 bp, Artificial Sequence
  pEA001 [MMM] forward
gatcttaaggatgttttccgtggttaattgaggcctgagtataaggtg

SEQ ID NO: 43 - DNA, 48 bp, Artificial Sequence
  pEA001 [MMW] forward
gatcttaaggatgttttccggggttaattgaggcctgagtataaggtg

SEQ ID NO: 44 - DNA, 1017 bp, Bacteriophage T4
taattgaggcctgagtataaggtgacttatacttgtaatctatctaaacggggaacctctctagtagacaatcccgtgctaaattgtaggacttgccctttaataaatacttctatatttaaagaggtatttatgaaaagcggaatttatcagattaaaaatactttaaacaataaagtatatgtaggaagtgctaaagattttgaaaagagatggaagaggcattttaaagatttagaaaaaggatgccattcttctataaaacttcagaggtcttttaacaaacatggtaatgtgtttgaatgttctattttggaagaaattccatatgagaaagatttgattattgaacgagaaaatttttggattaaagagcttaattctaaaattaatggatacaatattgctgatgcaacgtttggtgatacatgttctacgcatccattaaaagaagaaattattaagaaacgttctgaaactgttaaagctaagatgcttaaacttggacctgatggtcggaaagctctttacagtaaacccggaagtaaaaacgggcgttggaatccagaaacccataagttttgtaagtgcggtgttcgcatacaaacttctgcttatacttgtagtaaatgcagaaatcgttcaggtgaaaataattcattctttaatcataagcattcagacataactaaatctaaaatatcagaaaagatgaaaggtaaaaagcctagtaatattaaaaagatttcatgtgatggggttatttttgattgtgcagcagatgcagctagacattttaaaatttcgtctggattagttacttatcgtgtaaaatctgataaatggaattggttctacataaatgcctaacgactatccctttggggagtagggtcaagtgactcgaaacgatagacaacttgctttaacaagttggagatatagtctgctctgcatggtgacatgcagctggatataattccggggtaagattaacgaccttatctgaacataatg

SEQ ID NO: 45 - DNA, 288 bp, Artificial Sequence
  WT T4 td intron with the theophylline aptamer
ttcttgggttaattgaggcctgagtataaggtgacttatacttgtaatctatctaaacggggaacctctctagtagacaatcccgtgctaaattgataccagcatcgtcttgatgcccttggcagcataaatgcctaacgactatccctttggggagtagggtcaagtgactcgaaacgatagacaacttgctttaacaagttggagatatagtctgctctgcatggtgacatgcagctggatataattccggggtaagattaacgaccttatctgaacataatgcta

SEQ ID NO: 46 - DNA, 288 bp, Artificial Sequence
  Tag2 T4 td intron with the theophylline aptamer
tcctcaggttaattgaggcctgagtataaggtgacttatacttgtaatctatctaaacggggaacctctctagtagacaatcccgtgctaaattgataccagcatcgtcttgatgcccttggcagcataaatgcctaacgactatccctttggggagtagggtcaagtgactcgaaacgatagacaacttgctttaacaagttggagatatagtctgctctgcatggtgacatgcagctggatataattccggggtaagattaacgaccttatctgaacataatgcta

SEQ ID NO: 47 - DNA, 288 bp, Artificial Sequence
  Tag3 T4 td intron with the theophylline aptamer
tccttgggttaattgaggcctgagtataaggtgacttatacttgtaatctatctaaacggggaacctctctagtagacaatcccgtgctaaattgataccagcatcgtcttgatgcccttggcagcataaatgcctaacgactatccctttggggagtagggtcaagtgactcgaaacgatagacaacttgctttaacaagttggagatatagtctgctctgcatggtgacatgcagctggatataattccggggtaagattaacgaccttatctgaacataatgcta

SEQ ID NO: 48 - DNA, 288 bp, Artificial Sequence
  Tag4 T4 td intron with the theophylline aptamer
tcctctggttaattgaggcctgagtataaggtgacttatacttgtaatctatctaaacggggaacctctctagtagacaatcccgtgctaaattgataccagcatcgtcttgatgcccttggcagcataaatgcctaacgactatccctttggggagtagggtcaagtgactcgaaacgatagacaacttgctttaacaagttggagatatagtctgctctgcatggtgacatgcagctggatataattccggggtaagattaacgaccttatctgaacataatgcta

SEQ ID NO: 49 - DNA, 288 bp, Artificial Sequence; identical to SEQ ID NO: 46
  Tag1 T4 td intron with the theophylline aptamer

SEQ ID NO: 50 - RNA, 304 nt, Artificial Sequence
  T4 td intron
cuuggguuaauugaggccugaguauaaggugacuuauacuuguaaucuaucuaaacggggaaccucucuaguagacaaucccgugcuaaauugucaucgcuacaaggagauauaauuaaucacgacagggcccgcgaugacauaaaugccuaacgacuaucccuuuggggaguagggucaagugacucgaaacgauagacaacuugcuuuaacaaguuggagauauagucugcucugcauggugacaugcagcuggauauaauuccgggguaagauuaacgaccuuaucugaacauaaugcuac

SEQ ID NO: 51 - RNA, 51 nt, Artificial Sequence
  P6a Wild Type T4 td Intron
ugucaucgcuacaaggagauauaauuaaucacgacagggcccgcgaugaca

SEQ ID NO: 52 - RNA, 34 nt, Artificial Sequence
  P6a Theophylline Aptamer
ugauaccagcaucgucuugaugcccuuggcagca

SEQ ID NO: 53 - DNA, 38 bp, Artificial Sequence
  pSIBRFnCas12a_sprF_HR_S3 WT
  misc_feature (19)..(19): n is 999bp sprF gene
taacaattgattgccccanaacgaaaaataggatcatt

SEQ ID NO: 54 - DNA, 36 bp, Artificial Sequence
  pSIBRFnCas12a_sprF_HR_S3 mutant
taacaattgattgccccaacgaaaaataggatcatt

SEQ ID NO: 55 - DNA, 12 bp, Artificial Sequence
  Fncas12a
agttcggtttgt

SEQ ID NO: 56 - DNA, 12 bp, Artificial Sequence
  Fncas12a of SIBRCas
tcttcggtctgc

SEQ ID NO: 57 - RNA, 9 nt, Artificial Sequence
  phage T4 td intron wt
uucuugggu

SEQ ID NO: 58 - RNA, 3 nt, Artificial Sequence
  phage T4 td intron wt
cua

SEQ ID NO: 59 - RNA, 3 nt, Artificial Sequence
  phage T4 td intron mutant
  misc_feature (3)..(3): H = A/T/C
cuh

SEQ ID NO: 60 - DNA, 66 bp, Artificial Sequence
  insertion site in the WT FnCas12a gene
  Intron (33)..(34)
atggttttttatagaggagatattatcttcggtctgcattagcgaagatttattacaaaactataa

SEQ ID NO: 61 - DNA, 12 bp, Artificial Sequence
  Tag 1
atgtcctcaggt

SEQ ID NO: 62 - DNA, 12 bp, Artificial Sequence
  Tag 2
atgtcctcgggt

SEQ ID NO: 63 - DNA, 12 bp, Artificial Sequence
  Tag 3
atgtccttgggt

SEQ ID NO: 64 - DNA, 12 bp, Artificial Sequence
  Tag 4
atgtcctctggt

SEQ ID NO: 65 - DNA, 291 bp, Artificial Sequence
  Intron just after the ATG of the GOI
atgtcctcaggttaattgaggcctgagtataaggtgacttatacttgtaatctatctaaacggggaacctctctagtagacaatcccgtgctaaattgataccagcatcgtcttgatgcccttggcagcataaatgcctaacgactatccctttggggagtagggtcaagtgactcgaaacgatagacaacttgctttaacaagttggagatatagtctgctctgcatggtgacatgcagctggatataattccggggtaagattaacgaccttatctgaacataatgcta

SEQ ID NO: 66 - RNA, 291 nt, Artificial Sequence
  Translation without induction
auguccucagguuaauugaggccugaguauaaggugacuuauacuuguaaucuaucuaaacggggaaccucucuaguagacaaucccgugcuaaauugauaccagcaucgucuugaugcccuuggcagcauaaaugccuaacgacuaucccuuuggggaguagggucaagugacucgaaacgauagacaacuugcuuuaacaaguuggagauauagucugcucugcauggugacaugcagcuggauauaauuccgggguaagauuaacgaccuuaucugaacauaaugcua

SEQ ID NO: 67 - RNA, 15 nt, Artificial Sequence
  Translation with induction
auguccucaggucua

SEQ ID NO: 68 - DNA, 291 bp, Artificial Sequence
  Intron before the ATG of the GOI
tcctcaggttaattgaggcctgagtataaggtgacttatacttgtaatctatctaaacggggaacctctctagtagacaatcccgtgctaaattgataccagcatcgtcttgatgcccttggcagcataaatgcctaacgactatccctttggggagtagggtcaagtgactcgaaacgatagacaacttgctttaacaagttggagatatagtctgctctgcatggtgacatgcagctggatataattccggggtaagattaacgaccttatctgaacataatgctaatg

SEQ ID NO: 69 - RNA, 291 nt, Artificial Sequence
  Translation without induction
uccucagguuaauugaggccugaguauaaggugacuuauacuuguaaucuaucuaaacggggaaccucucuaguagacaaucccgugcuaaauugauaccagcaucgucuugaugcccuuggcagcauaaaugccuaacgacuaucccuuuggggaguagggucaagugacucgaaacgauagacaacuugcuuuaacaaguuggagauauagucugcucugcauggugacaugcagcuggauauaauuccgggguaagauuaacgaccuuaucugaacauaaugcuaaug

SEQ ID NO: 70 - RNA, 15 nt, Artificial Sequence
  Translation with induction
uccucaggucuaaug

SEQ ID NO: 71 - RNA, 9 nt, Artificial Sequence
  B = G/T/C, Y = C/T, D = G/A/T
uubybdggu

SEQ ID NO: 72 - DNA, 48 bp, Artificial Sequence
  pEA001 [PPP] forward
gatcttaaggatgttttctcaggttaattgaggcctgagtataaggtg

SEQ ID NO: 73 - DNA, 48 bp, Artificial Sequence
  pEA001 [PPW] forward
gatcttaaggatgttttctcgggttaattgaggcctgagtataaggtg

SEQ ID NO: 74 - DNA, 48 bp, Artificial Sequence
  pEA001 [PPM] forward
gatcttaaggatgttttctctggttaattgaggcctgagtataaggtg

SEQ ID NO: 75 - DNA, 48 bp, Artificial Sequence
  pEA001 [PWP] forward
gatcttaaggatgttttcttaggttaattgaggcctgagtataaggtg

SEQ ID NO: 76 - DNA, 11 bp, Artificial Sequence
  5′ exonic sequence of the intron
aaagagtcggt

SEQ ID NO: 77 - DNA, 3 bp, Artificial Sequence
  3′ exonic sequence of the intron
ctt